(19) United States
(12) Patent Application Publication
(10) Pub. No.: US 2011/0184029 A1
(43) Pub. Date: Jul. 28, 2011

(54) PYRIDONE DERIVATIVES AS NON-NUCLEOSIDE REVERSE TRANSCRIPTASE INHIBITORS

(75) Inventors: Choung U. Kim, San Carlos, CA (US); Michael L. Mitchell, Hayward, CA (US); Jong Chan Son, Daejeon (KR)

(73) Assignee: KOREA RESEARCH INSTITUTE OF CHEMICAL TECHNOLOGY, Daejeon (KR)

(21) Appl. No.: 13/054,487

(22) PCT Filed: Jul. 13, 2009
(86) PCT No.: PCT/US09/50400

§ 371 Date: Apr. 1, 2011

Related U.S. Application Data
(60) Provisional application No. 61/080,620, filed on Jul. 14, 2008.

(51) Int. Cl. A61K 31/4412; C07D 213/64; C07D 213/69; C07D 213/70; C07D 213/71; A61K 31/18

(52) U.S. Cl. 514/348; 546/298; 546/296; 514/350

(57) ABSTRACT

The present invention relates to 2-pyridone derivatives of Formula I or IV

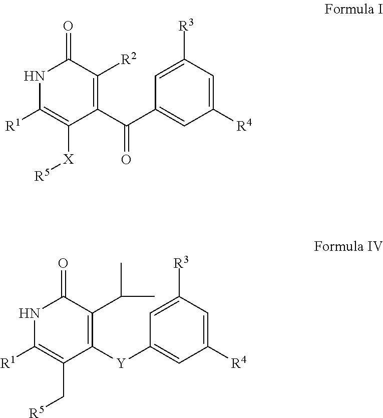

as herein described, compositions containing such compounds, synthetic processes for making such compounds, and therapeutic methods that include the administration of such compounds.

### FIELD OF THE INVENTION

The present invention relates to pyridine derivatives, as herein described, compositions containing such compounds, synthetic processes for making such compounds, and therapeutic methods that include the administration of such compounds.

### BACKGROUND OF THE INVENTION

In recent years, inhibitors of human immunodeficiency virus (HIV) reverse transcriptase (RT) have become an important class of therapeutic agents for inhibition and treatment of HIV infection in humans. Compounds that inhibit the enzymatic function of HIV RT inhibit replication of HIV in infected cells. Such compounds are useful in the prevention or treatment of HIV infection in humans, as demonstrated by known RT inhibitors such as zidovudine, didanosine, zalcitabine, stavudine, lamivudine, emtricitabine, abacavir, tenofir, nevirapine, delavirdine, and efavirenz, the main drugs thus far approved for use in the treatment of acquired immune deficiency syndrome (AIDS). Certain 2-pyridinones have also been disclosed as having anti-HIV activity and/or anti-RT activity (Tucker, et al., WO2009067166; Benjahad, et al., Bioorg. & Med. Chem. Letters (2007), 17, 712-716; Benjahad, et al., J. Med. Chem. (2005), 48, 1948-1964; Benjahad, et al., J. Med. Chem. (2004), 47, 5501-5514; Benjahad, et al., Bioorg. & Med. Chem. Letters (2003), 13, 4309-4312; Dolle, et al., J. Med. Chem. (2000), 43, 3949-3962; Pontikis, et al., J. Med. Chem. (2000), 43, 1927-1939; Dolle, et al, J. Med. Chem. (1995), 38, 4679-4686; Bisagni, et al., WO9955676; Bisagni, et al., WO9705113).

As with any antiviral therapy, use of RT inhibitors in the treatment of HIV and AIDS may lead to a virus that is less sensitive to the given drug. Resistance (reduced sensitivity) to these drugs is the result of mutations that occur in the reverse transcriptase segment of the pol gene. Several mutant strains of HIV have been characterized, and resistance to known therapeutic agents is believed to be due to mutations in the RT gene. Thus, to be effective optimally, new HIV RT inhibitors should be effective not only against wild-type strains of HIV, but should also be effective against the newly emerging mutant strains that are resistant to the commercially available RT inhibitors.

Accordingly, there continues to be a need for new HIV RT inhibitors, for example those targeting the HIV RT in both wild type and mutant strains of HIV.

### SUMMARY OF THE INVENTION

In one embodiment, the present invention is a compound of Formula I:

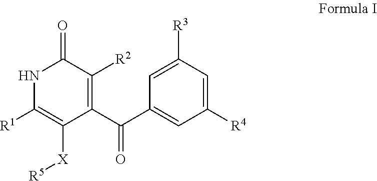

or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R² is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
X is alkylene, substituted alkylene, alkenylene, substituted alkenylene, alkynylene, or substituted alkynylene;
R⁵ is cycloalkyl, substituted cycloalkyl, aryl, substituted aryl, heterocyclyl, substituted heterocyclyl, heteroaryl, or substituted heteroaryl;
or R¹ and R⁵ and X can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

Another aspect of the present invention is a pharmaceutical composition comprising a compound of the present invention and one or more pharmaceutically acceptable carrier. In one embodiment, the composition includes one or more additional therapeutic agent. In a further embodiment, the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

Another aspect of the present invention is a method for inhibiting HIV reverse transcriptase comprising the administration of a compound of the present invention. A further aspect is a method for the treatment or prevention of HIV infection comprising the administration of a compound of the present invention. A further aspect of the present invention is a method for treating or preventing AIDS or AIDS-Related Complex comprising the administration of a compound of the present invention. Another aspect of the present invention is a method for inhibiting replication of a retrovirus comprising the administration of a compound of the present invention. In one embodiment, such methods further include the administration of one or more additional therapeutic agent. In a further embodiment, the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

Another aspect of the present invention is a compound substantially as herein described by one or more Examples.

Another aspect of the present invention is a compound of the present invention for use as a therapeutic substance.

Another aspect of the present invention is use of a compound of the present invention in the manufacture of a medicament for inhibiting HIV reverse transcriptase. A further aspect is use of a compound of the present invention in the manufacture of a medicament for treatment or prevention of HIV infection. A further aspect of the present invention is use of a compound of the present invention in the manufacture of a medicament for treating or preventing AIDS or AIDS-Related Complex. A further aspect of the present invention is use of a compound of the present invention in the manufacture of a medicament for inhibiting replication of a retrovirus. In one embodiment, such uses further include the use of one or more additional therapeutic agent. In a further embodiment, the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

Another aspect of the present invention is a compound of the present invention for use in inhibiting HIV reverse transcriptase. A further aspect is a compound of the present invention for use in the treatment or prevention of HIV infection. A further aspect is a compound of the present invention for use in treating or preventing AIDS or AIDS-Related Complex. A further aspect is a compound of the present invention for use in inhibiting replication of a retrovirus. In one embodiment, the compound further includes one or more additional therapeutic agent. In a further embodiment, the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

The scope of the present invention includes all combinations of aspects and embodiments.

### DETAILED DESCRIPTION

In one embodiment of Formula I, R¹ is alkyl, alkynyl, haloalkyl, (Q)ₘ-hydroxy, or (Q)ₘ-cyano. In another aspect of this embodiment, R¹ is alkyl. In another aspect of this embodiment, R¹ is methyl.

In one embodiment of Formula I, R² is alkyl. In a further embodiment, R² is isopropyl.

In one embodiment of Formula I, R³ is alkyl, haloalkyl, halogen, cyano, nitro, amino, alkylamino, or dialkylamino. In another aspect of this embodiment, R³ is methyl, chloro, or cyano.

In one embodiment of Formula I, R⁴ is alkyl, substituted alkynyl, haloalkyl, halogen, cyano, —C(O)OR¹⁰, or —C(O)N(R¹⁰)₂. In another aspect of this embodiment, R⁴ is methyl, chloro, or cyano.

In another embodiment of Formula I, each R³ and R⁴ is independently selected from methyl, chloro or cyano. In another aspect of this embodiment, R² is isopropyl. In another aspect of this embodiment, R¹ is methyl and R² is isopropyl.

In one embodiment of Formula I, X is alkylene. In a further embodiment, X is methylene.

In one embodiment of Formula I, R⁵ is cycloalkyl or substituted cycloalkyl. In a further embodiment, R⁵ is cycloalkyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl or substituted cyclopropyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene.

In another embodiment of Formula I, X is methylene and R⁵ is cyclopropyl or substituted cyclopropyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene. In another aspect of this embodiment, R² is isopropyl. In another aspect of this embodiment, R¹ is methyl and R² is isopropyl.

In another embodiment of Formula I, R⁵ is

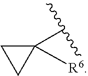

In another aspect of this embodiment, R⁶ is selected from (Q)ₘ-hydroxyl, —C(O)R¹⁰, (Q)ₘ-OC(O)R¹⁰ or (Q)ₘ-cyano. In another aspect of this embodiment R⁶ is (Q)ₘ-hydroxyl, (Q)ₘ-OC(O)R¹⁰ or (Q)ₘ-cyano wherein m is 1 or 0. In another aspect of this embodiment, R⁶ is selected from —OH, —CH₂OH, —C(O)H, —CN, or —CH₂CN. In another aspect of this embodiment, R¹ is methyl. In another aspect of this embodiment, each R³ and R⁴ is independently selected from methyl, chloro or cyano. In another aspect of this embodiment, each R³ and R⁴ is independently selected from methyl, chloro or cyano and R¹ is methyl.

In one embodiment of Formula I, each Q is independently alkylene wherein m is 0 or 1.

In another embodiment, the present invention is a compound of Formula II:

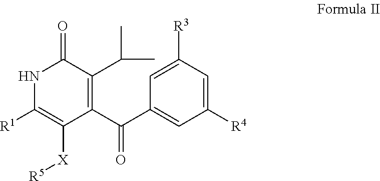

or a pharmaceutically acceptable salt or prodrug thereof, wherein R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
X is alkylene, substituted alkylene, alkenylene, substituted alkenylene, alkynylene, or substituted alkynylene;
R⁵ is cycloalkyl, substituted cycloalkyl, aryl, substituted aryl, heterocyclyl, substituted heterocyclyl, heteroaryl, or substituted heteroaryl;
or R¹ and R⁵ and X can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

In one embodiment of Formula II, R¹ is alkyl, alkynyl, haloalkyl, (Q)ₘ-hydroxy, or (Q)ₘ-cyano. In another aspect of this embodiment, R¹ is alkyl. In another aspect of this embodiment, R¹ is methyl.

In one embodiment of Formula II, R³ is alkyl, haloalkyl, halogen, cyano, nitro, amino, alkylamino, or dialkylamino. In another aspect of this embodiment, R³ is methyl, chloro, or cyano.

In one embodiment of Formula II, R⁴ is alkyl, substituted alkynyl, haloalkyl, halogen, cyano, —C(O)OR¹⁰, or —C(O)N(R¹⁰)₂. In another aspect of this embodiment, R⁴ is methyl, chloro, or cyano.

In another embodiment of Formula II, each R³ and R⁴ is independently selected from methyl, chloro or cyano. In another aspect of this embodiment, R¹ is methyl.

In one embodiment of Formula II, X is alkylene. In a further embodiment, X is methylene.

In one embodiment of Formula II, R⁵ is cycloalkyl or substituted cycloalkyl. In a further embodiment, R⁵ is cycloalkyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl or substituted cyclopropyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene.

In another embodiment of Formula II, X is methylene and R⁵ is cyclopropyl or substituted cyclopropyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene. In another aspect of this embodiment, R¹ is methyl. In another aspect of this embodiment, each R³ and R⁴ is independently selected from methyl, chloro or cyano.

In another embodiment of Formula II, R⁵ is

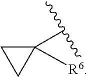

In another aspect of this embodiment, R⁶ is selected from (Q)ₘ-hydroxyl, —C(O)R¹⁰, (Q)ₘ-OC(O)R¹⁰ or (Q)ₘ-cyano. In another aspect of this embodiment R⁶ is (Q)ₘ-hydroxyl, (Q)ₘ-OC(O)R¹⁰ or (Q)ₘ-cyano wherein m is 1 or 0. In another aspect of this embodiment, R⁶ is selected from —OH, —CH₂OH, —C(O)H, —CN, or —CH₂CN. In another aspect of this embodiment, R¹ is methyl. In another aspect of this embodiment, each R³ and R⁴ is independently selected from methyl, chloro or cyano. In another aspect of this embodiment, each R³ and R⁴ is independently selected from methyl, chloro or cyano and R¹ is methyl.

In one embodiment of Formula II, each Q is independently alkylene wherein m is 0 or 1.

In another embodiment, the present invention is a compound of Formula III:

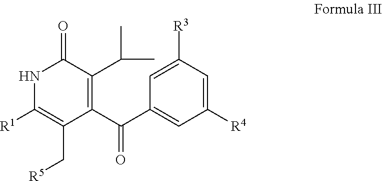

or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁵ is cycloalkyl, substituted cycloalkyl, aryl, substituted aryl, heterocyclyl, substituted heterocyclyl, heteroaryl, or substituted heteroaryl;
or R¹ and R⁵ can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

In one embodiment of Formula III, R¹ is alkyl, alkynyl, haloalkyl, (Q)ₘ-hydroxy, or (Q)ₘ-cyano. In another aspect of this embodiment, R¹ is alkyl. In another aspect of this embodiment, R¹ is methyl.

In one embodiment of Formula III, R³ is alkyl, haloalkyl, halogen, cyano, nitro, amino, alkylamino, or dialkylamino. In another aspect of this embodiment, R³ is methyl, chloro, or cyano.

In one embodiment of Formula III, R⁴ is alkyl, substituted alkynyl, haloalkyl, halogen, cyano, —C(O)OR¹⁰, or —C(O)N(R¹⁰)₂. In another aspect of this embodiment, R⁴ is methyl, chloro, or cyano.

In another embodiment of Formula III, each R³ and R⁴ is independently selected from methyl, chloro or cyano. In another aspect of this embodiment, R¹ is methyl.

In one embodiment Formula III, R⁵ is cycloalkyl or substituted cycloalkyl. In another aspect of this embodiment, R⁵ is cyclopropyl or substituted cyclopropyl. In another aspect of this embodiment, R⁵ is cyclopropyl.

In one embodiment of Formula III, R⁵ is cycloalkyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1.

In another embodiment of Formula III, R⁵ is cyclopropyl or substituted cyclopropyl and R¹ is methyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene. In another aspect of this embodiment, each R³ and R⁴ is independently selected from methyl, chloro or cyano.

In another embodiment of Formula III, R⁵ is

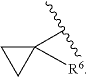

In another aspect of this embodiment, R⁶ is selected from (Q)ₘ-hydroxyl, —C(O)R¹⁰, (Q)ₘ-OC(O)R¹⁰ or (Q)ₘ-cyano. In another aspect of this embodiment R⁶ is (Q)ₘ-hydroxyl, (Q)ₘ-OC(O)R¹⁰ or (Q)ₘ-cyano wherein m is 1 or 0. In another aspect of this embodiment, R⁶ is selected from —OH, —CH₂OH, —C(O)H, —CN, or —CH₂CN. In another aspect of this embodiment, R¹ is methyl. In another aspect of this embodiment, each R³ and R⁴ is independently selected from methyl, chloro or cyano. In another aspect of this embodiment, each R³ and R⁴ is independently selected from methyl, chloro or cyano and R¹ is methyl.

In one embodiment of Formula III, each Q is independently alkylene wherein m is 0 or 1.

In another embodiment, the present invention is a compound of Formula IV:

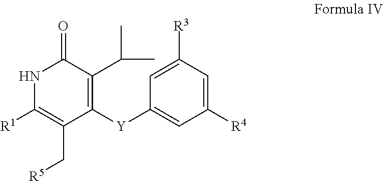

or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;

### Y is —O(O)—, —S(O)_{q}—, —O—, —N(R¹⁰)—, —C(R¹⁰)₂—, or —CF₂—;

R⁵ is cycloalkyl, substituted cycloalkyl, aryl, substituted aryl, heterocyclyl, substituted heterocyclyl, heteroaryl, or substituted heteroaryl;
or R¹ and R⁵ can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

In one embodiment of Formula IV, Y is —C(O)—, —S(O)_{q}—, —O—, —N(R¹⁰)—, —C(R¹⁰)₂—, or —CF₂—. In one aspect of this embodiment, Y is —C(O)—. In one aspect of this embodiment, Y is —S(O)_{q}—. In one aspect of this embodiment, Y is —O—. In one aspect of this embodiment, Y is —N(R¹⁰)—. In one aspect of this embodiment, Y is —O(R¹⁰)₂—. In one aspect of this embodiment, Y is —CF₂—.

In one embodiment of Formula IV, Y is —C(O)— or —S(O)_{q}—.

In one embodiment of Formula IV, R¹ is alkyl, alkynyl, haloalkyl, (Q)ₘ-hydroxy, or (Q)ₘ-cyano. In another aspect of this embodiment, R¹ is alkyl. In another aspect of this embodiment, R¹ is methyl.

In one embodiment of Formula IV, R³ is alkyl, haloalkyl, halogen, cyano, nitro, amino, alkylamino, or dialkylamino. In another aspect of this embodiment, R³ is methyl, chloro, or cyano.

In one embodiment of Formula IV, R⁴ is alkyl, substituted alkynyl, haloalkyl, halogen, cyano, —C(O)OR¹⁰, or —C(O)N(R¹⁰)₂. In another aspect of this embodiment, R⁴ is methyl, chloro, or cyano.

In another embodiment of Formula IV, each R³ and R⁴ is independently selected from methyl, chloro or cyano. In another aspect of this embodiment, R¹ is methyl.

In one embodiment Formula IV, R⁵ is cycloalkyl or substituted cycloalkyl. In another aspect of this embodiment, R⁵ is cyclopropyl or substituted cyclopropyl.

In one embodiment of Formula IV, R⁵ is cycloalkyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0 and Q is methylene.

In another embodiment of Formula IV, R⁵ is cyclopropyl or substituted cyclopropyl and R¹ is methyl. In another aspect of this embodiment, R⁵ is cyclopropyl substituted with one or more (Q)ₘ-hydroxyl, —C(O)R¹⁰, (Q)ₘ-OC(O)R¹⁰ or (Q)ₘ-cyano. In another aspect of this embodiment, R⁵ is cyclopropyl substituted with one or more (Q)ₘ-hydroxyl, (Q)ₘ-OC(O)R¹⁰ or (Q)ₘ-cyano wherein m is 1 or 0. In another aspect of this embodiment, R⁵ is cyclopropyl substituted with one or more (Q)ₘ-hydroxyl, (Q)ₘ-OC(O) R¹⁰ or (Q)ₘ-cyano wherein m is 1 or 0 and Q is methylene. In another aspect of this embodiment, each R³ and R⁴ is independently selected from methyl, chloro or cyano.

In one embodiment of Formula IV, each Q is independently alkylene wherein m is 0 or 1.

In another embodiment, the present invention includes a compound of Formula V:

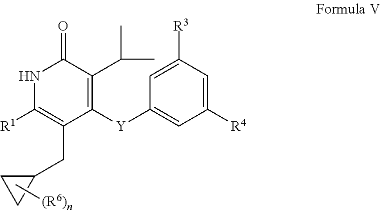

or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;

### Y is —O(O)—, —S(O)_{q}—, —O—, —N(R¹⁰)—, —O(R¹⁰)₂—, or —CF₂—;

wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (O)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO_{2R}¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O) R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
n is 0, 1, 2, or 3;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

In one embodiment of Formula V, Y is —C(O)—, —S(O)_{q}—, —O—, —N(R¹⁰)—, —O(R¹⁰)₂—, or —CF₂—. In one aspect of this embodiment, Y is —C(O)—. In one aspect of this embodiment, Y is —S(O)_{q}—. In one aspect of this embodiment, Y is —O—. In one aspect of this embodiment, Y is —N(R¹⁰)—. In one aspect of this embodiment, Y is —O(R¹⁰)₂—. In one aspect of this embodiment, Y is —CF₂—.

In one embodiment of Formula V, R¹ is alkyl, alkynyl, haloalkyl, (Q)ₘ-hydroxy, or (Q)ₘ-cyano. In another aspect of this embodiment, R¹ is —CH₃, —CF₃, —CH₂CF₃, —CH₂OH, —CH₂CN, or —CH₂C≡CH. In another aspect of this embodiment, R¹ is alkyl. In another aspect of this embodiment, R¹ is methyl.

In one embodiment of Formula V, R³ is alkyl, haloalkyl, halogen, cyano, nitro, amino, alkylamino, or dialkylamino. In another aspect of this embodiment, R³ is —CH₃, —CN, —Cl, —NO₂, or —NH₃. In another aspect of this embodiment, R³ is methyl, chloro, or cyano.

In one embodiment of Formula V, R⁴ is alkyl, substituted alkynyl, haloalkyl, halogen, cyano, —C(O)OR¹⁰, or —C(O)N(R¹⁰)₂. In another aspect of this embodiment, R⁴ is —CH₃, —CN, —Br, —I, —C≡C—CH₂OH, —C(O)OCH₃, or —C(O)NH₂. In another aspect of this embodiment, R⁴ is methyl, chloro, or cyano.

In another embodiment of Formula V, each R³ and R⁴ is independently selected from methyl, chloro or cyano. In another aspect of this embodiment, R¹ is methyl.

In one embodiment of Formula V, n is 1 such that the depicted cyclopropyl group is substituted with an R⁶ selected from (Q)ₘ-hydroxyl, —C(O)R¹⁰, (Q)ₘ-OC(O)R¹⁰ or (Q)ₘ-cyano. In another aspect of this embodiment R⁶ is (Q)ₘ-hydroxyl, (Q)ₘ-OC(O)R¹⁰ or (Q)ₘ-cyano wherein m is 1 or 0. In another aspect of this embodiment, R⁶ is selected from —OH, —CH₂OH, —C(O)H, —CN, or —CH₂ON.

In another embodiment of Formula V, the depicted cyclopropyl group is

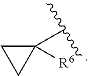

In another aspect of this embodiment, R⁶ selected from (Q)ₘ-hydroxyl, —C(O)R¹⁰, (Q)ₘ-OC(O)R¹⁰ or (Q)ₘ-cyano. In another aspect of this embodiment R⁶ is (Q)ₘ-hydroxyl, (Q)ₘ-OC(O)R¹⁰ or (Q)ₘ-cyano wherein m is 1 or 0. In another aspect of this embodiment, R⁶ is selected from —OH, —CH₂OH, —C(O)H, —CN, or —CH₂CN. In another aspect of this embodiment, R¹ is methyl. In another aspect of this embodiment, each R³ and R⁴ is independently selected from methyl, chloro or cyano. In another aspect of this embodiment, each R³ and R⁴ is independently selected from methyl, chloro or cyano and R¹ is methyl.

Variables groups in the compounds of Formulae I-V, for example R¹, R², R³, R⁴, X, R⁵, R⁶Q, and R¹⁰, may be defined interms of alkyl, alkenyl, alkynyl, alkoxy, cycloalkyl, alkylene, alkenylene, alkynylene and substituted variations thereof as described above. The preferred ranges of alkyl, alkenyl, alkynyl, alkoxy, cycloalkyl, alkylene, alkenylene, alkynylene and substituted variations thereof are detailed below in the definitions.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, may be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl. In one embodiment, the compounds preferably include C₁₋₁₂ alkyl, more preferably C₁₋₈ alkyl, and more preferably C₁₋₄ alkyl.

As used herein the term “alkenyl” refers to a straight or branched chain aliphatic hydrocarbon containing one or more carbon-to-carbon double bonds, which may be optionally substituted, with multiple degrees of substitution being allowed. Examples of “alkenyl” as used herein include, but are not limited to, vinyl, and allyl. In one embodiment, the compounds preferably include C₂₋₁₂ alkenyl, more preferably C₂₋₈ alkenyl, and more preferably C₂₋₄ alkenyl. Examples of suitable alkenyl groups include, but are not limited to, ethylene or vinyl (—CH═CH₂), allyl (—CH₂CH═CH₂), cyclopentenyl (—O₅H₇), and 5-hexenyl (—CH₂CH₂CH₂CH₂CH═CH₂).

As used herein the term “alkynyl” refers to a straight or branched chain aliphatic hydrocarbon containing one or more carbon-to-carbon triple bonds, which may be optionally substituted, with multiple degrees of substitution being allowed. An example of “alkynyl” as used herein includes, but is not limited to, ethynyl. In one embodiment, the compounds preferably include C₂₋₁₂ alkynyl, more preferably C₂₋₈ alkynyl, and more preferably C₂₋₄ alkynyl. Other examples of suitable alkynyl groups include, but are not limited to, ethynyl (—C≡CH), propargyl (—CH₂C≡CH), and the like.

As used herein the term “alkylene” refers to a straight or branched divalent chain hydrocarbon, which may be optionally substituted, with multiple degrees of substitution being allowed. Examples of “alkylene” as used herein include, but are not limited to, methylene, ethylene, and propylene. In one embodiment, the compounds preferably include C₁₋₁₂ alkylene, more preferably C₁₋₈ alkylene, more preferably C₁₋₄ alkylene, and more preferably —CH₂—. Other typical alkylene radicals include, but are not limited to, methylene (—CH₂—), 1,1-ethylene (—CH(CH₃)—), 1,2-ethylene (—CH₂CH₂—), 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), and the like.

As used herein the term “alkenylene” refers to a straight or branched divalent chain aliphatic hydrocarbon containing one or more carbon-to-carbon double bonds, which may be optionally substituted, with multiple degrees of substitution being allowed. Examples of “alkenylene” as used herein include, but are not limited to, ethene-1,2-diyl, propene-1,3-diyl, methylene-l, 1-diyl, and the like. In one embodiment, the compounds preferably include C₂₋₁₂ alkenylene, more preferably C₂₋₈ alkenylene, and more preferably C₂₋₄ alkenylene.

As used herein the term “alkynylene” refers to a straight or branched divalent chain aliphatic hydrocarbon containing one or more carbon-to-carbon triple bonds, which may be optionally substituted, with multiple degrees of substitution being allowed. An example of “alkynylene” as used herein includes, but is not limited to, ethyne-1,2-diyl and propyne-1,3-diyl. In one embodiment, the compounds preferably include C₂₋₁₂ alkynylene, more preferably C₂₋₈ alkynylene, and more preferably C₂₋₄ alkynylene. Other typical alkynylene radicals include, but are not limited to, ethyn-1,2-diyl (—C≡C—), 2-propyn-1,3-diyl I(—CH₂C≡C—), and 4-pentyn-1,5-diyl (—CH₂CH₂CH₂C≡C—).

As used herein, the term “cycloalkyl” refers to a fully saturated or partially unsaturated (e.g., cycloakenyl, cycloalkadienyl, etc.) monocyclic, bicyclic, polycyclic or bridged hydrocarbon ring, with multiple degrees of substitution being allowed for each. Preferably the cycloalkyl groups will have 3 to 8 carbon atoms as a monocycle, 7 to 12 carbon atoms as a bicycle, and up to about 20 carbon atoms as a polycycle. Bicyclic cycloalkyls have 7 to 12 ring atoms, e.g., arranged as a bicyclo [4,5], [5,5], [5,6] or [6,6] system, or 9 or 10 ring atoms arranged as a bicyclo [5,6] or [6,6] system, or spiro-fused rings. Non-limiting examples of bridged hydrocarbon rings include bicyclo[1.1.1]pentyl, bicyclo[3.2.0]heptyl, bicyclo[3.1.0]hexyl, bicyclo[2.2.1]heptyl and bicyclo[3,2,1]octyl. Non-limiting examples of monocyclic cycloalkyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, and 1-cyclohex-3-enyl. In one embodiment, the compounds preferably include C₃₋₁₂ cycloalkyl, and more preferably C₃₋₈ cycloalkyl.

As used herein, the term “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene. The term also includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566.

By way of example and not limitation, carbon bonded heterocycles are bonded at position 2, 3, 4, 5, or 6 of a piperidine; position 3, 4, 5, or 6 of a tetrahydropyridazine; position 2, 3, 5, or 6 of a piperazine; position 2, 3, 4, or 5 of a tetrahydrofuran, tetrahydrothiophene, or tetrahydropyrrole; position 2 or 3 of an aziridine; or position 2, 3, or 4 of an azetidine.

By way of example and not limitation, nitrogen bonded heterocycles are bonded at position 1 of an aziridine, azetidine, pyrrolidine, 2-pyrroline, 3-pyrroline, imidazolidine, 2-imidazoline, 3-imidazoline, pyrazoline, 2-pyrazoline, 3-pyrazoline, piperidine, piperazine, indoline, or position 4 of a morpholine.

“Aryl” means an aromatic hydrocarbon radical derived by the removal of one hydrogen atom from a single carbon atom of a parent aromatic ring system wherein the aryl may be optionally substituted, with multiple degrees of substitution being allowed. For example, an aryl group can have 6 to 20 carbon atoms, 6 to 14 carbon atoms, or 6 to 12 carbon atoms. Typical aryl groups include, but are not limited to, radicals derived from benzene (e.g., phenyl), substituted benzene, naphthalene, anthracene, phenanthrene, biphenyl, and the like. Preferable aryl rings have five- to ten-members and are more preferably optionally substituted phenyl.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic five to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃. In one embodiment, the compounds preferably include C₁₋₁₂ haloalkyl, more preferably C₁₋₈ haloalkyl, more preferably C₁₋₄ haloalkyl, and more preferably —CF₃.

As used herein the term “alkoxy” refers to a group —OR^{a}, wherein R^{a} is alkyl or cycloalkyl as defined above.

As used herein the term “nitro” refers to a group —NO₂.

As used herein the term “cyano” refers to a group —CN.

As used herein “amino” refers to a group —NR^{a}R^{b}, wherein each of R^{a} and R^{b} individually and independently is hydrogen, alkyl, alkenyl, alkynyl, cycloalkyl, aryl, heterocylcyl, or heteroaryl as defined herein. As used herein, when either R^{a} or R^{b} is other than hydrogen, such a group may be referred to as a “substituted amino” or, for example if R^{a} is H and R^{b} is alkyl, as an “alkylamino” or, in the case with both R^{a} and R^{b} are alkyl, as a “dialkylamino.”

As used herein, the term “hydroxyl” or “hydroxy” refers to a group —OH.

When trade names are used herein, such use incorporates the tradename product as well as the active pharmaceutical ingredient(s) within such product.

The term “optionally substituted” in reference to a particular moiety of the compound of Formula I-V (e.g., an optionally substituted aryl group) refers to a moiety wherein all substiutents are hydrogen or wherein one or more of the hydrogens of the moiety may be replaced by substituents such as those listed under the definition of “substituted”.

Selected substituents comprising the compounds of Formula I-V may be present to a recursive degree. In this context, “recursive substituent” means that a substituent may recite another instance of itself. Because of the recursive nature of such substituents, theoretically, a large number of compounds may be present in any given embodiment. For example, Q comprises a substituted alkylene group. A substituent of a substituted alkylene comprises an R⁶ group and R⁶ can comprise a Q group. One of ordinary skill in the art of medicinal chemistry understands that the total number of such substituents is reasonably limited by the desired properties of the compound intended. Such properties include, by way of example and not limitation, physical properties such as molecular weight, solubility or log P, application properties such as activity against the intended target, and practical properties such as ease of synthesis. By way of example and not limitation, Q and R⁶ are recursive variables in certain embodiments. Typically, each recursive substituent can independently occur 20, 19, 18, 17, 16, 15, 14, 13, 12, 11, 10, 9, 8, 7, 6, 5, 4, 3, 2, 1, or 0, times in a given embodiment. Preferably, recursive variables will independently occur 3 times or less.

The term “substituted” in reference to alkyl, alkynyl alkylene, aryl, arylalkyl, alkoxy, heterocyclyl, heteroaryl, carbocycle, cycloalkyl, etc., for example, “substituted alkyl”, “substituted alkynyl”, “substituted alkylene”, “substituted aryl”, “substituted arylalkyl”, “substituted heterocyclyl”, “substituted carbocyclyl” and “substituted cycloalkyl” means alkyl, alkynyl, alkylene, aryl, arylalkyl, heterocyclyl, carbocyclyl and cycloalkyl, respectively, in which one or more hydrogen atoms are each independently replaced with a non-hydrogen substituent. Unless otherwise defined, typical substituents include, but are not limited to, —X¹, —R^{c}, —O⁻, ═O, —OR^{c}, —SR^{c}, —S⁻, —NR^{c}₂, —N⁺R^{c}₃, ═NR^{c}, —C(X¹)₃, —CN, —OCN, —SCN, —N═C═O, —NCS, —NO, —NO₂, ═N₂, —N₃, —NHC(═O)R^{c}, —OC(═O)R^{c}, —NHC(═O)NR^{c}₂, —S(═O)₂⁻, —S(═O)₂OH, —S(═O)₂R^{c}, —OS (═O)₂OR^{c}, —S(═O)₂NR^{c}₂, —S(═O)R^{c}, —OP(═O)(OR^{c})₂, —P(═O)(OR^{c})₂, —P(═O)(O⁻)₂, —P(═O)(OH)₂, —P(O)(OR^{c})(O⁻), —C(═O)R^{c}, —C(═O)X, —C(S)R^{c}, —C(O)OR^{c}, —C(O)O⁻, —C(S)OR^{c}, —C(O)SR^{c}, —C(S)SR^{c}, —C(O)NR^{c}₂, —C(S)NR^{c}₂, —C(═NR^{c})NR^{c}₂, where each X¹ is independently a halogen: F, Cl, Br, or I; and each R^{c} is independently H, alkyl, aryl, arylalkyl, a heterocycle, or a protecting group or prodrug moiety; as defined herein. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

As used herein, the term “prodrug” refers to a derivative of a compound of the present invention such that when administered to a biological system generates a compound of the present invention as a result of a spontaneous chemical reaction(s), enzyme catalyzed chemical reaction(s), photolysis, and/or metabolic chemical reaction(s). A prodrug, thus, is a covalently modified analog or latent form of a therapeutically active compound of the formulae herein described.

Unless otherwise specified, the carbon atoms of this invention are intended to have a valence of four. In some chemical structure representations where carbon atoms do not have a sufficient number of variables attached to produce a valence of four, the remaining carbon substitutents needed to provide a valence of four should be assumed to be hydrogen.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

A compound of Formula I-V and its pharmaceutically acceptable salts may exist as different polymorphs or pseudopolymorphs. As used herein, crystalline polymorphism means the ability of a crystalline compound to exist in different crystal structures. The crystalline polymorphism may result from differences in crystal packing (packing polymorphism) or differences in packing between different conformers of the same molecule (conformational polymorphism). As used herein, crystalline pseudopolymorphism means the ability of a hydrate or solvate of a compound to exist in different crystal structures. The pseudopolymorphs of the instant invention may exist due to differences in crystal packing (packing pseudopolymorphism) or due to differences in packing between different conformers of the same molecule (conformational pseudopolymorphism). The instant invention comprises all polymorphs and pseudopolymorphs of the compounds of Formula I-V and their pharmaceutically acceptable salts.

A compound of Formula I-V and its pharmaceutically acceptable salts may also exist as an amorphous solid. As used herein, an amorphous solid is a solid in which there is no long-range order of the positions of the atoms in the solid. This definition applies as well when the crystal size is two nanometers or less. Additives, including solvents, may be used to create the amorphous forms of the instant invention. The instant invention comprises all amorphous forms of the compounds of Formula I-V and their pharmaceutically acceptable salts.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994), incorporated by reference with regard to stereochemistry.

As described, the compounds of the present invention include pyridone derivatives. Thus, the compounds of the present invention may exist in tautomeric forms. The preferred tautomeric form is as depicted in the formulae illustrated herein. Nevertheless, the scope of the present invention includes both mixtures of tautomers, as well as enriched mixtures, or an isolated tautomer. Thus, the scope of the present invention includes each alternative tautomeric form of each of the formulae herein described. For example, the scope of the present invention includes

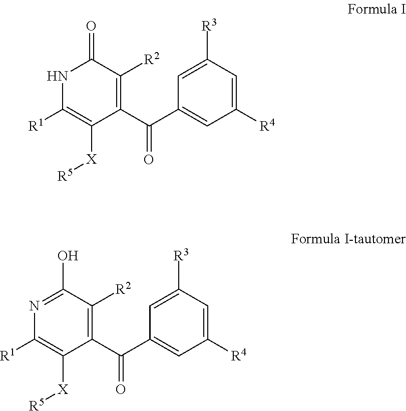

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention. However, salts of acids or bases which are not physiologically acceptable may also find use, for example, in the preparation or purification of a physiologically acceptable compound. All salts, whether or not derived form a physiologically acceptable acid or base, are within the scope of the present invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

Finally, it is to be understood that the compositions herein comprise compounds of the invention in their un-ionized, as well as zwitterionic form, and combinations with stoichiometric amounts of water as in hydrates.

### Pharmaceutical Compositions/Formulations

While it is possible for the active ingredients of the invention to be administered alone it may be preferable to present them as pharmaceutical formulations. The pharmaceutical compositions described herein include one or more compounds and/or pharmaceutically acceptable salts thereof. The pharmaceutical compositions of the instant invention comprise at least one active ingredient, as above defined, together with one or more pharmaceutically acceptable carriers and optionally other therapeutic ingredients. The carrier(s) must be “acceptable” in the sense of being compatible with the other ingredients of the formulation and physiologically innocuous to the recipient thereof. The resulting pharmaceutical compositions can be used to prevent a condition or disorder in a subject susceptible to such a condition or disorder, and/or to treat a subject suffering from the condition or disorder.

The compounds of this invention are formulated with conventional carriers and excipients, which will be selected in accord with ordinary practice. For example, tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The manner in which the compounds are administered can vary. Oral administration is preferable but the compositions may also be administered via injection, i.e., intravenously, intramuscularly, subcutaneously, intraperitoneally, intraarterially, intrathecally; and intracerebroventricularly. Intravenous administration is the preferred method of injection. The formulations can also be administered using other means, for example, rectal administration, by inhalation (e.g., in the form of an aerosol either nasally or using delivery articles; topically (e.g., in lotion form); transdermally (e.g., using a transdermal patch) or iontophoretically; or by sublingual or buccal administration. Pharmaceutical formulations containing the active ingredient may be in any form suitable for the intended method of administration.

When intended for oral use, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of HCV infections as described below.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

The compositions of the compounds of Formula I-V can be administered intermittently or at a gradual, continuous, constant or controlled rate. In addition, the time of day and the number of times per day that the pharmaceutical formulation is administered can vary. The effective dose of an active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. The effective dose can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### HIV Combination Therapy

In an embodiment of the present invention and as will be appreciated by those skilled in the art, the compound of the present invention may be administered in combination with other therapeutic compounds. The compounds of the present invention may be employed alone or in combination with other therapeutic agents, including other compounds of the present invention. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the formulae of the present invention including salts or solvates thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The compounds of the present invention may be used in the treatment of a variety of disorders and conditions and, as such, the compounds of the present invention may be used in combination with a variety of other suitable therapeutic agents useful in the treatment or prophylaxis of those disorders or conditions.

In one embodiment, non-limiting examples of suitable combinations include combinations of one or more compounds of the present invention with one or more HIV protease inhibitors, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, CCR8 inhibitors, RNase H inhibitors, maturation inhibitors, pharmacokinetic enhancers, and other drugs for treating HIV.

More specifically, one or more compounds of the present invention may be combined with one or more compounds selected from the group consisting of

1) HIV protease inhibitors, e.g., amprenavir (Agenerase), atazanavir (Reyataz), fosamprenavir (Lexiva), indinavir (Crixivan), lopinavir, ritonavir (norvir), nelfinavir (Viracept), saquinavir (Invirase), tipranavir (Aptivus), brecanavir, darunavir (Prezista), TMC-126, TMC-114, TMC-310911, CTP-518, mozenavir (DMP-450), JE-2147 (AG1776), L-756423, RO0334649, KNI-272, DPC-681, DPC-684, DG17, GS-8374, MK-8122 (PPL-100), DG35, and AG 1859, SPI-256, TMC 52390, PL-337, SM-322377, SM-309515, GRL-02031, CRS-074, CRS-075, KB-98, and A-790742,

2) HIV non-nucleoside inhibitors of reverse transcriptase, e.g., capravirine, emivirine, delaviridine (Rescriptor), efavirenz (Sustiva), nevirapine (Viramune), (+)-calanolide A, calanolide B, etravirine (Intelence), GW5634, DPC-083, DPC-961, DPC-963, MIV-150, MIV-160, MIV-170, dapivirine (TMC-120), rilpivirine (TMC-278), BILR 355 BS, VRX 840773, UK-453061, RDEA 806, RDEA 427, RDEA 640, GSK-2248761 (IDX 899), ANX-201 (Thiovir), R-1206, LOC-dd, IQP-0410 (SJ-3366), YM-215389, YM-228855, CMX-052, and CMX-182,

3) HIV nucleoside inhibitors of reverse transcriptase, e.g., zidovudine (Retrovir), emtricitabine (Emtriva), didanosine (Videx), stavudine (Zerit), zalcitabine (Hivid), lamivudine (Epivir), abacavir (Ziagen), amdoxovir, elvucitabine (ACH 126443), alovudine (MIV-310), MIV-210, racivir (racemic FTC, PSI-5004), D-d4FC, phosphazide, fozivudine tidoxil, apricitibine (AVX754, SPD-754), GS-7340, KP-1461, AVX756, OBP-601, dioxolane thymine, TMC-254072, INK-20, PPI-801, PPI-802, MIV-410, 4′-Ed4T, B-108, and fosalvudine tidoxil (HDP 99.0003),

4) HIV nucleotide inhibitors of reverse transcriptase, e.g., tenofovir disoproxil fumarate (Viread), adefovir dipivoxil, GS-7340, and CMX 157,

5) HIV integrase inhibitors, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, raltegravir (Isentress, MK-0518), elvitegravir (GS-9137), BMS-538158, GSK364735C, BMS-707035, MK-2048, GSK-1349572 (S-349572), GSK-1265744 (S-265744), GSK-247303 (S-247303), S-1360 (GW810871), JTK-656, QNL-111, 1,5-DCQA, INH-001, INT-349, V-165, RIN-25, BFX-1001, BFX-1002, BFX-1003, RSC-1838, BCH-33040, and BA 011,

6) gp41 inhibitors, e.g., enfuvirtide (Fuzeon), sifuvirtide, MPI-451936, FB006M, A-329029, and TRI-1144,

7) CXCR4 inhibitors, e.g., AMD-070, KRH-3955 (CS-3955), AMD-9370, AMD-3451, RPI-MN, MSX-122, and POL-2438,

8) entry inhibitors, e.g., SP01A, PA-161, SPC3, TNX-355, DES6, SP-10, SP-03, CT-319, and CT-326,

9) gp120 inhibitors, e.g., BMS-488043 and its prodrugs, BlockAide/CR, KPC-2, and MNLP62,

10) G6PD and NADH-oxidase inhibitors, e.g., immunitin,

11) CCR5 inhibitors, e.g., aplaviroc, nifeviroc, vicriviroc (SCH-417690), maraviroc (Selzentry), PRO-140, PRO-542, INCB15050, INCB9471, PF-232798, UK-484900, SCH-532706, GSK-706769, TAK-652, TAK-220, ESN-196, RO-1752, ZM-688523, AMD-887, YM-370749, NIBR-1282, SCH-350634, ZM-688523, and CCR5 mAb004,

12) CCR8 inhibitors, e.g., ZK-756326,

13) RNase H inhibitors, e.g., ODN-93, and ODN-112, 14) maturation inhibitors, e.g., bevirimat (PA-457), MPI-461359, PA-040, MPC-9055 (vicecon, MPI-49839), ACH-100703, ACH-100706

15) pharmacokinetic enhancers, e.g., BAS-100, SPI-452, PF-4194477, PF-03716539, TMC-41629, TMC-589337, TMC-589354, TMC-558445, G5-9350, G5-9585, and roxythromycin,

16) other drugs for treating HIV, e.g., REP 9, SP-01 A, TNX-355, DES6, ODN-93, ODN-112, VGV-1, Ampligen, HRG214, Cytolin, VGX-410, VGX-820, KD-247, AMZ 0026, CYT 99007, A-221 HIV, HPH-116, DEBIO-025, BAY 50-4798, MDX010 (ipilimumab), PBS119, BIT-225, UBT-8147, ITI-367, AFX-400, BL-1050, GRN-139951, GRN-140665, AX-38679, RGB-340638, PPI-367, and ALG 889.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to maintain, prevent, or decrease viral load or CD4+ T cell count. The effective dose of compounds will of course differ from patient to patient, but in general includes amounts starting where desired therapeutic effects occur but below the amount where adverse effects are observed.

The compounds described herein, when employed in effective amounts in accordance with the methods described herein, can provide some degree of prevention of the progression of, amelioration of symptoms, and amelioration, to some degree, of the recurrence of HIV. Ideally, the effective dose of the compounds described herein is sufficient to provide the desired effects upon the disorder but is insufficient (i.e., is not at a high enough level) to provide undesirable side effects. Preferably, the compounds are administered at a dosage effective for treating the HIV and related disorders.

Most preferably, effective doses are at concentrations where maximal effects are observed to occur with a minimum of side effects. Typically, the effective dose of such compounds generally requires administering the compound in an amount of between about 0.001 mg/kg and 10,000 mg/kg of patient weight. The effective doses typically represent that amount administered as a single dose, or as one or more doses administered over a 24-hour period. In addition, the compositions are advantageously administered at an effective dose such that the concentration of the compound within the plasma of the patient normally maintains a sufficient level to prevent the decrease in CD4 count.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of a viral infection. The compounds and pharmaceutical compositions of the invention may be used to achieve a beneficial therapeutic or prophylactic effect, for example, in a subject with HIV, AIDS, or ARC.

### Diagnostic Uses

The compounds can be used in diagnostic compositions, such as probes, particularly when they are modified to include appropriate labels. For this purpose the compounds of the present invention most preferably are labeled with a radioactive isotopic moiety such as ¹¹C, ¹⁸F, ⁷⁶Br, ¹²³I or ¹²⁵I. The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al., incorporated herein by reference with regard to administration of such compounds. The compounds can be administered using known techniques. See, for example, U.S. Pat. No. 5,969,144 to London et al., as noted, incorporated by reference with regard to such administration. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al., herein incorporated by reference with regard to such analysis.

### Synthetic Methods

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art, but are not mentioned in greater detail.

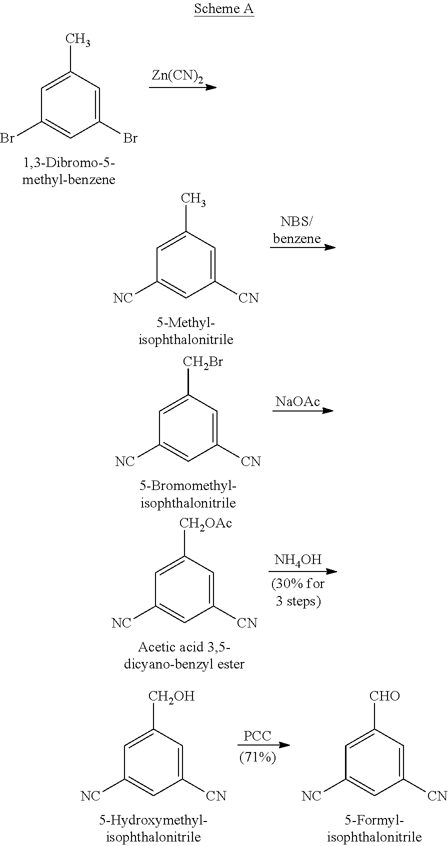

5-Methyl-isophthalonitrile: To a stirred solution of 1,3-Dibromo-5-methyl-benzene (5 g, 20 mmol) in DMF (40 ml), were added zinc cyanide (2.82 g, 24 mmol), DPPF (1.3 g, 2.34 mmol), and Pd₂ dba₃ (916 mg, 1 mmol) in this order. The flask was flushed with nitrogen and stirred in an oil bath (110-120° C.) for 22 hr. After cooling to room temperature, the mixture was evaporated in vacuo. The residue was purified by silica gel column chromatography (eluent, ethyl acetate:hexane (1:4)) to afford 1.8 g (63%) of a white solid. m.p. 178-179° C.; ¹H-NMR (200 MHz, CDCl₃) δ 2.47 (3H, s), 7.70 (2H, s), 7.76 (1H, s); m/z (EI) 142 (M⁺).
5-Hydroxymethyl-isophthalonitrile: 5-Methyl-isophthalonitrile (2.9 g, 20.4 mmol) in benzene (50 ml) was refluxed with N-bromosuccinimide (3.76 g, 21 mmol) and benzoyl peroxide (255 mg, 1.0 mmol) for 8 hr. After cooling to room temperature, the mixture was filtered and evaporated in vacuo. The residue was purified by silica gel column chromatography (eluent, ethyl acetate:hexane (1:4)) to give 2.6 g of 5-Bromomethyl-isophthalonitrile as a mixture containing starting material. This mixture was stirred with sodium acetate (3.28 g, 40 mmol) in DMF (40 ml) for overnight at room temperature. The mixture was diluted with ether, washed with water three times, dried with MgSO₄, filtered, and evaporated in vacuo to give Acetic acid 3,5-dicyano-benzyl ester. Acetic acid 3,5-dicyano-benzyl ester was stirred with ammonium hydroxide (5 ml) in methanol (50 ml). The mixture was then evaporated in vacuo and the residue was purified by silica gel column chromatography (eluent, ethyl acetate:hexane (1:1)) to afford 980 mg (30% for 3 steps) of 5-Hydroxymethyl-isophthalonitrile as a white solid. m.p. 119-120° C.; ¹H-NMR (200 MHz, CDCl₃) δ 2.19 (1H, t, J=5.2 Hz), 4.82 (2H, d, J=5.2 Hz), 7.85 (1H, s), 7.90 (2H, s); m/z (EI) 158 (M⁺).
5-Formyl-isophthalonitrile: To a stirred solution of 5-Hydroxymethyl-isophthalonitrile (0.98 g, 6.2 mmol) in dichloromethane (25 ml), were added pyridinium chlorochromate (2 g, 9.29 mmol) and celite (2 g). After stirring for 2 hr. at room temperature, the mixture was diluted with ether and filtered through a plug of silica gel. The plug was washed with ether. The combined filtrate was evaporated in vacuo and the residue was purified by silica gel column chromatography (eluent, ether:hexane (from 1:1 to 2:1)) to afford 690 mg (71%) of 5-Formyl-isophthalonitrile as a white solid. m.p. 203-204° C.; ¹H-NMR (200 MHz, CDCl₃) δ 8.19 (1H, s), 8.39 (2H, s), 10.08 (1H, s); m/z (EI) 156 (M⁺).

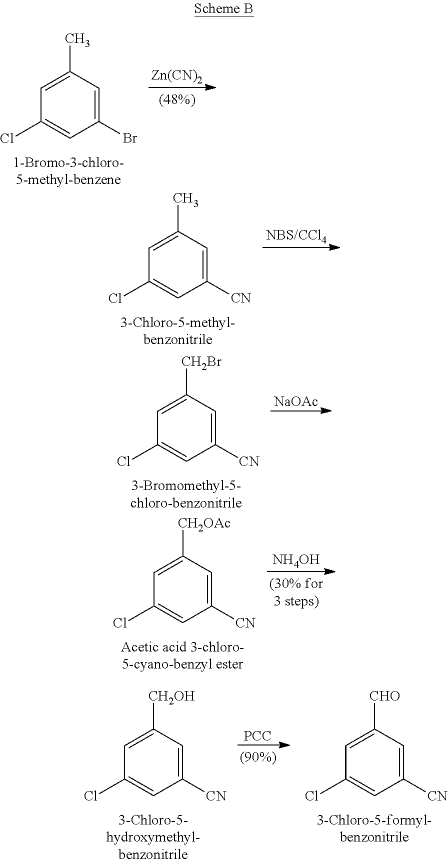

3-Chloro-5-methyl-benzonitrile: To a stirred solution of 1-Bromo-3-chloro-5-methyl-benzene (7.32 g, 35.62 mmol) in DMF (70 ml), were added zinc cyanide (2.51 g, 21.37 mmol), DPPF (395 mg, 0.712 mmol), and Pd₂ dba₃ (326 mg, 0.356 mmol) in this order. The flask was flushed with nitrogen and stirred in an oil bath (110-120° C.) for 5 hr. After cooling to room temperature, the mixture was evaporated in vacuo. The residue was purified by silica gel column chromatography (eluent, ether:hexane (from 1:19 to 1:9)) to afford 2.8 g (51%) of a pale yellow solid. m.p. 74-76° C.; ¹H-NMR (200 MHz, CDCl₃) δ 2.39 (3H, s), 7.35 (1H, s), 7.40 (1H, s), 7.44 (1H, s); m/z (EI) 151 (M⁺).
3-Chloro-5-hydroxymethyl-benzonitrile: 3-Chloro-5-methyl-benzonitrile (4.19 g, 27.64 mmol) in carbon tetrachloride (60 ml) was refluxed with N-bromosuccinimide (4.92 g, 27.64 mmol) and benzoyl peroxide (669 mg, 2.76 mmol) for 5 hr. After cooling to room temperature, the mixture was filtered and evaporated in vacuo. The residue was purified by silica gel column chromatography (eluent, ether:hexane (from 1:20 to 1:4)) to give 6.84 g of 3-Bromomethyl-5-chloro-benzonitrile as a mixture containing starting material. This mixture was stirred with sodium acetate (4.53 g, 55.28 mmol) in DMF (50 ml) for overnight at room temperature. The mixture was diluted with ether, washed with water three times, dried with MgSO₄, filtered, and evaporated in vacuo to give Acetic acid 3-chloro-5-cyano-benzyl ester. The residue was stirred with ammonium hydroxide (10 ml) in methanol (40 ml). The mixture was then evaporated in vacuo and the residue was purified by silica gel column chromatography (eluent, ethyl acetate:hexane (1:2)) to afford 1.43 g (30% for 3 steps) of 3-Chloro-5-hydroxymethyl-benzonitrile as a white solid. m.p. 110-111° C.; ¹H-NMR (200 MHz, CDCl₃) δ 2.06 (1H, t, J=5.6 Hz), 4.74 (2H, d, J=5.6 Hz), 7.55 (2H, s), 7.61 (1H, s); m/z (EI) 167 (M⁺).
3-Chloro-5-formyl-benzonitrile: To a stirred solution of 3-Chloro-5-hydroxymethyl-benzonitrile (1.43 g, 8.53 mmol) in dichloromethane (50 ml), were added pyridinium chlorochromate (2.76 g, 12.8 mmol) and celite (2.76 g). After stirring for 2 hr. at room temperature, the mixture was diluted with ether and filtered through a plug of silica gel. The plug was washed with ether. The combined filtrate was evaporated in vacuo and the residue was purified by silica gel column chromatography (eluent, ethyl acetate:hexane (1:4)) to afford 1.28 g (90%) of a white solid. m.p. 132-133° C.; ¹H-NMR (200 MHz, CDCl₃) δ 7.89 (1H, s), 8.05 (1H, s), 8.09 (1H, s), 10.02 (1H, s); m/z (EI) 165 (M⁺).

### Example 1

### 3-(5-Cyclopropylmethyl-3-isopropyl-6-methyl-2-oxo-1,2-dihydro-pyridine-4-carbonyl)-5-methyl-benzonitrile

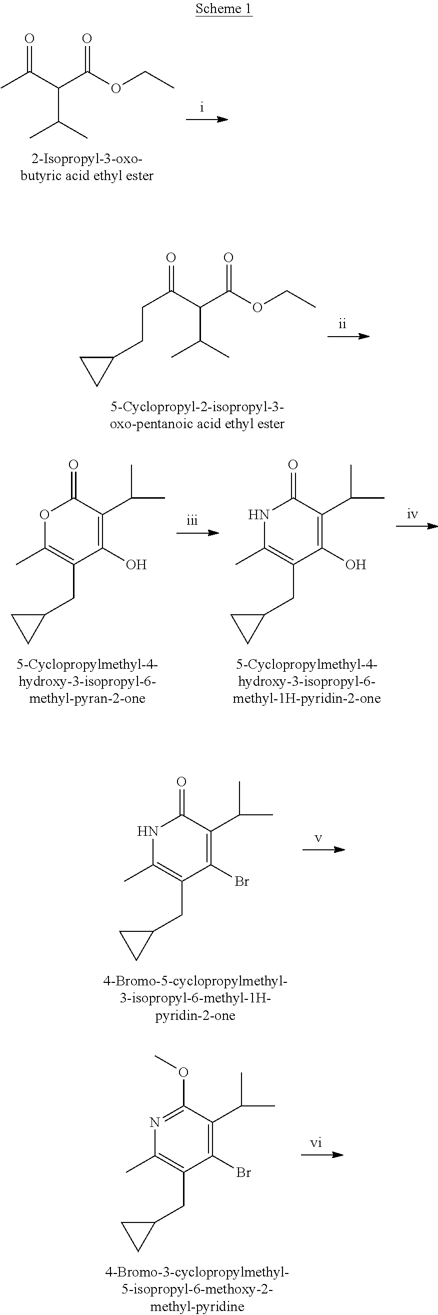

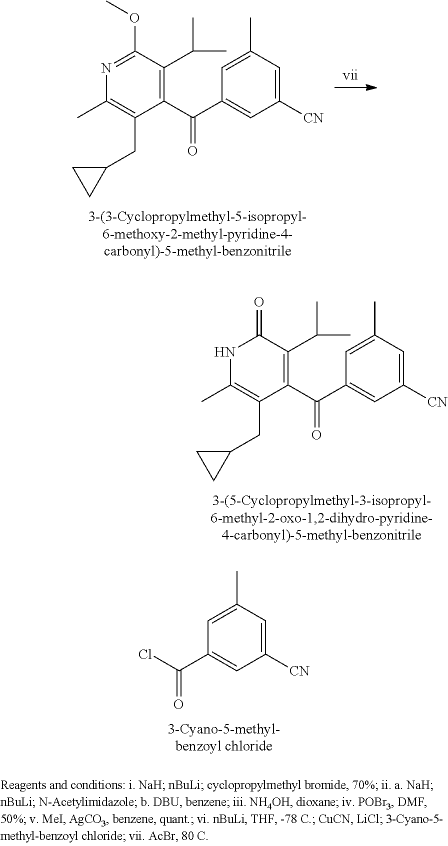

5-Cyclopropyl-2-isopropyl-3-oxo-pentanoic acid ethyl ester: To a flask containing 60% sodium hydride (0.619 g, 15.47 mmol) in THF (35 mL) at rt was added 2-Isopropyl-3-oxo-butyric acid ethyl ester (2.42 g, 14.06 mmol). Reaction mixture was stirred for 10 minutes, giving a clear solution. After cooling to 0° C., n-butyllithium (1.6 M in hexanes, 9.2 mL, 14.76 mmol) was added, giving a yellow solution. Reaction mixture was stirred for 10 minutes and cyclopropylmethyl bromide (Aldrich, 1.50 mL, 15.47 mmol) was added. Reaction mixture was stirred at rt for 2 h and then quenched with concentrated HCl, diluted with H2O and extracted with ethyl ether. The organic layer was washed with H2O, dried (MgSO₄) and concentrated. The residue was purified by flash column chromatography (1 to 10% ethyl acetate) to give a yellow oil (2.24 g, 70%). ¹H NMR (300 MHz, CDCl₃): 4.13 (q, J=7.2 Hz, 2H), 3.16 (d, J=9.6 Hz, 1H), 2.6-2.3 (m, 3H), 1.42 (q, J=7.2 Hz, 2H), 1.22 (t, J=6.9 Hz, 3H), 0.92 (d, J=6.6 Hz, 3H), 0.86 (d, J=6.6 Hz, 3H), 0.66-0.58 (m, 1H), 0.35 (q, J=5.4 Hz, 2H), −0.2 (q, J=5.4 Hz, 2H).
5-Cyclopropylmethyl-4-hydroxy-3-isopropyl-6-methyl-pyran-2-one: To a suspension of 60% sodium hydride (0.065 g, 1.63 mmol) in THF (5 mL) was added a solution of 5-Cyclopropyl-2-isopropyl-3-oxo-pentanoic acid ethyl ester (0.334 g, 1.48 mmol) in THF (1 mL). The mixture was stirred for 5 minutes and stirred for 15 minutes at 0° C. nButyllithium (1.6 M in hexane, 1.48 mL, 2.37 mmol) was added and the yellow reaction mixture was stirred for 1 h. Cooled to −78° C. and a solution of N-acetylimidazole (0.244 g, 2.22 mmol) in THF (2.5 mL) was added. Reaction was stirred for 1 h at −78° C. and quenched with concentrated HCl, diluted with H2O and extracted with ethyl ether. The organic layer was washed with H2O, dried (MgSO₄) and concentrated. Residue was dissolved in benzene (5 mL) and DBU (0.26 mL) was added. Reaction mixture was refluxed for 1 h, cooled to rt and poured into saturated NaHCO3 solution. Washed with ethyl ether (3×) and aqueous layer acidified with concentrated HCl to pH 1. Extracted with ethyl ether (3×) and combined organic layer was dried (MgSO₄) and concentrated. The residue was purified by flash column chromatography (1 to 40% ethyl acetate) to give a white film (0.1009, 31%). ¹H NMR (300 MHz, CDCl₃): 3.2-3.1 (m, 1H), 2.34 (d, J=6.3 Hz, 2H), 2.15 (s, 1H), 1.23 (s, 3H), 1.21 (s, 3H), 0.88-0.82 (m, 1H), 0.40 (q, J=5.7 Hz, 2H), 0.13 (q, J=5.4 Hz, 2H); Mass Spectrum: 223.1 (M+H), 221.0 (M−H).
5-Cyclopropylmethyl-4-hydroxy-3-isopropyl-6-methyl-1H-pyridin-2-one: A solution of 5-Cyclopropylmethyl-4-hydroxy-3-isopropyl-6-methyl-pyran-2-one (0.667 g, 3.00 mmol) in ammonium hydroxide (28-30%, 10 mL) and dioxane (7.3 mL) was heated at 120° C. in a sealed tube for 2 h. Reaction mixture was concentrated to ˜7 mL, poured into 1 M KHSO4 solution and extracted with 0-10% ethyl acetate/hexanes (3×). The combined organic layer was dried (MgSO₄), concentrated and Recrystallized from hot acetone to give a white solid (0.4577 g, 69%). ¹H NMR (300 MHz, CD₃OD): 3.3-3.2 (m, 1H), 2.48 (d, J=6.3 Hz, 2H), 2.24 (s, 1H), 1.27 (s, 3H), 1.25 (s, 3H), 0.90-0.86 (m, 1H), 0.38 (q, J=5.4 Hz, 2H), 0.12 (q, J=4.8 Hz, 2H); Mass Spectrum: 222.1 (M+H), 220.2 (M−H).
4-Bromo-5-cyclopropylmethyl-3-isopropyl-6-methyl-1H-pyridin-2-one: A solution of 5-Cyclopropylmethyl-4-hydroxy-3-isopropyl-6-methyl-1H-pyridin-2-one (0.4283 g. 1.94 mmol) and phosphorous oxybromide (0.610 g, 2.13 mmol) in DMF (7.7 mL) was heated at 80° C. for 2 h, then at 100° C. for 2 h. Reaction mixture was cooled to rt and ice was added. Mixture was stirred for 30 minutes, extracted with ethyl acetate (3×), dried (MgSO₄), and concentrated. The residue was purified by flash column chromatography (silica gel, 10 to 50% ethyl acetate/hexane) to give a white solid (0.3642 g, 66%). ¹H NMR (300 MHz, CD₃OD): 3.6-3.2 (m, 1H), 2.64 (d, J=6.3 Hz, 2H), 2.26 (s, 1H), 1.29 (s, 3H), 1.26 (s, 3H), 0.97-0.92 (m, 1H), 0.41 (q, J=4.2 Hz, 2H), 0.20 (q, J=5.78 Hz, 2H); Mass Spectrum: 284.1, 286.1 (M+H).
4-Bromo-3-cyclopropylmethyl-5-isopropyl-6-methoxy-2-methyl-pyridine: To a mixture of 4-Bromo-5-cyclopropylmethyl-3-isopropyl-6-methyl-1H-pyridin-2-one (0.2621 g, 0.922 mmol) and silver carbonate (0.763 g, 2.77 mmol) in benzene (3.0 mL) was added iodomethane (0.29 mL, 4.61 mmol). Reaction mixture was stirred at 45° C. for 16 h, then at 60° C. for 24 h. Reaction mixture was cooled to rt, filtered through a pad of Celite and washed with ethyl acetate. The filtrate was concentrated and purified by flash column chromatography (silica gel, dichloromethane) to give a colorless oil (0.275 g, 100%). ¹H NMR (300 MHz, CDCl₃): 3.88 (s, 3H), 3.6-3.5 (m, 1H), 2.75 (br s, 2H), 2.44 (s, 3H), 1.26 (s, 3H), 1.24 (s, 3H), 0.96 (br s, 1H), 0.42 (br s, 2H), 0.24 (br s, 2 H); ¹³C NMR (75 MHz, CDCl₃): 159.8, 151.2, 138.3, 127.4, 126.7, 53.0, 36.0, 32.7, 29.7, 23.1, 19.7, 10.5, 4.4; Mass Spectrum: 298.2, 300.1 (M+H).
3-Cyano-5-methyl-benzoyl chloride: A solution of 3-methoxycarbonyl-5-methylbenzoic acid (Combi-Blocks, 3.0 g, 15.45 mmol) in thionyl chloride (14 mL) was refluxed for 1 h, concentrated and co-evaporated with benzene (3×). The residue was dissolved in dichloromethane (5 mL) and added to ammonium hydroxide (28-30%, 10 mL) at 0° C., giving a white precipitate. Reaction mixture was stirred for 5 minutes at 0° C., H2O and ethyl acetate were added and the mixture was filtered to give a white solid (2.69 g). To this solid was added phosphorous oxychloride (9.3 mL) and heated to 100° C., giving a clear solution. After heating for 1 h, reaction mixture was cooled, concentrated, dissolved in dichloromethane and treated with saturated Na₂CO₃ solution. Mixture was extracted with ethyl acetate (2×), dried (MgSO₄), concentrated and purified by flash column chromatography (silica gel, dichloromethane) to give 3-Cyano-5-methyl-benzoic acid methyl ester (2.076 g, 77%).
3-Cyano-5-methyl-benzoic acid methyl ester (2.00 g, 11.42 mmol) was dissolved in methanol (40 mL) and H2O (66 mL) and 1M sodium hydroxide solution (13.8 mL) was added to give a white precipitate. Reaction mixture was gently heated to give a clear solution. Reaction mixture was acidified with 1N HCl to pH 2 and cooled to rt. The resulting white precipitate was collected by filtration and dried to give 3-Cyano-5-methyl-benzoic acid (1.487 g, 80%).
3-Cyano-5-methyl-benzoic acid (0.306 g, 1.90 mmol) was suspended in dichloromethane (4 mL) and oxalyl chloride (0.331 mL, 3.80 mmol.) and DMF (1 drop) were added at 0° C., then warmed to rt over 2 h. Reaction mixture was concentrated, co-evaporated with anhydrous toluene (3×) and used in next step without further purification.
3-(3-Cyclopropylmethyl-5-isopropyl-6-methoxy-2-methyl-pyridine-4-carbonyl)-5-methyl-benzonitrile: To a solution of 4-Bromo-3-cyclopropylmethyl-5-isopropyl-6-methoxy-2-methyl-pyridine (0.1224 g, 0.410 mmol) in THF (freshly distilled from Na/benzophenone, 5.0 mL) at −78° C. was added n-butyllithium (1.6 M in hexanes, 0.334 mL, 0.534 mmol) and stirred for 1 h. Copper(I) cyanide (0.0549 g, 0.534 mmol) and lithium chloride (0.0452 g, 1.066 mmol), dried at 80° C. for 2 h, was dissolved with sonication in freshly distilled THF (˜2 mL). This yellow solution was added to reaction mixture and stirred for 30 minutes at −78° C. 3-Cyano-5-methyl-benzoyl chloride (0.220 g, 1.23 mmol) in freshly distilled THF (˜1 mL) was added to reaction mixture, stirred for 5 minutes at −78° C., warmed to rt and stirred for 30 minutes. Reaction was quenched with brine and extracted with ethyl acetate. Organic layer was dried (MgSO₄), concentrated and purified by flash column chromatography (0 to 10 ethyl acetate) to give title product as an impure mixture (0.1545 g). ¹H NMR (300 MHz, CD₃OD): 7.81 (s, 3H), 3.92 (s, 3H), 2.45 (s, 3H), 2.39 (s, 3H), 2.4-2.3 (m, 1H), 2.34 (dd, J=15, 6.9 Hz, 1H), 2.08 (dd, J=15, 6.0 Hz, 1H), 1.13 (d, J=6.6 Hz, 3H), 1.04 (d, J=6.9 Hz, 3H), 0.8-0.7 (m, 1H), 0.3-0.2 (m, 2H), 0.1-0.05 (m, 1H), −0.15-−0.2 (m, 1H); ¹³C NMR (75 MHz, CDCl₃): 196.8, 159.7, 152.5, 147.2, 141.1, 137.6, 137.3, 133.6, 129.6, 123.3, 122.2, 117.2, 113.0, 51.2, 33.0, 30.2, 20.8, 19.6, 19.4, 18.8, 10.6, 4.7, 3.4; Mass Spectrum: 363.2 (M+H).
3-(5-Cyclopropylmethyl-3-isopropyl-6-methyl-2-oxo-1,2-dihydro-pyridine-4-carbonyl)-5-methyl-benzonitrile: A solution of 3-(3-Cyclopropylmethyl-5-isopropyl-6-methoxy-2-methyl-pyridine-4-carbonyl)-5-methyl-benzonitrile (0.1301 g, 0.359 mmol) in acetyl bromide (6.0 mL) was heated at 50° C. for 1 h, then at 80° C. for 1 h. Reaction mixture was cooled to rt and co-evaporated with acetonitrile (3×), methanol and acetonitrile to give an off-white solid. Reside was purified by flash column chromatography (silica gel, 0 to 5% methanol/dichloromethane) to give a yellow oil. Oil was dissolved in hot isopropanol (4 mL) and H2O (9 mL) was slowly added at 90° C. to give a cloudy yellow solution. Mixture was slowly cooled to rt and stirred for 3 h, resulting in an off-white solid. Solid was collected by filtration and dried under vacuum for 18 h to give title compound (95.6 mg, 76%). Melting point: 222-224° C.; ¹H NMR (300 MHz, CD₃OD): 7.91 (s, 2H), 7.84 (s, 1H), 2.43 (s, 3H), 2.4-2.3 (m, 1H), 2.30 (s, 3H), 2.16 (dd, J=15.6, 6.9 Hz, 1H), 2.00 (dd, J=14.7, 5.7 Hz, 1H), 1.18 (d, J=6.6 Hz, 3 H), 1.09 (d, J=6.6 Hz, 3H), 0.7-0.6 (m, 1H), 0.3-0.2 (m, 2H), 0.03-−0.05 (m, 1H), −0.13-−0.16 (m, 1H); Mass Spectrum: 349.3 (M+H), 347.2 (M−H).

### Example 2

### 5-(5-Cyclopropylmethyl-3-isopropyl-6-methyl-2-oxo-1,2-dihydro-pyridine-4-carbonyl)-isophthalonitrile

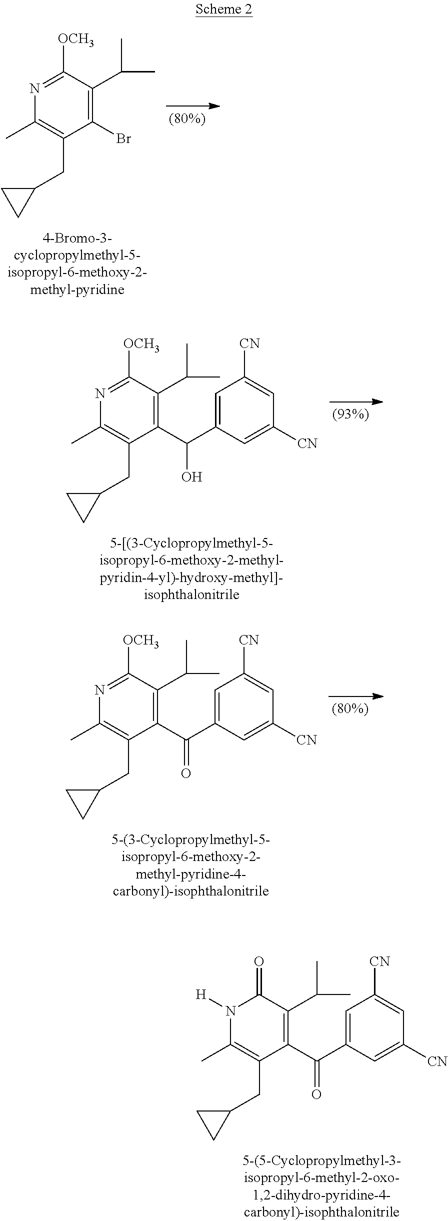

5-[(3-Cyclopropylmethyl-5-isopropyl-6-methoxy-2-methyl-pyridin-4-yl)-hydroxy-methyl]-isophthalonitrile: To a stirred solution of 4-Bromo-3-cyclopropylmethyl-5-isopropyl-6-methoxy-2-methyl-pyridine (298 mg, 1 mmol) in THF (8 ml) cooled in a dry ice-acetone bath (−78° C.), was added n-butyllithium (1.6M in hexane, 0.6 ml, 0.96 mmol). After stirring for 1 hr., 3,5-dicyanobenzaldehyde (172 mg, 1.1 mmol) in THF (2 ml) was added. The mixture was stirred at below −75° C. for 50 min. and then stirred at room temperature for 10 min. Saturated aq. NH₄Cl solution (10 ml) was added and the product was extracted with ethyl acetate, dried (MgSO₄), filtered, and evaporated in vacuo. The residue was purified by silica gel column chromatography (eluent, ethyl acetate:hexane (1:9)) to afford 300 mg (80%) of a white foam. ¹H-NMR (200 MHz, CDCl₃) δ 0.14-0.21 (2H, m), 0.45-0.76 (6H, m), 1.21 (3H, d, J=6.4 Hz), 2.48-2.55 (4H, m), 2.66 (2H, d, J=6.0 Hz), 2.84-2.91 (1H, m), 3.92 (3H, s), 6.24 (1H, d, J=5.8 Hz), 7.81 (3H, s); m/z (EI) 375 (M⁺).
5-(3-Cyclopropylmethyl-5-isopropyl-6-methoxy-2-methyl-pyridine-4-carbonyl)-isophthalonitrile: To a stirred solution of 5-[(3-Cyclopropylmethyl-5-isopropyl-6-methoxy-2-methyl-pyridin-4-yl)-hydroxy-methyl]-isophthalonitrile (280 mg, 0.7457 mmol) in DMF (2 ml), was added pyridinium dichromate (421 mg, 1.118 mmol). The mixture was stirred at room temperature for 15 hr. The mixture was evaporated in vacuo and the residue was purified by silica gel column chromatography (eluent, ethyl acetate:hexane (1:15)) to afford 260 mg (93%) of a white foam. ¹H-NMR (200 MHz, CDCl₃) δ −0.18-0.08 (2H, m), 0.25-0.39 (2H, m), 0.66-0.72 (1H, m), 1.07 (3H, d, J=7.0 Hz), 1.20 (3H, d, J=7.0 Hz), 2.08 (1H, dd, J=15.0 Hz, 5.6 Hz), 2.27-2.49 (2H, m), 2.53 (3H, s), 3.99 (3H, s), 8.12 (1H, s), 8.24 (2H, s); m/z (EI) 373 (M⁺).
5-(5-Cyclopropylmethyl-3-isopropyl-6-methyl-2-oxo-1,2-dihydro-pyridine-4-carbonyl)-isophthalonitrile: A solution of 5-(3-Cyclopropylmethyl-5-isopropyl-6-methoxy-2-methyl-pyridine-4-carbonyl)-isophthalonitrile (230 mg, 0.6158 mmol) in acetyl bromide (6 ml) was stirred in an oil bath (100-110° C.) for 2 hr. Reaction mixture was cooled to rt and co-evaporated with acetonitrile three times, methanol, and acetonitrile. The residue was purified by silica gel column chromatography (eluent, 2% methanol in dichloromethane) to give 179 mg (80%) of a yellow film. The product was recrystallized from ether to afford a yellow crystal. m.p. 266-267° C.; ¹H-NMR (200 MHz, CDCl₃) δ −0.15-0.07 (2H, m), 0.26-0.41 (2H, m), 0.58-0.64 (1H, m), 1.19 (3H, d, J=6.6 Hz), 1.31 (3H, d, J=6.6 Hz), 2.00 (1H, dd, J=15.8 Hz, 6.0 Hz), 2.14 (1H, dd, J=15.8 Hz, 6.0 Hz), 2.27-2.37 (1H, m), 2.43 (3H, s), 8.16 (1H, s), 8.35 (2H, s), 13.26 (1H, s); m/z (EI) 359 (M⁺).

### Example 3

### 3-Chloro-5-(5-cyclopropylmethyl-3-isopropyl-6-methyl-2-oxo-1,2-dihydro-pyridine-4-carbonyl)-benzonitrile

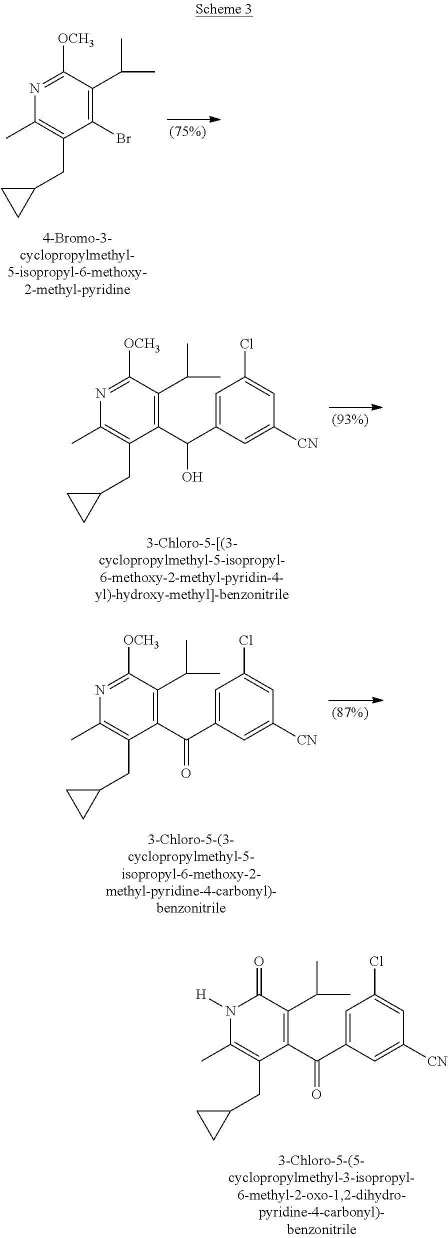

3-Chloro-5-[(3-cyclopropylmethyl-5-isopropyl-6-methoxy-2-methyl-pyridin-4-yl)-hydroxy-methyl]-benzonitrile: To a stirred solution of 4-Bromo-3-cyclopropylmethyl-5-isopropyl-6-methoxy-2-methyl-pyridine (298 mg, 1 mmol) in THF (8 ml) cooled in a dry ice-acetone bath (−78° C.), was added n-butyllithium (1.6M in hexane, 0.6 ml, 0.96 mmol). After stirring for 1 hr., 3-chloro-5-cyanobenzaldehyde (182 mg, 1.1 mmol) in THF 2 ml) was added. The mixture was stirred at below −75° C. for 1 hr. and then stirred at room temperature for 10 min. Saturated aq. NH₄Cl solution (10 ml) was added and the product was extracted with ethyl acetate, dried (MgSO₄), filtered, and evaporated in vacuo. The residue was purified by silica gel column chromatography (eluent, ethyl acetate:hexane (1:15)) to afford 290 mg (75%) of a white foam. ¹H-NMR (200 MHz, CDCl₃) δ 0.18-0.225 (2H, m), 0.44-0.51 (2H, m), 0.68-0.88 (4H, m), 1.21 (3H, d, J=6.8 Hz), 2.42 (1H, d, J=4.6 Hz), 2.54 (3H, s), 2.65 (2H, d, J=6.2 Hz), 2.91-3.01 (1H, m), 3.91 (3H, s), 6.21 (1H, d, J=4.6 Hz), 7.45 (1H, s), 7.50 (1H, s), 7.52 (1H, s); m/z (EI) 384 (M⁺).
3-Chloro-5-(3-cyclopropylmethyl-5-isopropyl-6-methoxy-2-methyl-pyridine-4-carbonyl)-benzonitrile: To a stirred solution of 3-Chloro-5-[(3-cyclopropylmethyl-5-isopropyl-6-methoxy-2-methyl-pyridin-4-yl)-hydroxy-methyl]-benzonitrile (247 mg, 0.64 mmol) in DMF (2 ml), was added pyridinium dichromate (362 mg, 0.96 mmol). The mixture was stirred at room temperature for 17 hr. The mixture was evaporated in vacuo and the residue was purified by silica gel column chromatography (eluent, ethyl acetate:hexane (1:20)) to afford 230 mg (93%) of a white foam. ¹H-NMR (200 MHz, CDCl₃) δ −0.14-0.08 (2H, m), 0.28-0.39 (2H, m), 0.62-0.78 (1H, m), 1.09 (3H, d, J=6.8 Hz), 1.20 (3H, d, J=6.8 Hz), 2.11 (1H, dd, J=15.0 Hz, 5.6 Hz), 2.27-2.47 (2H, m), 2.52 (3H, s), 3.98 (3H, s), 7.83 (1H, s), 7.84 (1H, s), 8.02 (1H, s); m/z (EI) 382 (M⁺).
3-Chloro-5-(5-cyclopropylmethyl-3-isopropyl-6-methyl-2-oxo-1,2-dihydro-pyridine-4-carbonyl)-benzonitrile: A solution of 3-Chloro-5-(3-cyclopropylmethyl-5-isopropyl-6-methoxy-2-methyl-pyridine-4-carbonyl)-benzonitrile (203 mg, 0.53 mmol) in acetyl bromide (6 ml) was stirred in an oil bath (100-110° C.) for 2.5 hr. Reaction mixture was cooled to rt and co-evaporated with acetonitrile three times, methanol, and acetonitrile. The residue was purified by silica gel column chromatography (eluent, 2% methanol in dichloromethane) to give 170 mg (87%) of a pale yellow solid. The product was recrystallized from chloroform-ether to afford a pale yellow crystal. m.p. 237-238° C.; ¹H-NMR (200 MHz, CDCl₃) δ −0.16-0.06 (2H, m), 0.26-0.40 (2H, m), 0.59-0.66 (1H, m), 1.21 (3H, d, J=6.8 Hz), 1.31 (3H, d, J=6.8 Hz), 2.02 (1H, dd, J=15.0 Hz, 5.2 Hz), 2.14 (1H, dd, J=15.0 Hz, 5.2 Hz), 2.31-2.37 (1H, m), 2.42 (3H, s), 7.87 (1H, s), 8.01 (1H, s), 8.10 (1H, s), 13.38 (1H, s); m/z (EI) 368 (M⁺).

### Example 4

### Acetic acid 1-[4-(3-cyano-5-methyl-benzoyl)-5-isopropyl-2-methyl-6-oxo-1,6-dihydro-pyridin-3-ylmethyl]-cyclopropylmethyl ester and

### Example 5

### 3-[5-(1-Hydroxymethyl-cyclopropylmethyl)-3-isopropyl-6-methyl-2-oxo-1,2-dihydro-pyridine-4-carbonyl]-5-methyl-benzonitrile

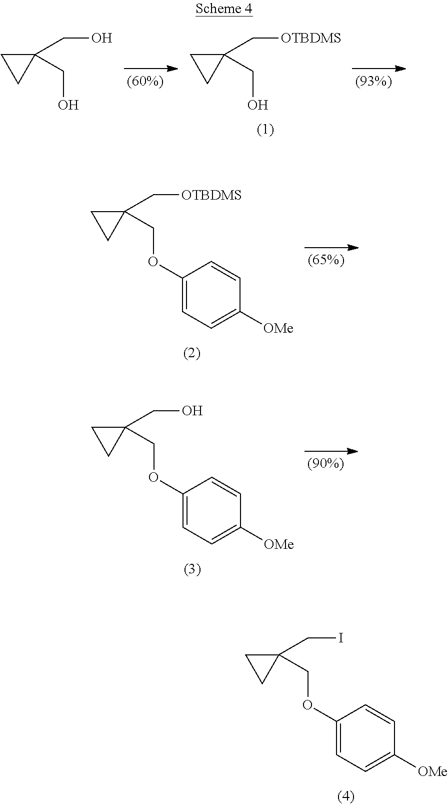

### [1-(tert-Butyl-dimethyl-silanyloxymethyl)-cyclopropyl]-methanol (1)

To an ice-cold solution of (1-hydroxymethyl-cyclopropyl)-methanol (10.19 g, 99.77 mmol) in DMF (200 ml), were added imidazole (10.19 g, 149 mmol) and tert-butyldimethylsilyl chloride (15 g, 99.77 mmol). After stirring for 1 hr., the mixture was diluted with ether, washed with water, dried with MgSO₄, filtered, and concentrated. The residue was purified by silica gel column chromatography (eluent, ethyl acetate:hexane (from 1:9 to 1:4)) to afford 11.9 g (60%) of the title compound as a colorless oil.

¹H-NMR (200 MHz, CDCl₃) δ 0.06 (6H, s), 0.41-0.55 (4H, m), 0.90 (9H, s), 2.78 (1H, t, J=5.8 Hz), 3.56 (2H, d, J=5.8 Hz), 3.61 (2H, s).

### tert-Butyl-[1-(4-methoxy-phenoxymethyl)-cyclopropylmethoxy]-dimethyl-silane (2)

To an ice-cold solution of [1-(tert-butyl-dimethyl-silanyloxymethyl)-cyclopropyl]-methanol (14 g, 69.18 mmol), triphenylphosphine (23.5 g, 89.67 mmol), p-methoxyphenol (25.7 g, 207 mmol) in dichloromethane (270 ml), was dropwise added 40 wt. % diethyl azodicarboxylate (40.8 ml, 89.67 mmol). After stirring for 10 min., the mixture was further stirred at room temperature for overnight. The mixture was then concentrated and the residue was purified by silica gel column chromatography (eluent, ether:hexane (from 1:15 to 1:9)) to afford 21 g (94%) of the title compound as a colorless oil.

¹H-NMR (300 MHz, CDCl₃) δ 0.01 (6H, s), 0.52-0.53 (4H, m), 0.84 (9H, s), 3.61 (2H, s), 3.74 (3H, s), 3.79 (2H, s), 6.80-6.81 (4H, m).

m/z (EI) 322 (M⁺)

### [1-(4-Methoxy-phenoxymethyl)-cyclopropyl]-methanol (3)

To a stirred solution of tert-butyl-[1-(4-methoxy-phenoxymethyl)-cyclopropylmethoxy]-dimethyl-silane (22.7 g, 70.38 mmol) in THF (60 ml) in water bath, was added tetrabutylammonium fluoride (1 M in THF, 71 ml, 71 mmol). The mixture was then stirred at room temperature for 1 hr. and concentrated. The residue was purified by silica gel column chromatography (eluent, ether:hexane (1:1)) to afford 9.62 g (65%) of the title compound as a white solid.

m.p. 80-81° C.

¹H-NMR (200 MHz, CDCl₃) δ 0.62 (4H, s), 2.09 (1H, t, J=0.52 Hz), 3.63 (2H, d, J=0.52 Hz), 3.76 (3H, s), 3.88 (2H, s), 6.83 (4H, s).

m/z (EI) 208 (M⁺)

### 1-(1-Iodomethyl-cyclopropylmethoxy)-4-methoxy-benzene (4)

To a stirred solution of [1-(4-methoxy-phenoxymethyl)-cyclopropyl]-methanol (9.62 g, 46.19 mmol), triphenylphosphine (14.54 g, 55.43 mmol), and imidazole (3.77 g, 55.43 mmol) in DMF (90 ml) at −30° C., was portionwise added iodine (12.89 g, 50.81 mmol). With stirring, the mixture was gradually warmed up to 5° C. during 1 hr. The mixture was diluted with ether, washed with water, dried with MgSO₄, filtered, and concentrated. The residue was purified by silica gel column chromatography (eluent, 3% ether in hexane) to afford 12.9 g (87%) of the title compound as a white solid.

m.p. 53-54° C.

¹H-NMR (300 MHz, CDCl₃) δ 0.72-0.76 (2H, m), 0.97-1.01 (2H, m), 3.41 (2H, s), 3.77 (3H, s), 3.84 (2H, s), 6.81-6.88 (4H, m).

m/z (EI) 318 (M⁺)

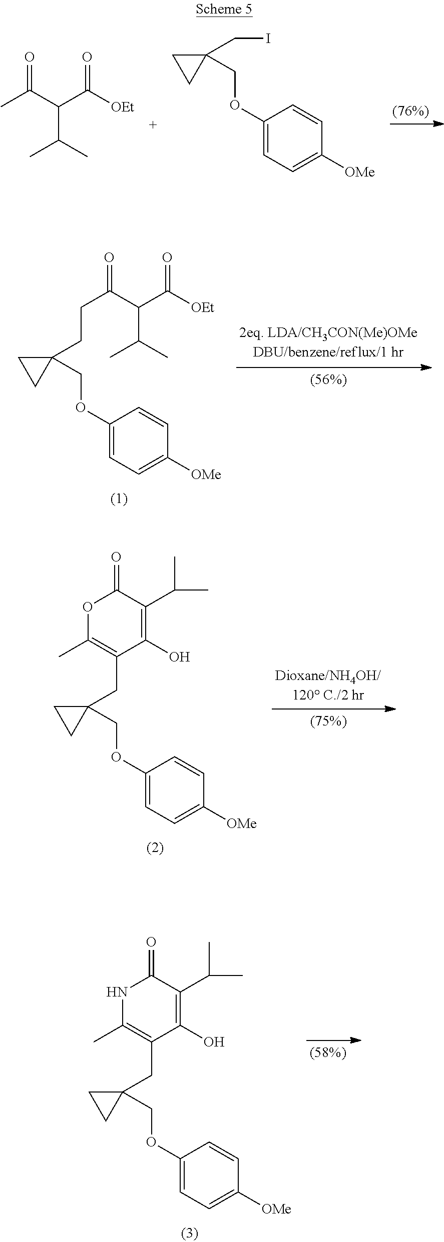

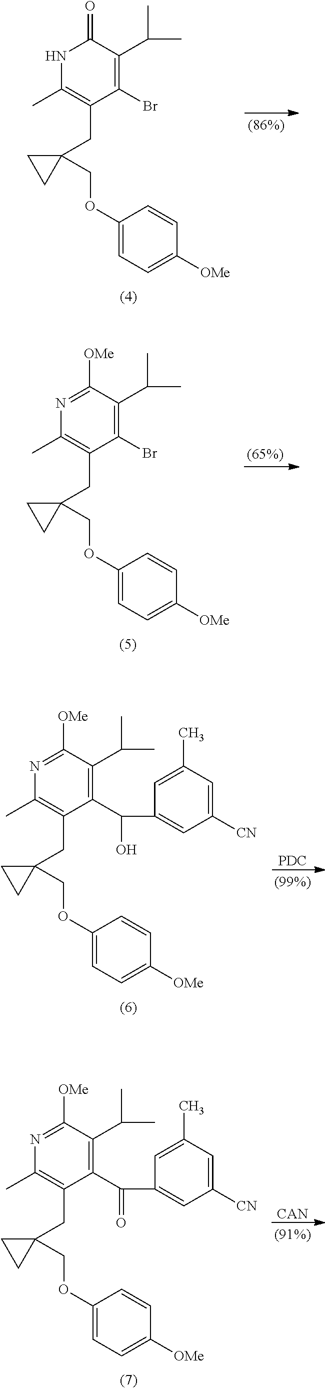

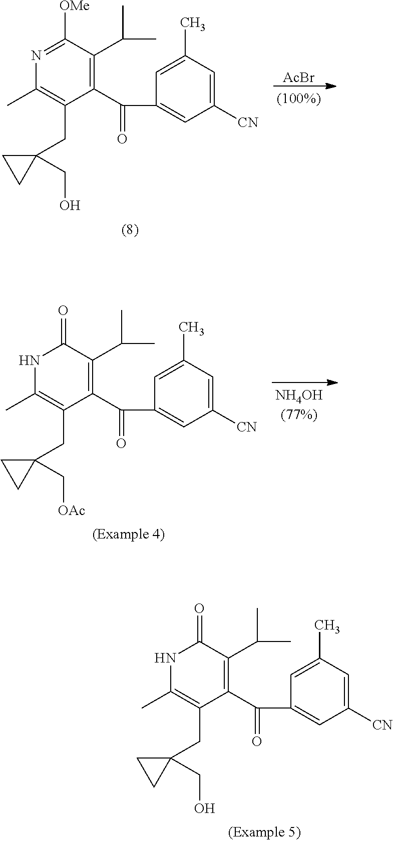

### 2-Isopropyl-5-[1-(4-methoxy-phenoxymethyl)-cyclopropyl]-3-oxo-pentanoic acid ethyl ester(1)

To a flask containing 60% sodium hydride (0.528 g, 13.2 mmol) in THF (22 mL) at room temperature, was added a solution of 2-Isopropyl-3-oxo-butyric acid ethyl ester (2.06 g, 11.96 mmol) in THF (5 ml). Reaction mixture was stirred for 30 min., giving a clear solution. After cooling to −20° C., n-butyllithium (1.6 M in hexane, 8 mL, 12.8 mmol) was added, giving a yellow solution. Reaction mixture was stirred for 2 hr. at −5-0° C. After cooling to −10° C., a solution of 1-(1-iodomethyl-cyclopropylmethoxy)-4-methoxy-benzene (4.18 g, 13.14 mmol) in THF (5 ml) was added. After 30 min., reaction mixture was stirred at room temperature for 3 hr. and then quenched with concentrated HCl, diluted with water and extracted with ethyl ether. The organic layer was washed with water, dried with MgSO₄, filtered, and concentrated. The residue was purified by silica gel column chromatography (eluent, ether:hexane (1:9)) to give 3.2 g (74%) of the title compound as a colorless oil.

¹H NMR (300 MHz, CDCl₃) □ 0.40-0.55 (4H, m), 0.92 (3H, d, J=6.6 Hz), 0.96 (3H, d, J=6.6 Hz), 1.22 (3H, t, J=7.0 Hz), 1.72-1.79 (2H, m), 2.37-2.45 (1H, m), 2.66-2.74 (2H, m), 3.20 (1H, d, J=9.4 Hz), 3.67 (2H, s), 3.76 (3H, s), 4.14 (2H, q, J=7.0 Hz), 6.82 (4H, s).

m/z (EI) 362 (M⁺)

### 4-Hydroxy-3-isopropyl-5-[1-(4-methoxy-phenoxymethyl)-cyclopropylmethyl]-6-methyl-pyran-2-one(2)

To a solution of diisobutylamine (3.72 ml, 21.3 mmol) in THF (80 mL) at −78° C., was added n-butyllithium(1.6M in hexane, 13.4 ml, 21.4 mmol). With stirring, the mixture was warmed up to −10° C. during 2 hr. A solution of 2-isopropyl-5-[1-(4-methoxy-phenoxymethyl)-cyclopropyl]-3-oxo-pentanoic acid ethyl ester (3.2 g, 8.83 mmol) in THF (20 ml) was then added dropwise and the stirring was continued for 2 hr. at −5˜0° C. A neat solution of N-methoxy-N-methyl-acetamide (1.14 ml, 10.7 mmol) was then added and the mixture was stirred for 40 min. at −5-0° C. The mixture was further stirred at room temperature for 1 hr. and concentrated. The residue was quenched with 2N HCl (54 ml) and the product was extracted with ether. The organic layer was washed with brine, dried with MgSO₄, filtered, and concentrated. Residue was dissolved in benzene (40 mL) and DBU (2 mL, 13.37 mmol) was added. Reaction mixture was refluxed for 1 hr., cooled to room temperature and poured into saturated NaHCO₃ solution. The product was extracted with ether (3×) and aqueous layer was acidified with concentrated HCl to pH 1. The aqueous layer was extracted with ether (3×). The combined organic layer was dried with MgSO₄ and concentrated. The residue was purified by silica gel column chromatography (eluent, ethyl acetate:hexane (from 1:4 to 1:3)) to give 1.79 g (56%) of the title compound as a white syrup.

¹H NMR (200 MHz, CDCl₃) □ 0.50-0.65 (4H, m), 1.26 (6H, d, J=7.0 Hz), 2.16 (3H, s), 2.78 (2H, s), 3.18-3.32 (1H, s), 3.73 (2H, s), 3.78 (3H, s), 6.86 (4H, s), 7.86 (1H, s). m/z (EI) 358 (M⁺)

### 4-Hydroxy-3-isopropyl-5-[1-(4-methoxy-phenoxymethyl)-cyclopropylmethyl]-6-methyl-1H-pyridin-2-one (3)

A solution of 4-hydroxy-3-isopropyl-5-[1-(4-methoxy-phenoxymethyl)-cyclopropylmethyl]-6-methyl-pyran-2-one (5.28 g, 14.73 mmol) in ammonium hydroxide (28˜30%, 49 mL) and dioxane (37 mL) was heated at 120° C. in a steel bomb for 2 hr. Reaction mixture was concentrated, poured into 1 M KHSO₄ solution (150 ml) and extracted with ethyl acetate (3×). The combined organic layer was dried with MgSO₄, filtered, concentrated, and recrystallized from hot acetone to give 3.95 g (75%) of the title compound as a white solid.

m.p. 222-223° C.

¹H NMR (300 MHz, DMSO-d₆) □0.36-0.39 (4H, m), 1.19 (6H, d, J=6.8 Hz), 2.03 (3H, s), 2.72 (2H, s), 3.14-3.19 (1H, m), 3.65 (2H, s), 3.68 (3H, s), 6.83 (4H, s), 8.81 (1H, s), 10.75 (1H, s). m/z (EI) 357 (M⁺)

### 4-Bromo-3-isopropyl-5-[1-(4-methoxy-phenoxymethyl)-cyclopropylmethyl]-6-methyl-1H-pyridin-2-one(4)

A solution of 4-hydroxy-3-isopropyl-5-[1-(4-methoxy-phenoxymethyl)-cyclopropylmethyl]-6-methyl-1H-pyridin-2-one (2.2 g, 6.15 mmol) and phosphorous oxybromide (2.12 g, 7.38 mmol) in DMF (12 mL) was heated at 100° C. for 2 hr. Reaction mixture was cooled to room temperature and ice was added. Mixture was stirred for 30 minutes, extracted with ethyl acetate (3×), dried with MgSO₄, filtered, and concentrated. The residue was purified by silica gel column chromatography (eluent, ethyl acetate:hexane (from 1:4 to ethyl acetate)) to give 1.5 g (58%) of the title compound as a yellow solid.

m.p. 165-166° C.

¹H NMR (300 MHz, CDCl₃) □ 0.31-0.35 (2H, m), 0.45-0.49 (2H, m), 1.33 (6H, d, J=6.9 Hz), 2.35 (3H, s), 3.15 (2H, s), 3.47-3.54 (1H, m), 3.76 (2H, s), 3.79 (3H, s), 6.82 (4H, s), 12.45 (1H, s). m/z (EI) 419 (M⁺), 421 (M+2⁺)

### 4-Bromo-3-isopropyl-2-methoxy-5-[1-(4-methoxy-phenoxymethyl)-cyclopropylmethyl]-6-methyl-pyridine (5)

To a mixture of 4-bromo-3-isopropyl-5-[1-(4-methoxy-phenoxymethyl)-cyclopropylmethyl]-6-methyl-1H-pyridin-2-one (2.16 g, 5.14 mmol) and silver carbonate (4.25 g, 15.41 mmol) in benzene (20 mL) was added iodomethane (1.6 mL, 25.69 mmol). Reaction mixture was stirred at 50-60° C. for 20 hr. After cooling to room temperature, the mixture was filtered through a pad of celite and washed with ethyl acetate. The filtrate was concentrated and purified by silica gel column chromatography (eluent, 3% ether in hexane) to give 1.93 g (86%) of the title compound as a colorless oil.

¹H NMR (300 MHz, CDCl₃) □ 0.26-0.28 (2H, m), 0.42-0.43 (2H, m), 1.25 (6H, d, J=7.0 Hz), 2.47 (3H, s), 3.29 (2H, s), 3.55-3.65 (1H, m), 3.77 (3H, s), 3.79 (2H, s), 3.89 (3H, s), 6.83 (4H, s). m/z (EI) 433 (M⁺), 435 (M+2⁺)

### 3-(Hydroxy-{3-isopropyl-2-methoxy-5-[1-(4-methoxy-phenoxymethyl)-cyclopropylmethyl]-6-methyl-pyridin-4-yl}-5-methyl-benzonitrile (6)

To a stirred solution of 4-bromo-3-isopropyl-2-methoxy-5-[1-(4-methoxy-phenoxymethyl)-cyclopropylmethyl]-6-methyl-pyridine (1.15 g, 2.64 mmol) in THF (24 ml) cooled in a dry ice-acetone bath (−78° C.), was added n-butyllithium(1.6M in hexane, 1.6 ml, 2.56 mmol). After stirring for 1 hr., a solution of 3-cyano-5-methylbenzaldehyde (481 mg, 3.31 mmol) in THF (8 ml) was added. The mixture was stirred at −78° C. for 50 min. and then stirred at room temperature for 10 min. Saturated aq. NH₄Cl solution (10 ml) was added and the product was extracted with ethyl acetate, dried with MgSO₄, filtered, and concentrated. The residue was purified by silica gel column chromatography(eluent, ethyl acetate:hexane (1:4)) to afford 870 mg(65%) of the title compound as a white solid.

m.p. 162-163° C.

¹H-NMR (300 MHz, CDCl₃) δ 0.43-0.59 (4H, m), 0.66 (3H, d, J=6.9 Hz), 1.18 (3H, d, J=6.9 Hz), 2.33 (3H, s), 2.53 (3H, s), 2.95-3.05 (3H, m), 3.12 (1H, d, J=15.1 Hz), 3.67 (2H, t, J=9.6 Hz), 3.74 (3H, s), 3.90 (3H, s), 6.33 (1H, d, J=5.0 Hz), 6.65-6.78 (4H, m), 7.31 (1H, s), 7.32 (1H, s), 7.33 (1H, s). m/z (EI) 500 (M⁺)

### 3-{3-Isopropyl-2-methoxy-5-[1-(4-methoxy-phenoxymethyl)-cyclopropyl methyl]-6-methyl-pyridine-4-carbonyl}-5-methyl-benzonitrile (7)

To a stirred solution of 3-(hydroxy-{3-isopropyl-2-methoxy-5-[1-(4-methoxy-phenoxymethyl)-cyclopropylmethyl]-6-methyl-pyridin-4-yl}-5-methyl-benzonitrile (870 mg, 1.74 mmol) in DMF (10 ml), was added pyridinium dichromate (980 mg, 2.61 mmol). The mixture was stirred at room temperature for 16 hr. The mixture was evaporated in vacuo and the residue was purified by silica gel column chromatography(eluent, ethyl acetate:hexane (1:9)) to afford 860 mg(99%) of the title compound as a white foam.

¹H-NMR (300 MHz, CDCl₃) δ 0.11-0.19 (1H, m), 0.30-0.35 (1H, m), 0.38-0.42 (1H, m), 0.48-0.52 (1H, m), 1.06 (3H, d, J=6.8 Hz), 1.18 (3H, d, J=6.8 Hz), 2.38-2.46 (5H, m), 2.52 (3H, s), 2.98 (1H, d, J=14.6 Hz), 3.51 (1H, d, J=9.4 Hz), 3.64 (1H, d), J=9.4 Hz), 3.74 (3H, s), 3.97 (3H, s), 6.69-6.79 (4H, m), 7.65 (1H, s), 7.74 (1H, s), 7.82 (1H, s).

m/z (EI) 498 (M⁺)

### 3-[3-(1-Hydroxymethyl-cyclopropylmethyl)-5-isopropyl-6-methoxy-2-methyl-pyridine-4-carbonyl]-5-methyl-benzonitrile (8)

To an ice-cold solution of 3-{3-isopropyl-2-methoxy-5-[1-(4-methoxy-phenoxymethyl)-cyclopropylmethyl]-6-methyl-pyridine-4-carbonyl}-5-methyl-benzonitrile (860 mg, 1.7248 mmol) in acetonitrile (24 ml), were added ammonium cerium(IV) nitrate (2.27 g, 4.1395 mmol) and water (6 ml) in this order. After stirring for 10 min., the mixture was diluted with ethyl acetate, washed with brine and saturated NaHCO₃ solution, dried with MgSO₄, filtered, and concentrated. The residue was purified by silica gel column chromatography(eluent, ethyl acetate:hexane (1:4)) to afford 620 mg(91%) of the title compound as a pale yellow syrup.

¹H-NMR (200 MHz, CDCl₃) δ 0.1-0.51 (4H, m), 1.05 (3H, d, J=6.8 Hz), 1.19 (3H, d, J=6.8 Hz), 1.74 (1H, t, J=5.2 Hz), 2.35-2.49 (7H, m), 2.70 (1H, d, J=14.6 Hz), 3.14 (1H, dd, J=11.4 Hz, 7.4 Hz), 3.38 (1H, dd, J=11.4 Hz, 7.4 Hz), 3.98 (3H, s), 7.67 (1H, s), 3.72 (1H, s), 3.85 (1H, s).

m/z (EI) 392 (M⁺)

### Acetic acid 1-[4-(3-cyano-5-methyl-benzoyl)-5-isopropyl-2-methyl-6-oxo-1,6-dihydro-pyridin-3-ylmethyl]-cyclopropylmethyl ester (Example 4)

A solution of 3-[3-(1-hydroxymethyl-cyclopropylmethyl)-5-isopropyl-6-methoxy-2-methyl-pyridine-4-carbonyl]-5-methyl-benzonitrile (120 mg, 0.3057 mmol) in acetyl bromide (4 mL) was heated at 100° C. for 2 hr. Reaction mixture was cooled to room temperature and co-evaporated with acetonitrile three times, methanol, and acetonitrile. The residue was purified by silica gel column chromatography(eluent, ethyl acetate:hexane (1:4)) to afford 130 mg(100%) of the title compound as a colorless syrup.

¹H-NMR (200 MHz, CDCl₃) δ 0.14-0.51 (4H, m), 1.18 (3H, d, J=6.6 Hz, 1.27 (3H, d, J=6.6 Hz), 2.01 (3H, s), 2.44-2.48 (7H, m), 2.65 (1H, d, J=15.0 Hz), 3.69 (1H, d, J=11.8 Hz), 3.85 (1H, d, J=11.8 Hz), 7.70 (1H, s), 7.87 (1H, s), 7.89 (1H, s), 13.28 (1H, s).

### 3-[5-(1-Hydroxymethyl-cyclopropylmethyl)-3-isopropyl-6-methyl-2-oxo-1,2-dihydro-pyridine-4-carbonyl]-5-methyl-benzonitrile

### Example 5

A solution of acetic acid 1-[4-(3-cyano-5-methyl-benzoyl)-5-isopropyl-2-methyl-6-oxo-1,6-dihydro-pyridin-3-ylmethyl]-cyclopropylmethyl ester (130 mg, 0.3057 mmol) in methanol (6 mL) was stirred with ammonium hydroxide (1 ml) at room temperature for overnight and concentrated. The reside was purified by silica gel column chromatography (eluent, ethyl acetate) to give 90 mg(77%) of the title compound as a pale yellow syrup. Recrystallization from methanol-ether-hexane resulted a white solid.

m.p. 225-226° C.

¹H-NMR (300 MHz, CDCl₃) δ 0.15-0.45 (4H, m), 1.17 (3H, d, J=6.8 Hz), 1.29 (3H, d, J=6.8 Hz), 2.29-2.59 (9H, m), 3.16 (1H, d, J=11.4 Hz), 3.57 (1H, d, J=11.4 Hz), 7.70 (1H, s), 7.87 (1H, s), 7.90 (1H, s), 13.23 (1H, s). m/z (EI) 378 (M⁺)

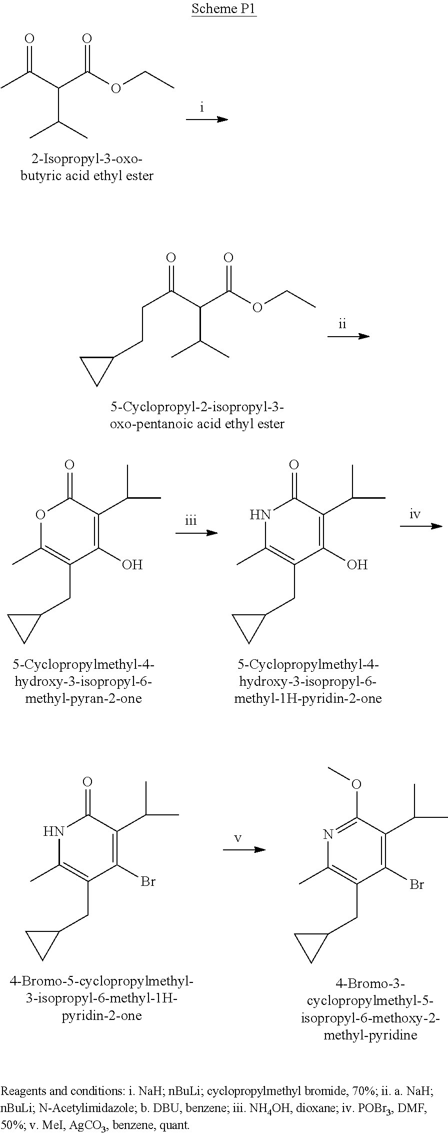

5-Cyclopropyl-2-isopropyl-3-oxo-pentanoic acid ethyl ester: To a flask containing 60% sodium hydride (0.619 g, 15.47 mmol) in THF (35 mL) at rt was added 2-Isopropyl-3-oxo-butyric acid ethyl ester (2.42 g, 14.06 mmol). Reaction mixture was stirred for 10 minutes, giving a clear solution. After cooling to 0° C., n-butyllithium (1.6 M in hexanes, 9.2 mL, 14.76 mmol) was added, giving a yellow solution. Reaction mixture was stirred for 10 minutes and cyclopropylmethyl bromide (Aldrich, 1.50 mL, 15.47 mmol) was added. Reaction mixture was stirred at rt for 2 h and then quenched with concentrated HCl, diluted with H2O and extracted with ethyl ether. The organic layer was washed with H2O, dried (MgSO₄) and concentrated. The residue was purified by flash column chromatography (1 to 10% ethyl acetate) to give a yellow oil (2.24 g, 70%). ¹H NMR (300 MHz, CDCl₃): 4.13 (q, J=7.2 Hz, 2H), 3.16 (d, J=9.6 Hz, 1H), 2.6-2.3 (m, 3H), 1.42 (q, J=7.2 Hz, 2H), 1.22 (t, J=6.9 Hz, 3H), 0.92 (d, J=6.6 Hz, 3H), 0.86 (d, J=6.6 Hz, 3H), 0.66-0.58 (m, 1H), 0.35 (q, J=5.4 Hz, 2H), −0.2 (q, J=5.4 Hz, 2H).
5-Cyclopropylmethyl-4-hydroxy-3-isopropyl-6-methyl-pyran-2-one: To a suspension of 60% sodium hydride (0.065 g, 1.63 mmol) in THF (5 mL) was added a solution of 5-Cyclopropyl-2-isopropyl-3-oxo-pentanoic acid ethyl ester (0.334 g, 1.48 mmol) in THF (1 mL). The mixture was stirred for 5 minutes and stirred for 15 minutes at 0° C. nButyllithium (1.6 M in hexane, 1.48 mL, 2.37 mmol) was added and the yellow reaction mixture was stirred for 1 h. Cooled to −78° C. and a solution of N-acetylimidazole (0.244 g, 2.22 mmol) in THF (2.5 mL) was added. Reaction was stirred for 1 h at −78° C. and quenched with concentrated HCl, diluted with H2O and extracted with ethyl ether. The organic layer was washed with H2O, dried (MgSO₄) and concentrated. Residue was dissolved in benzene (5 mL) and DBU (0.26 mL) was added. Reaction mixture was refluxed for 1 h, cooled to rt and poured into saturated NaHCO3 solution. Washed with ethyl ether (3×) and aqueous layer acidified with concentrated HCl to pH 1. Extracted with ethyl ether (3×) and combined organic layer was dried (MgSO₄) and concentrated. The residue was purified by flash column chromatography (1 to 40% ethyl acetate) to give a white film (0.1009, 31%). ¹H NMR (300 MHz, CDCl₃): 3.2-3.1 (m, 1H), 2.34 (d, J=6.3 Hz, 2H), 2.15 (s, 1H), 1.23 (s, 3 H), 1.21 (s, 3H), 0.88-0.82 (m, 1H), 0.40 (q, J=5.7 Hz, 2H), 0.13 (q, J=5.4 Hz, 2H);

Mass Spectrum: 223.1 (M+H), 221.0 (M−H).

5-Cyclopropylmethyl-4-hydroxy-3-isopropyl-6-methyl-1H-pyridin-2-one: A solution of 5-Cyclopropylmethyl-4-hydroxy-3-isopropyl-6-methyl-pyran-2-one (0.667 g, 3.00 mmol) in ammonium hydroxide (28-30%, 10 mL) and dioxane (7.3 mL) was heated at 120° C. in a sealed tube for 2 h. Reaction mixture was concentrated to −7 mL, poured into 1 M KHSO4 solution and extracted with ethyl acetate (3×). The combined organic layer was dried (MgSO₄), concentrated and Recrystallized from hot acetone to give a white solid (0.4577 g, 69%). ¹H NMR (300 MHz, CD₃OD): 3.3-3.2 (m, 1H), 2.48 (d, J=6.3 Hz, 2H), 2.24 (s, 1H), 1.27 (s, 3H), 1.25 (s, 3H), 0.90-0.86 (m, 1H), 0.38 (q, J=5.4 Hz, 2H), 0.12 (q, J=4.8 Hz, 2H); Mass Spectrum: 222.1 (M+H), 220.2 (M−H).
4-Bromo-5-cyclopropylmethyl-3-isopropyl-6-methyl-1H-pyridin-2-one: A solution of 5-Cyclopropylmethyl-4-hydroxy-3-isopropyl-6-methyl-1H-pyridin-2-one (0.4283 g. 1.94 mmol) and phosphorous oxybromide (0.610 g, 2.13 mmol) in DMF (7.7 mL) was heated at 80° C. for 2 h, then at 100° C. for 2 h. Reaction mixture was cooled to rt and ice was added. Mixture was stirred for 30 minutes, extracted with ethyl acetate (3×), dried (MgSO₄), and concentrated. The residue was purified by flash column chromatography (silica gel, 10 to 50% ethyl acetate/hexane) to give a white solid (0.3642 g, 66%). ¹H NMR (300 MHz, CD₃OD): 3.6-3.2 (m, 1H), 2.64 (d, J=6.3 Hz, 2H), 2.26 (s, 1H), 1.29 (s, 3H), 1.26 (s, 3H), 0.97-0.92 (m, 1H), 0.41 (q, J=4.2 Hz, 2H), 0.20 (q, J=5.78 Hz, 2H); Mass Spectrum: 284.1, 286.1 (M+H).
4-Bromo-3-cyclopropylmethyl-5-isopropyl-6-methoxy-2-methyl-pyridine: To a mixture of 4-Bromo-5-cyclopropylmethyl-3-isopropyl-6-methyl-1H-pyridin-2-one (0.2621 g, 0.922 mmol) and silver carbonate (0.763 g, 2.77 mmol) in benzene (3.0 mL) was added iodomethane (0.29 mL, 4.61 mmol). Reaction mixture was stirred at 45° C. for 16 h, then at 60° C. for 24 h. Reaction mixture was cooled to rt, filtered through a pad of Celite and washed with ethyl acetate. The filtrate was concentrated and purified by flash column chromatography (silica gel, dichloromethane) to give a colorless oil (0.275 g, 100%). ¹H NMR (300 MHz, CDCl₃): 3.88 (s, 3H), 3.6-3.5 (m, 1H), 2.75 (br s, 2H), 2.44 (s, 3H), 1.26 (s, 3H), 1.24 (s, 3H), 0.96 (br s, 1H), 0.42 (br s, 2H), 0.24 (br s, 2 H); ¹³C NMR (75 MHz, CDCl₃): 159.8, 151.2, 138.3, 127.4, 126.7, 53.0, 36.0, 32.7, 29.7, 23.1, 19.7, 10.5, 4.4; Mass Spectrum: 298.2, 300.1 (M+H).

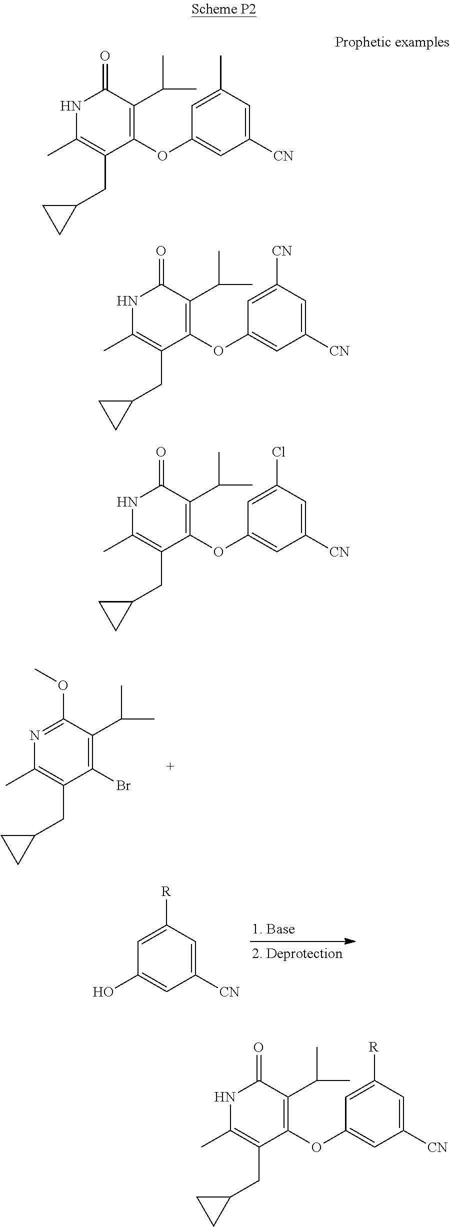

Examples in the pyridinone aryl ether class shown in Scheme P2 can be prepared by reaction of 4-Bromo-3-cyclopropylmethyl-5-isopropyl-6-methoxy-2-methyl-pyridine and the appropriate phenol under base catalysis, followed by deprotection under acidic conditions (eg acetyl bromide) to give the desired compound as shown.

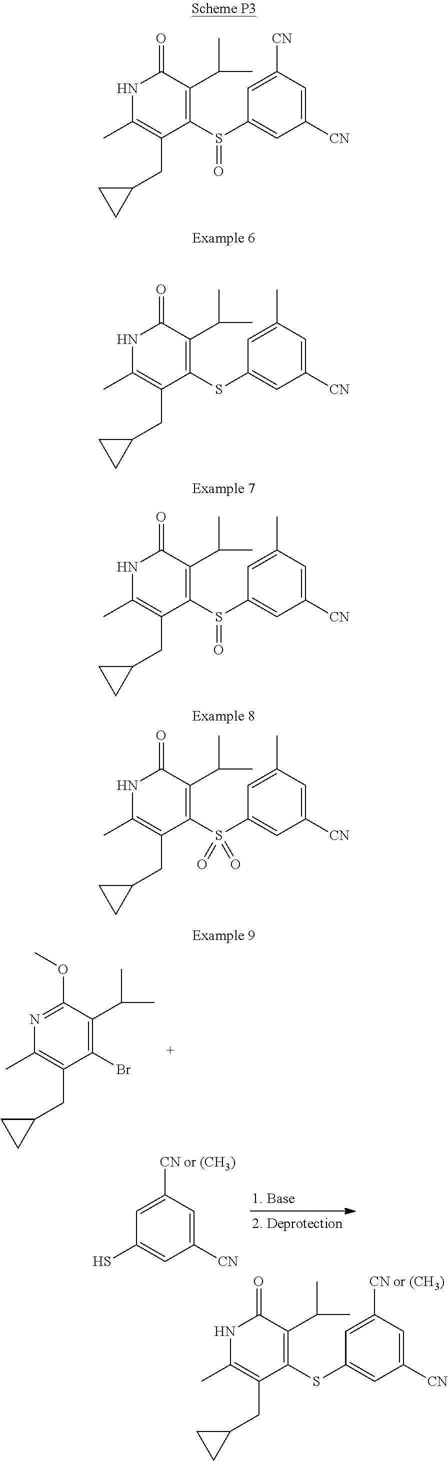

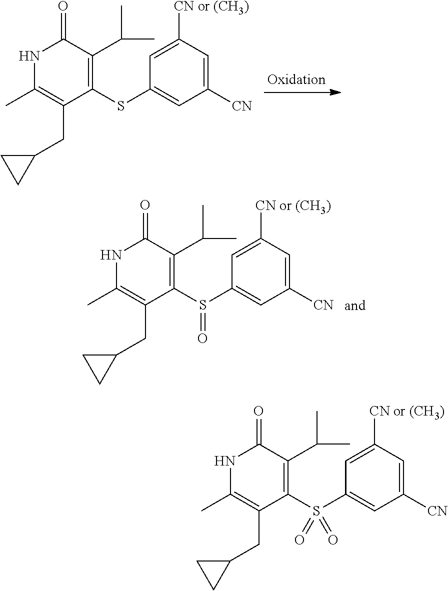

Examples 6-9 in the pyridinone aryl thioether class were prepared by reaction of 4-Bromo-3-cyclopropylmethyl-5-isopropyl-6-methoxy-2-methyl-pyridine and the appropriate thiophenol under base catalysis, followed by deprotection under acidic conditions (eg acetyl bromide) to give the desired compound, or as described below. The corresponding sulfoxide and sulfone analogs were prepared under oxidizing conditions (eg, mCPBA) or as described below.

### Example 6

### 5-(5-Cyclopropylmethyl-3-isopropyl-6-methyl-2-oxo-1,2-dihydro-pyridin-4-sulfinyl)-isophthalonitrile

### Example 17

### 5-(5-Cyclopropylmethyl-3-isopropyl-6-methyl-2-oxo-1,2-dihydro-pyridin-ylsulfanyl)-isophthalonitrile

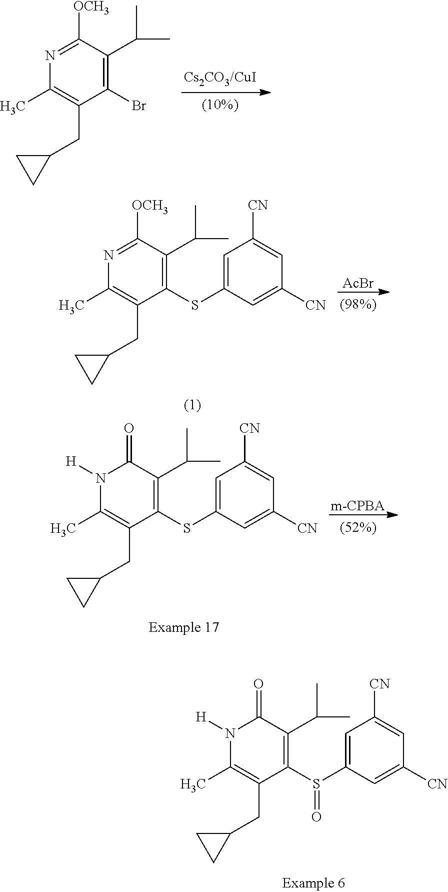

### 1) 5-(3-cyclopropylmethyl-5-isopropyl-6-methoxy-2-methyl-pyridin-4-ylsulfanyl)-isophthalonitrile (1)

A mixture of 4-bromo-3-cyclopropylmethyl-5-isopropyl-6-methoxy-2-methyl-pyridine (1.19 g, 4 mmol), 3,5-dicyanobenzenethiol (320 mg, 2 mmol), cesium carbonate (782 mg, 2.4 mmol), and copper iodide (114 mg, 0.6 mmol) in DMF (10 ml) was stirred in an oil bath (110-120° C.) for 22 hr. After cooling to room temperature, the mixture was concentrated under reduced pressure. The residue was purified by silica gel column chromatography(eluent, ether:hexane (from 1:19 to 1:4)) to afford 80 mg(10%) of the title compound as a colorless syrup.

¹H-NMR (300 MHz, CDCl₃) δ −0.05-0.02 (2H, m), 0.19-0.22 (2H, m), 0.61-0.65 (1H, m), 0.96 (6H, d, J=7.0 Hz), 2.38 (3H, s), 2.53 (2H, d, J=6.2 Hz), 3.34-3.39 (1H, m), 3.79 (3H, s), 7.16 (2H, s), 7.42 (1H, s).

m/z (EI) 377 (M⁺)

### Example 17

### 5-(5-Cyclopropylmethyl-3-isopropyl-6-methyl-2-oxo-1,2-dihydro-pyridin-ylsulfanyl)-isophthalonitrile

5-(3-cyclopropylmethyl-5-isopropyl-6-methoxy-2-methyl-pyridin-4-ylsulfanyl)-isophthalonitrile (80 mg, 0.22 mmol) was stirred with acetyl bromide (3 ml) in an oil bath (100° C.) for 2 hr. Reaction mixture was cooled to rt and co-evaporated with acetonitrile twice, methanol, and acetonitrile. The residue was purified by silica gel column chromatography(eluent, 2% methanol in dichloromethane) to give 76 mg(98%) of the title compound as a colorless syrup.

¹H-NMR (200 MHz, CDCl₃/CD₃OD) δ −0.02-0.17 (2H, m), 0.34-0.44 (2H, m), 0.73-0.83 (1H, m), 1.25 (6H, d, J=7.0 Hz), 2.44 (3H, s), 2.51 (2H, d, J=6.2 Hz), 3.49-3.60 (1H, m), 7.46 (2H, s), 7.64 (1H, s), 13.60 (1H, s).

m/z (EI) 363 (M⁺)

### Example 6

### 5-(5-Cyclopropylmethyl-3-isopropyl-6-methyl-2-oxo-1,2-dihydro-pyridin-4-sulfinyl)-isophthalonitrile

5-(5-Cyclopropylmethyl-3-isopropyl-6-methyl-2-oxo-1,2-dihydro-pyridin-ylsulfanyl)-isophthalonitrile (76 mg, 0.201 mmol) was stirred with m-chloroperbenzoic acid(<77%, 139 mg, 0.805 mmol) in dichloromethane (4 ml) at 0° C. After 3 hr., the mixture was evaporated in vacuo and the residue was purified by silica gel column chromatography(eluent, from ether:hexane (2:1) to 5% methanol in dichloromethane) to afford 40 mg(52%) of the title compound as a white solid.

m.p. 200-201° C.

¹H-NMR (200 MHz, CDCl₃/CD₃OD) δ 0.20-0.30 (2H, m), 0.48-0.82 (6H, m), 1.35 (3H, d, J=6.2 Hz), 2.39 (3H, s), 2.45-2.90 (2H, m), 3.3-3.6 (1H, m), 8.02 (2H, s), 8.12 (1H, s).

m/z (EI) 379 (M⁺)

### Example 7

### 3-(5-(cyclopropylmethyl)-3-isopropyl-6-methyl-2-oxo-1,2-dihydropyridin-4-ylthio)-5-methylbenzonitrile

### Example 8

### 3-(5-(cyclopropylmethyl)-3-isopropyl-6-methyl-2-oxo-1,2-dihydropyridin-4-ylsulfinyl)-5-methylbenzonitrile

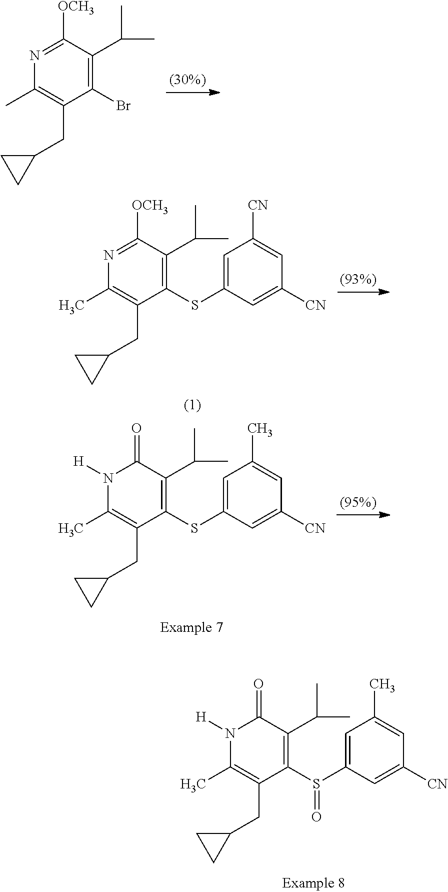

### Compound 1

A mixture of 4-bromo-3-cyclopropylmethyl-5-isopropyl-6-methoxy-2-methyl pyridine (298 mg, 1 mmol), 3-cyano-5-methylbenzenethiol (179 mg, 1.2 mmol), cesium carbonate (391 mg, 1.2 mmol), and copper iodide (57 mg, 0.3 mmol) in DMF (5 ml) was stirred in an oil bath (120-130° C.) for 24 hr. After cooling to room temperature, the mixture was concentrated under reduced pressure. The residue was purified by silica gel column chromatography(eluent, ether:hexane (1:19)) to afford 110 mg(30%) of compound 1 as a colorless syrup.

¹H-NMR (200 MHz, CDCl₃) δ 0.15-0.23 (2H, m), 0.33-0.42 (2H, m), 0.79-0.91 (1H, m), 1.12 (6H, d, J=7.4 Hz), 2.29 (3H, s), 2.55 (3H, s), 2.75 (2H, d, J=6.2 Hz), 3.57-3.71 (1H, m), 3.96 (3H, s), 6.89 (1H, s), 7.03 (1H, s), 7.16 (1H, s).

m/z (EI) 366 (M⁺)

### Example 7

Compound 1 (100 mg, 0.2728 mmol) was stirred with acetyl bromide (4 ml) in an oil bath (90-100° C.) for 2 hr. After cooling to room temperature, the mixture was coevaporated with acetonitrile twice, with methanol, and with acetonitrile. The residue was purified by silica gel column chromatography (eluent, from ethyl acetate:hexane (1:4) to ethyl acetate) to afford 90 mg (93%) of Example 7 as a white solid.

m.p. 179-180° C.

¹H-NMR (200 MHz, CDCl₃) δ 0.12-0.19 (2H, m), 0.35-0.44 (2H, m), 0.77-0.88 (1H, m), 1.25 (6H, d, J=7.0 Hz), 2.32 (3H, s), 2.43 (3H, s), 2.56 (2H, d, J=6.2 Hz), 3.55-3.65 (1H, m), 7.05 (1H, s), 7.10 (1H, s), 7.20 (1H, s), 13.45 (1H, br. s).

m/z (EI) 352 (M⁺)

### Example 8

A mixture of Example 7 (67 mg, 0.19 mmol) and <77% 3-chloroperbenzoic acid (170 mg, ˜0.75 mmol) in dichloromethane (5 ml) was stirred in an ice bath for 3 hr. The mixture was evaporated in vacuo and the residue was purified by silica gel column chromatography(eluent, from ether:hexane (2:1) to ethyl acetate:hexane (2:1)) to afford 67 mg(95%) of Example 8 as a white solid.

m.p. 230-231° C.

¹H-NMR (200 MHz, CDCl₃) δ 0.20-0.30 (2H, m), 0.44-0.48 (2H, m), 0.60-0.90 (4H, m), 1.38 (3H, d, J=6.8 Hz), 2.42 (3H, s), 2.46 (3H, s), 2.48-2.59 (1H, m), 2.79 (1H, dd, J=15.4 Hz, 5.4 Hz), 3.53-3.64 (1H, m), 7.49 (1H, s), 7.50 (1H, s), 7.62 (1H, s), 13.22 (1H, br. s).

m/z (CI) 369 (M+H⁺)

### Preparation of 3-Cyano-5-methylbenzenethiol

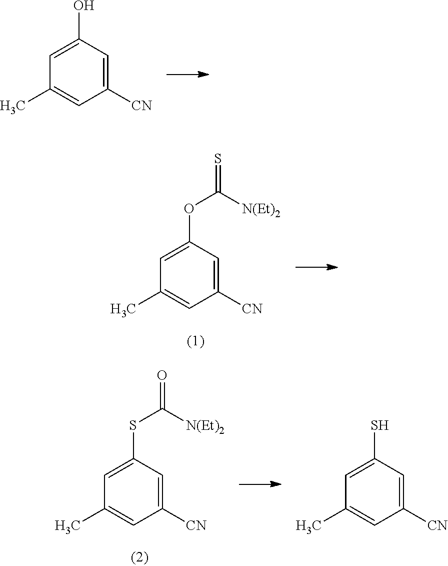

### Diethyl-thiocarbamic acid O-(3-cyano-5-methylphenyl) ester(1)

To a stirred solution of 60% sodium hydride (342 mg, 8.5 mmol) in 1-methyl-2-pyrrolidinone (3 ml) in an ice bath, was added a solution of 3-cyano-5-methylphenol (879 mg, 6.6 mmol) [WO 02/085860] in 1-methyl-2-pyrrolidinone (4 ml). After 10 min., ice bath was removed and a solution of diethylthiocarbamoyl chloride (1.297 g, 8.5 mmol) in 1-methyl-2-pyrrolidinone (5 ml) was added. The mixture was stirred for 30 min at room temperature and at 75° C. for 2 hr. After cooling to room temperature, the mixture was diluted with water (30 ml) and extracted with ethyl acetate (30 ml) three times. The combined organic solution was washed with brine, dried with MgSO₄, filtered, and concentrated in vacuo. The residue was purified by silica gel column chromatography(eluent, from hexane to ether:hexane (1:9)) to give 1.34 g (81%) of the title compound as a pale brown oil.

¹H-NMR (200 MHz, CDCl₃) δ 1.33 (6H, t, J=7.4 Hz), 2.41 (3H, s), 3.69 (2H, q, J=7.2 Hz), 3.89 (2H, q, J=7.0 Hz), 7.13 (1H, s), 7.18 (1H, s), 7.34 (1H, s).

m/z (EI) 248 (M⁺)

### Diethyl-thiocarbamic acid S-(3-cyano-5-methylphenyl) ester(2)

Diethyl-thiocarbamic acid O-(3-cyano-5-methylphenyl) ester (1.34 g, 5.395 mmol) was heated in an oil bath (190-200° C.) for 22 hr to give a brown solid. The solid was purified by silica gel column chromatography(eluent, from hexane to ethyl acetate:hexane (1:4)) to give 1.0 g (74%) of the title compound as a white solid.

m.p. 89-90° C.

¹H-NMR (200 MHz, CDCl₃) δ 1.22-1.40 (6H, br. s), 2.40 (3H, s), 3.42 (4H, q, J=7.0 Hz), 7.46 (1H, s), 7.55 (1H, s), 7.61 (1H, s).

m/z (EI) 248 (M⁺)

### 3-Cyano-5-methylbenzenethiol

Diethyl-thiocarbamic acid S-(3-cyano-5-methylphenyl) ester (1.6 g, 6.44 mmol) in methanol (8 ml) was stirred with sodium hydroxide (773 mg, 19.32 mmol) for 16 hr at room temperature. The mixture was concentrated in vacuo and the residue was dissolved in 1N sodium hydroxide solution (18 ml) and stirred at room temperature for 30 min. The mixture was washed with dichloromethane (20 ml) twice and ether (20 ml). The aqueous layer was cooled in an ice bath and acidified with 2N HCl (20 ml). The product was extracted with dichloromethane (40 ml) twice, ether (20 ml), and ethyl acetate (20 ml). The combined organic solution was washed with brine, dried with MgSO₄, filtered, and concentrated in vacuo. The residue was purified by silica gel column chromatography(eluent, from ether:hexane (1:9) to ethyl acetate:hexane (1:1)) to give 810 mg(84%) of the title compound as a white solid.

m.p. 90-91° C.

¹H-NMR (200 MHz, CDCl₃) δ 2.34 (3H, s), 3.53 (1H, s), 7.24 (1H, s), 7.29 (1H, s), 7.34 (1H, s)

m/z (EI) 149 (M⁺)

### Example 9

### 3-(5-Cyclopropylmethyl-3-isopropyl-6-methyl-2-oxo-1,2-dihydro-pyridine-4-sulfonyl)-5-methyl-benzonitrile

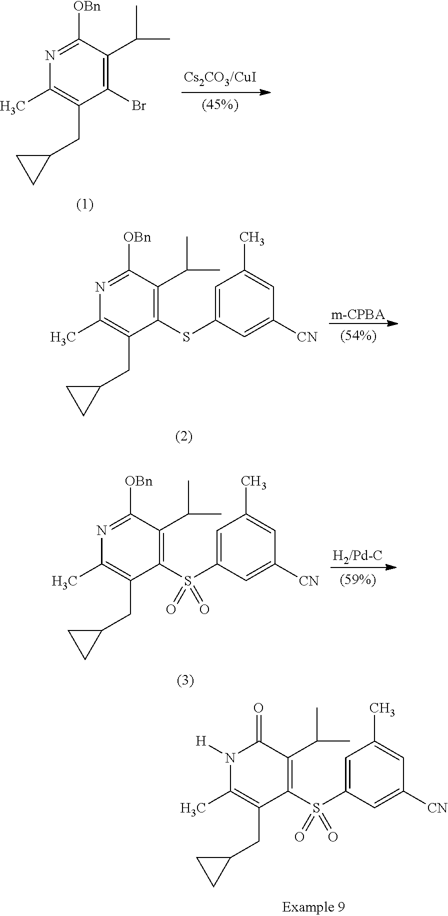

### 2-Benzyloxy-4-bromo-5-cyclopropylmethyl-3-isopropyl-6-methyl-pyridine (1)

To a stirred solution of 4-bromo-5-cyclopropylmethyl-3-isopropyl-6-methyl-1H-pyridine-2-one (4.26 g, 15 mmol) and silver carbonate (12.4 g, 45 mmol) in benzene (50 ml), was added benzyl bromide (2.68 ml, 22.5 mmol). The mixture was then stirred for 24 hr. at 100-110° C. (oil bath) in the dark. After cooling to room temperature, the mixture was filtered through a celite pad and the pad was washed with benzene. The combined filtrate was concentrated in vacuo and the residue was stirred with ammonium hydroxide (40 ml) in methanol (100 ml) for 30 min. at room temperature. The mixture was then evaporated in vacuo and the residue was purified by silica gel column chromatography(eluent, hexane) to afford 4.03 g (72%) of the title compound as a colorless oil.

¹H-NMR (200 MHz, CDCl₃) δ 0.24-0.49 (4H, m), 0.97-1.10 (1H, m), 1.29 (6H, d, J=7.0 Hz), 2.48 (3H, s), 2.77 (2H, d, J=6.4 Hz), 3.63 (1H, m), 5.38 (2H, s), 7.23-7.49 (5H, m).

m/z (EI) 374 (M⁺), 376 (M+2⁺)

### 3-(2-Benzyloxy-5-cyclopropylmethyl-3-isopropyl-6-methyl-pyridin-4-ylsulfanyl)-5-methyl-benzonitrile (2)

A mixture of 2-benzyloxy-4-bromo-5-cyclopropylmethyl-3-isopropyl-6-methyl-pyridine (1.12 g, 3 mmol), 3-cyano-5-methylbenzenethiol (537 mg, 3.6 mmol), cesium carbonate (1.17 mg, 3.6 mmol), and copper iodide (171 mg, 0.9 mmol) in DMF (10 ml) was stirred in an oil bath (110-120° C.) for 16 hr. After cooling to room temperature, the mixture was concentrated under reduced pressure. The residue was purified by silica gel column chromatography(eluent, ether:hexane (1:19)) to afford 600 mg(45%) of the title compound as a colorless syrup.

¹H-NMR (200 MHz, CDCl₃) δ 0.19-0.23 (2H, m), 0.36-0.40 (2H, m), 0.85 (1H, m), 1.14 (6H, d, J=7.0 Hz), 2.29 (3H, s), 2.56 (3H, s), 2.76 (2H, d, J=6.6 Hz), 3.65 (1H, m), 5.43 (2H, s), 6.90 (1H, s), 7.03 (1H, s), 7.16 (1H, s), 7.30-7.51 (5H, m).

m/z (EI) 442 (M⁺)

### 3-(2-Benzyloxy-5-cyclopropylmethyl-3-isopropyl-6-methyl-pyridine-4-sulfonyl)-5-methyl-benzonitrile (3)

3-(2-Benzyloxy-5-cyclopropylmethyl-3-isopropyl-6-methyl-pyridin-4-ylsulfanyl)-5-methyl-benzonitrile (477 mg, 1.077 mmol) was stirred with m-chloroperbenzoic acid(<77%, 966 mg, 4.31 mmol) in chloroform (20 ml) at room temperature. After 24 hr., the mixture was evaporated in vacuo and the residue was purified by silica gel column chromatography(eluent, ether:hexane (1:9)) to afford 280 mg(54%) of the title compound as a colorless syrup.

¹H-NMR (200 MHz, CDCl₃) δ 0.29-0.54 (4H, m), 0.91-1.07 (7H, m), 2.45 (3H, s), 2.63 (3H, s), 3.24 (2H, d, J=6.4 Hz), 3.52-3.65 (1H, m), 5.39 (2H, s), 7.29-7.46 (5H, m), 7.62 (1H, s), 7.76 (1H, s), 7.80 (1H, s).

m/z (EI) 474 (M⁺)

### Example 9

### 3-(5-Cyclopropylmethyl-3-isopropyl-6-methyl-2-oxo-1,2-dihydro-pyridine-4-sulfonyl)-5-methyl-benzonitrile

3-(2-Benzyloxy-5-cyclopropylmethyl-3-isopropyl-6-methyl-pyridine-4-sulfonyl)-5-methyl-benzonitrile (339 mg, 0.714 mmol) was stirred with 10% palladium/carbon (88 mg) in ethanol (20 ml) under an atmosphere of hydrogen at room temperature. After 2 hr., the mixture was filtered through celite pad and the filtrate was evaporated in vacuo. The residue was purified by silica gel column chromatography(eluent, ether:hexane (from 1:2 to ether)) to afford 164 mg(59%) of the title compound as a white solid.

m.p. 246-247° C.

¹H-NMR (200 MHz, CDCl₃) δ 0.32-0.54 (4H, m), 0.91-1.05 (7H, m), 2.45 (3H, s), 2.63 (3H, s), 3.24 (2H, d, J=6.0 Hz), 3.52-3.66 (1H, m), 5.39 (2H, s), 7.29-7.45 (5H, m), 7.62 (1H, s), 7.77 (1H, s), 7.80 (1H, s).

m/z (EI) 384 (M⁺)

The following prophetic compound may be prepared similar to Example 9.

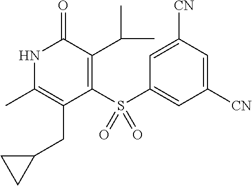

### Example 17

### 3-Cyano-5-(5-cyclopropylmethyl-3-isopropyl-6-methyl-2-oxo-1,2-dihydro-pyridine-4-carbonyl)-benzoic acid methyl ester

### Example 10

### 3-Cyano-5-(5-cyclopropylmethyl-3-isopropyl-6-methyl-2-oxo-1,2-dihydro-pyridine-4-carbonyl)-benzamide

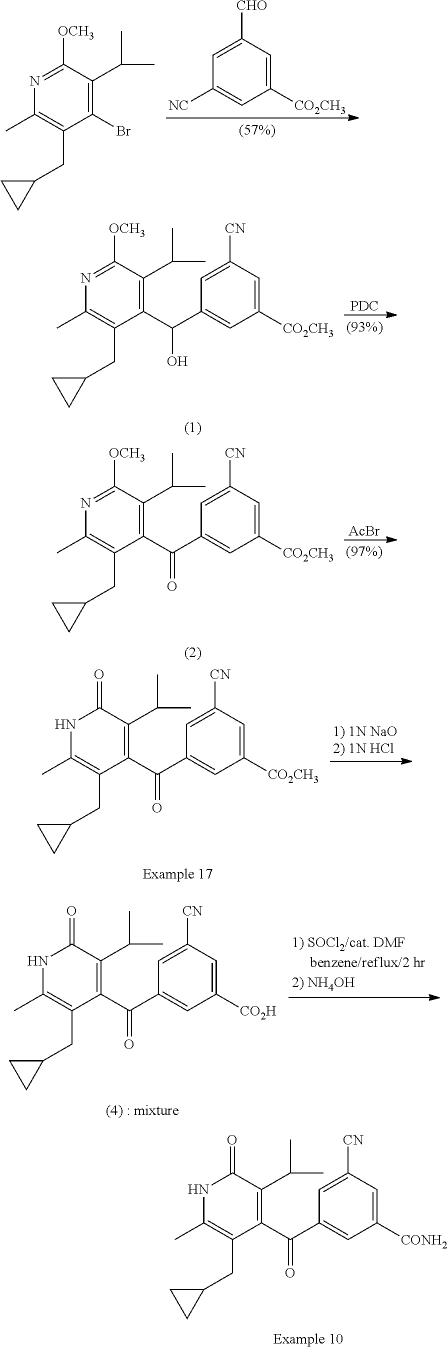

### 3-Cyano-5-[(3-cyclopropylmethyl-5-isopropyl-6-methoxy-2-methyl-pyridin-4-yl)-hydroxy-methyl]-benzoic acid methyl ester (Compound 1)

To a stirred solution of 4-bromo-3-cyclopropylmethyl-5-isopropyl-6-methoxy-2-methyl pyridine (640 mg, 2.14 mmol) in THF (10 ml) cooled in a dry ice-acetone bath (−78° C.), was added n-butyllithium(1.6M in hexane, 1.2 ml, 1.92 mmol). After stirring for 1 hr., 3-cyano-5-formylbenzoic acid methyl ester (400 mg, 2.11 mmol) in THF (4 ml) was added. The mixture was stirred at below −75° C. for 1 hr. and then stirred at room temperature for 10 min. Saturated aq. NH₄Cl solution (10 ml) was added and the product was extracted with ethyl acetate, dried (MgSO₄), filtered, and evaporated in vacuo. The residue was purified by silica gel column chromatography(eluent, ethyl acetate:hexane (1:9)) to afford 447 mg(57%) of Compound 1 as a white foam.

¹H-NMR (200 MHz, CDCl₃) δ 0.17-0.20 (2H, m), 0.46-0.51 (2H, m), 0.63 (3H, d, J=6.8 Hz), 0.70-0.84 (1H, m), 1.20 (3H, d, J=6.8 Hz), 2.50 (1H, d, J=4.4 Hz), 2.54 (3H, s), 2.67 (2H, d), 6.0 Hz), 2.94 (1H, m), 3.91 (3H, s), 3.92 (3H, s), 6.27 (1H, d, J=4.4 Hz), 7.76 (1H, s), 8.18 (2H, s).

m/z (EI) 408 (M⁺)

### 3-Cyano-5-(3-cyclopropylmethyl-5-isopropyl-6-methoxy-2-methyl-pyridin-4-carbonyl)-benzoic acid methyl ester (Compound 2)

To a stirred solution of compound 1 (537 mg, 1.31 mmol) in DMF (7 ml), was added pyridinium dichromate (742 mg, 1.97 mmol). The mixture was stirred at room temperature for overnight. The mixture was evaporated in vacuo and the residue was purified by silica gel column chromatography(eluent, ethyl acetate:hexane (1:9)) to afford 500 mg(93%) of compound 2 as a white foam.

¹H-NMR (200 MHz, CDCl₃) δ −0.17-0.18 (2H, m), 0.20-0.37 (2H, m), 0.62-0.78 (1H, m), 1.08 (3H, d, J=7.0 Hz), 1.19 (3H, d, J=7.0 Hz), 2.13 (1H, dd, J=15.4 Hz, 6.0 Hz), 2.26-2.47 (2H, m), 2.52 (3H, s), 3.98 (3H, s), 3.99 (3H, s), 8.20 (1H, s), 8.51 (1H, s), 8.65 (1H, s).

m/z (EI) 406 (M⁺)

### Example 17

### 3-Cyano-5-(5-cyclopropylmethyl-3-isopropyl-6-methyl-2-oxo-1,2-dihydro-pyridine-4-carbonyl)-benzoic acid methyl ester

A solution of compound 2 (490 mg, 1.2 mmol) in acetyl bromide (6 ml) was stirred in an oil bath (90-100° C.) for 2 hr. Reaction mixture was cooled to rt and co-evaporated with acetonitrile twice, methanol, and acetonitrile. The residue was purified by silica gel column chromatography(eluent, 2% methanol in dichloromethane) to give 460 mg (97%) of Example 17 as a pale yellow foam.

¹H-NMR (200 MHz, CDCl₃) δ −0.19-0.07 (2H, m), 0.22-0.40 (2H, m), 0.58-0.64 (1H, m), 1.19 (3H, d, J=6.6 Hz), 1.30 (3H, d, J=6.6 Hz), 2.00-2.19 (2H, m), 2.31-2.38 (1H, m), 2.42 (3H, s), 3.99 (3H, s), 8.53 (1H, s), 8.55 (1H, s), 8.70 (1H, s), 13.54 (1H, s).

m/z (EI) 392 (M⁺)

### 3-Cyano-5-(5-cyclopropylmethyl-3-isopropyl-6-methyl-2-oxo-1,2-dihydro-pyridine-4-carbonyl)-benzoic acid (Compound 4)

To a stirred solution of Example 17 (470 mg, 1.19 mmol) in methanol (15 ml), was added 1N NaOH solution (1.4 ml, 1.43 mmol). The mixture was stirred for 1 hr. at 40-50° C. (oil bath). The mixture was then cooled in an ice bath, acidified with 1N HCl solution (2 ml), and concentrated. The residue was purified by silica gel column chromatography(eluent, ethyl acetate:hexane (1:9)) to afford 247 mg of impure compound 4 as a pale yellow solid. The mixture was used directly to the next reaction.

### Example 10

### 3-Cyano-5-(5-cyclopropylmethyl-3-isopropyl-6-methyl-2-oxo-1,2-dihydro-pyridine-4-carbonyl)-benzamide

To a stirred solution of Impure Compound 4 (210 mg) in benzene (10 ml), were added thionyl chloride (1 ml) and DMF (3 drops). The mixture was then refluxed for 2 hr. After cooling to room temperature, the mixture was concentrated in vacuo and the residue was dissolved in THF (10 ml) and poured into ammonium hydroxide solution (20 ml) cooled in an ice bath. After stirring for 30 min., the mixture was concentrated under reduced pressure and the residue was purified by silica gel column chromatography(eluent, ethyl acetate:hexane (9:1)) to give 40 mg of Example 10 as a pale yellow syrup. Recrystallization from chloroform-ether-hexane resulted a pale yellow crystal.

m.p. 190° C. (dec.)

¹H-NMR (200 MHz, CDCl₃) δ −0.19-0.05 (2H, m), 0.22-0.41 (2H, m), 0.57-0.63 (1H, m), 1.16 (3H, d, J=6.8 Hz), 1.27 (3H, d, J=6.8 Hz), 2.02-2.18 (2H, m), 2.21-2.39 (4H, m), 6.69 (2H, br. s), 8.22 (1H, s), 8.43 (1H, s), 8.58 (1H, s), 12.89 (1H, s).

m/z (EI) 377 (M⁺)

### Preparation of 3-Cyano-5-formylbenzoic acid methyl ester

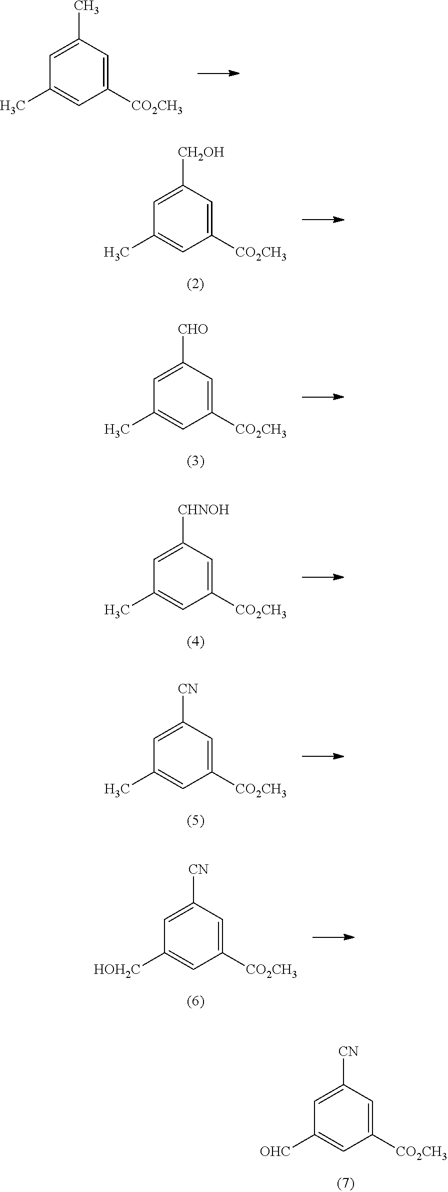

### 3-Hydroxymethyl-5-methylbenzoic acid methyl ester(2)

3,5-Dimethylbenzoic acid methyl ester (26.18 g, 0.159M) was refluxed with N-bromo succinimide (28.4 g, 0.159M) and benzoyl peroxide (1.93 g, 8.0 mmol) in CCl₄ (300 ml) under a light of 500 W tungsten lamp. After 3 hr., the mixture was cooled to room temperature, filtered, and concentrated. The residue was stirred with sodium acetate(26 g, 0.318M) in DMF (160 ml) at 70-80° C. (oil bath) for 3 hr. After cooling to room temperature, the mixture was diluted with ether, washed with water three times, dried with MgSO₄, filtered, and evaporated in vacuo. The residue was stirred with ammonium hydroxide (20 ml) in methanol (80 ml) at room temperature. After overnight, the mixture was evaporated in vacuo and the residue was purified by silica gel column chromatography(eluent, ethyl acetate:hexane (1:4)) to afford 14.4 g (50% overall) of compound 2 as a colorless oil.

¹H-NMR (200 MHz, CDCl₃) δ 1.94 (1H, t, J=6.0 Hz), 2.39 (3H, s), 3.90 (3H, s), 4.70 (2H, d, J=6.0 Hz), 7.38 (1H, s), 7.77 (1H, s), 7.82 (1H, s).

### 3-Formyl-5-methylbenzoic acid methyl ester (3)

To a stirred solution of 3-hydroxymethyl-5-methylbenzoic acid methyl ester (14.4 g, 80 mmol) in dichloromethane (160 ml), were added pyridinium chlorochromate (25.86 g, 120 mmol) and celite(30 g). After stirring for 2 hr. at room temperature, the mixture was diluted with ether and filtered through a plug of silica gel. The plug was washed with ether. The combined filtrate was evaporated in vacuo and the residue was purified by silica gel column chromatography(eluent, ether:hexane (1:9)) to give 12 g (84%) of the title compound as a white solid.

m.p. 71-73° C.

¹H-NMR (200 MHz, CDCl₃) δ 2.49 (3H, s), 3.96 (3H, s), 7.89 (1H, s), 8.12 (1H, s), 8.32 (1H, s), 10.04 (1H, s).

### 3-(Hydroxyamino-methyl)-5-methylbenzoic acid methyl ester (4)

To a stirred solution of hydroxylamine hydrochloride (2.317 g, 33.48 mmol) and triethyl amine (6 ml, 43 mmol) in methanol (60 ml), was added 3-formyl-5-methylbenzoic acid methyl ester (5.66 g, 31.76 mmol). The mixture was then heated to reflux for 2 hr. After cooling to room temperature, the mixture was concentrated under reduced pressure. The residue was dissolved in ethyl acetate, washed with water, dried with MgSO₄, filtered, and evaporated in vacuo. The residue was purified by silica gel column chromatography(eluent, ethyl acetate:hexane (1:9)) to afford 4.87 g (81%) of the title compound as a white solid.

m.p. 99-100° C.

¹H-NMR (200 MHz, CDCl₃) δ 2.41 (3H, s), 3.92 (3H, s), 7.62 (1H, s), 7.80 (1H, br. s), 7.88 (1H, s), 8.02 (1H, s), 8.15 (1H, s).

### 3-Cyano-5-methylbenzoic acid methyl ester (5)

A mixture of 3-(hydroxyamino-methyl)-5-methylbenzoic acid methyl ester (6.57 g, 34 mmol), triphenylphosphine (35.68 g, 136 mmol), and carbon tetrachloride (6.5 ml, 68 mmol) in acetonitrile (70 ml) was stirred for 1 hr. at room temperature. More carbon tetrachloride (15 ml) was then added and the mixture was stirred for 1 hr. The mixture was diluted with ether, washed with water, dried with MgSO₄, filtered, and evaporated in vacuo. The residue was purified by silica gel column chromatography(eluent, ethyl acetate:hexane (1:4)) to afford 5.7 g (95%) of the title compound as a white solid.

m.p. 68-69° C.

¹H-NMR (200 MHz, CDCl₃) δ 2.45 (3H, s), 3.95 (3H, s), 7.64 (1H, s), 8.07 (1H, s), 8.12 (1H, s)

m/z (EI) 175 (M⁺)

### 3-Cyano-5-hydroxymethylbenzoic acid methyl ester (6)

3-Cyano-5-methylbenzoic acid methyl ester (1.75 g, 10 mmol) was refluxed with N-bromo succinimide (1.78 g, 10 mmol) and benzoyl peroxide (242 mg, 1 mmol) in CCl₄ (20 ml) under a light of 500 W tungsten lamp. After 3 hr., the mixture was cooled to room temperature, filtered, and concentrated. The residue was stirred with sodium acetate (607 mg, 7.4 mmol) in DMF (10 ml) for overnight at room temperature. The mixture was diluted with ether, washed with water three times, dried with MgSO₄, filtered, and evaporated in vacuo. The residue was stirred with ammonium hydroxide (1.5 ml) in methanol (10 ml) at room temperature. After 1 hr., the mixture was evaporated in vacuo and the residue was purified by silica gel column chromatography(eluent, ethyl acetate:hexane (from 1:4 to 1:1)) to afford 790 mg(41% overall) of the title compound as a colorless oil.

¹H-NMR (200 MHz, CDCl₃) δ 2.23 (1H, t, J=5.8 Hz), 3.96 (3H, s), 4.80 (2H, d, J=5.8 Hz), 7.87 (1H, s), 8.22 (1H, s), 8.24 (1H, s).

m/z (EI) 191 (M⁺)

### 3-Cyano-5-formylbenzoic acid methyl ester (7)

To a stirred solution of 3-cyano-5-hydroxymethylbenzoic acid methyl ester (770 mg, 4 mmol) in dichloromethane (20 ml), were added pyridinium chlorochromate (1.3 g, 6 mmol) and celite (1.3 g). After stirring for 2 hr. at room temperature, the mixture was diluted with ether and filtered through a plug of silica gel. The plug was washed with ether. The combined filtrate was evaporated in vacuo and the residue was purified by silica gel column chromatography(eluent, ethyl acetate:hexane (1:4)) to afford 670 mg(88%) of the title compound as a white solid.

m.p. 144-145° C.

¹H-NMR (200 MHz, CDCl₃) δ 4.01 (3H, s), 8.35 (1H, s), 8.55 (1H, s), 8.74 (1H, s), 10.10 (1H, s).

### Example 16

### 3-(3-(5-(cyclopropylmethyl)-3-isopropyl-6-methyl-2-oxo-1,2-dihydropyridine-4-carbonyl)-5-methylphenyl)prop-2-ynyl acetate

### Example 11

### 5-(cyclopropylmethyl)-4-(3-(3-hydroxyprop-1-ynyl)-5-methylbenzoyl)-3-isopropyl-6-methylpyridin-2 (1H)-one

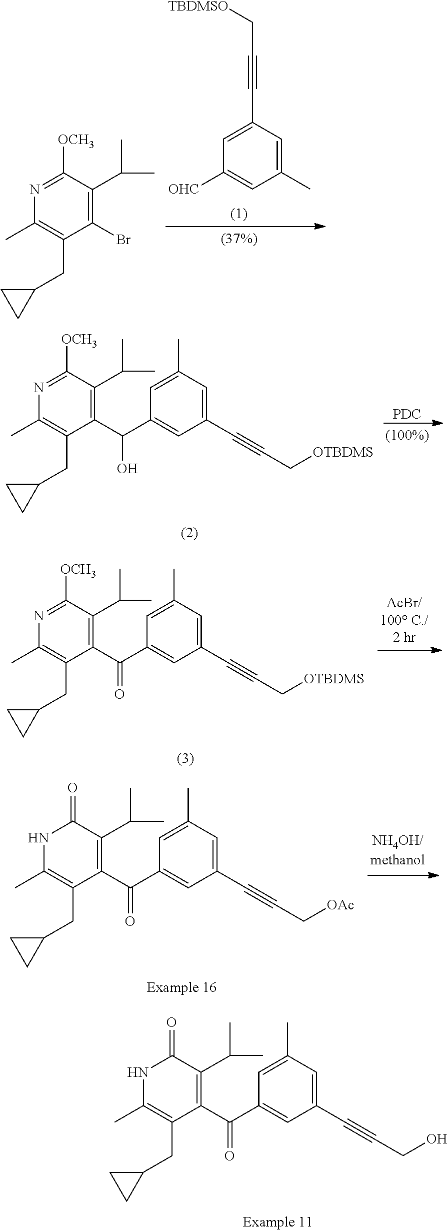

### Compound 2

To a stirred solution of 4-bromo-3-cyclopropylmethyl-5-isopropyl-6-methoxy-2-methylpyridine (298 mg, 1 mmol) in THF (8 ml) cooled in a dry ice-acetone bath (−78° C.), was added n-butyllithium(1.6M in hexane, 0.6 ml, 0.96 mmol). After stirring for 1 hr., 3-[3-(tert-butyl-dimethyl-silanyloxy)-prop-1-ynyl]-5-methyl-benzaldehyde (317 mg, 1.1 mmol) in THF (2 ml) was added. The mixture was stirred at below −75° C. for 1 hr. and then stirred at room temperature for 10 min. Saturated aq. NH₄Cl solution (10 ml) was added and the product was extracted with ethyl acetate, dried (MgSO₄), filtered, and evaporated in vacuo. The residue was purified by silica gel column chromatography(eluent, 5% ether in hexane) to afford 190 mg(37%) of compound 2 as a pale yellow syrup.

¹H-NMR (200 MHz, CDCl₃) δ 0.13-0.22 (8H, m), 0.41-0.50 (2H, m), 0.73 (3H, d, J=6.8 Hz), 0.79-0.92 (10H, m), 1.21 (3H, d, J=6.8 Hz), 2.28-2.35 (4H, m), 2.52 (3H, s), 2.62-2.67 (1H, m), 3.05-3.12 (1H, m), 3.91 (3H, s), 4.49 (2H, s), 6.21 (1H, d, J=4.8 Hz), 7.08 (2H, s), 7.10 (1H, s).

### Compound 3

To a stirred solution of compound 2 (190 mg, 0.374 mmol) in DMF (2 ml), was added pyridinium dichromate (211 mg, 0.561 mmol). The mixture was stirred at room temperature for 18 hr. The mixture was evaporated in vacuo and the residue was purified by silica gel column chromatography(eluent, 5% ether in hexane) to afford 190 mg(100%) of compound 3 as a colorless syrup.

¹H-NMR (200 MHz, CDCl₃) δ −0.13-0.12 (2H, m), 0.13 (6H, s), 0.14-0.34 (2H, m), 0.73-0.82 (1H, m), 0.91 (9H, s), 1.08 (3H, d, J=7.0 Hz), 1.19 (3H, d, J=7.0 Hz), 2.11-2.36 (5H, m), 2.43-2.53 (4H, m), 3.97 (3H, s), 4.50 (2H, s), 7.44 (1H, s), 7.55 (1H, s), 7.63 (1H, s).

m/z (EI) 505 (M⁺)

### Example 16

### 3-(3-(5-(cyclopropylmethyl)-3-isopropyl-6-methyl-2-oxo-1,2-dihydropyridine-4-carbonyl)-5-methylphenyl)prop-2-ynyl acetate

A solution of compound 3 (190 mg, 0.375 mmol) in acetyl bromide (4 ml) was stirred in an oil bath (100-110° C.) for 2 hr. Reaction mixture was cooled to rt and co-evaporated with acetonitrile twice, methanol, and acetonitrile. The residue was purified by silica gel column chromatography(eluent, ethyl acetate:hexane (1:1)) to give 130 mg(82%) of Example 16 as a white solid. The product was recrystallized from chloroform-ether to afford a pale yellow crystal.

m.p. 207=209° C.

¹H-NMR (200 MHz, CDCl₃) δ −0.18-0.05 (2H, m), 0.21-0.34 (2H, m), 0.60-0.66 (1H, m), 1.21 (3H, d, J=6.8 Hz), 1.29 (3H, d, J=6.8 Hz), 2.05-2.18 (5H, m), 2.25-2.50 (7H, m), 4.87 (2H, s), 7.48 (1H, s), 7.68 (2H, s), 13.45 (1H, br, s).

m/z (EI) 419 (M⁺)

### Example 11

### 5-(cyclopropylmethyl)-4-(3-(3-hydroxyprop-1-ynyl)-5-methylbenzoyl)-3-isopropyl-6-methylpyridin-2 (1H)-one

Example 16 (120 mg, 0.286 mmol) was stirred with ammonium hydroxide (2.5 ml) in methanol (25 ml) at room temperature for 4 hr. The mixture was then concentrated under reduced pressure and the residue was purified by silica gel column chromatography(eluent, ethyl acetate:hexane (1:1)) to give 80 mg(74%) of Example 11 as a white solid. The product was recrystallized from dichloromethane-ether-hexane to afford a white crystal.

m.p. 219-222° C.

¹H-NMR (200 MHz, CDCl₃) δ −0.14-0.05 (2H, m), 0.24-0.37 (2H, m), 0.62-0.68 (1H, m), 1.20 (3H, d, J=6.6 Hz), 1.30 (3H, d, J=6.6 Hz), 2.02-2.11 (2H, m), 2.39-2.50 (7H, m), 4.49 (2H, m), 7.48 (1H, s), 7.65 (1H, s), 7.73 (1H, s), 13.0 (1H, br.$).

m/z (EI) 377 (M⁺)

### Preparation of 3-[3-(tert-Butyl-dimethyl-silanyloxy)-prop-1-ynyl]-5-methyl-benzaldehyde (1)

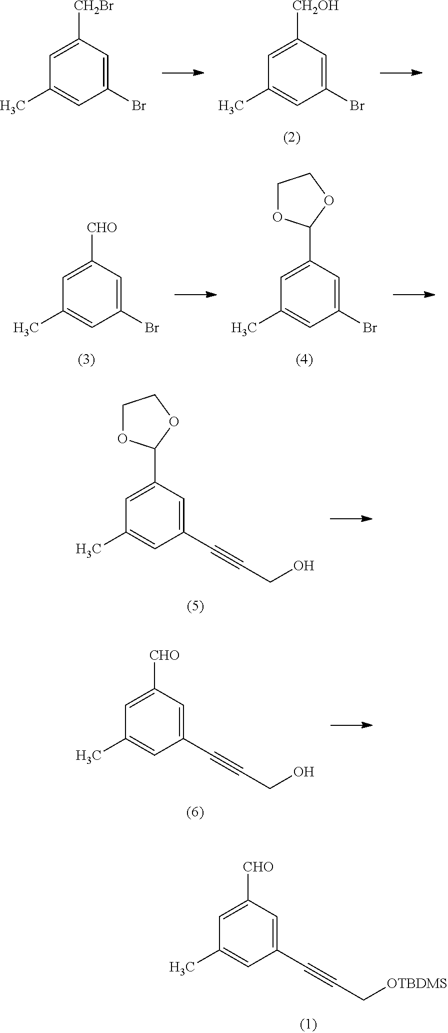

### 3-Bromo-5-methylbenzyl alcohol (2)

3-Bromo-5-methylbenzyl bromide(86 g, 0.3258M) was stirred with sodium acetate (46.5 g, 0.567M) in DMF (300 ml) for overnight at room temperature. The mixture was diluted with ether (500 ml), washed with water three times, dried with MgSO₄, filtered, and concentrated in vacuo to give a pale yellow oil. The oil was stirred with ammonium hydroxide (50 ml) in methanol (250 ml) for overnight at room temperature. The mixture was then concentrated in vacuo and the residue was purified by silica gel column chromatography(eluent, ethyl acetate:hexane (from 1:9 to 1:4)) to afford 42 g (55%) of 3-bromo-5-methylbenzyl alcohol as a colorless oil.

¹H-NMR (200 MHz, CDCl₃) δ 1.80 (1H, t, J=5.6 Hz), 2.32 (3H, s), 4.61 (2H, d, J=5.6 Hz), 7.08 (1H, s), 7.24 (1H, s), 7.31 (1H, s).

### 3-Bromo-5-methylbenzaldehyde (3)

To a stirred solution of 3-bromo-5-methylbenzyl alcohol (20.1 g, 100 mmol) in dichloromethane (200 ml), were added pyridinium chlorochromate (32 g, 150 mmol) and celite(32 g). After stirring for 2 hr. at room temperature, the mixture was diluted with ether and filtered through a plug of silica gel. The plug was washed with ether. The combined filtrate was evaporated in vacuo and the residue was purified by silica gel column chromatography(eluent, ethyl acetate:hexane (from 1:9 to 1:4)) to afford 18.3 g (92%) of 3-bromo-5-methylbenzaldehyde as a white solid.

¹H-NMR (200 MHz, CDCl₃) δ 2.44 (3H, s), 7.60 (1H, s), 7.61 (1H, s), 7.81 (1H, s), 9.94 (1H, s).

### 2-(3-Bromo-5-methyl-phenyl)-[1,3]dioxolane (4)

3-Bromo-5-methylbenzaldehyde (17 g, 85.42 mmol), ethylene glycol (9.6 ml, 170 mmol), p-toluenesulfonic acid monohydrate (1.6 g, 8.54 mmol), and toluene (200 ml) were placed in a 500 ml round bottom flask equipped with a Dean-Stark trap and a reflux condenser. The mixture was then heated to reflux for 4 hr. After cooling to room temperature, the mixture was diluted with ether, washed with sat. NaHCO₃ solution, dried with MgSO₄, filtered, and concentrated in vacuo. The residue was purified by silica gel column chromatography(eluent, ethyl acetate:hexane (1:9)) to afford 17 g (82%) of the title compound as a colorless oil.

¹H-NMR (200 MHz, CDCl₃) δ 2.36 (3H, s), 4.00-4.17 (4H, m), 5.76 (1H, s), 7.22 (1H, s), 7.34 (1H, s), 7.45 (1H, s).

### 3-(3-[1,3]Dioxolan-2-yl-5-methyl-phenyl)-prop-2-yn-1-ol (5)

A 100 ml round bottom flask fitted with a reflux condenser was charged with 2-(3-Bromo-5-methyl-phenyl)-[1,3]dioxolane (2.43 g, 10 mmol), propargyl alcohol (1.16 ml, 20 mmol), pyrrolidine (1.24 ml, 15 mmol), Pd(Ph₃P)₄ (232 mg, 0.2 mmol), and CuI (76 mg, 0.4 mmol) in distilled water (50 ml). The mixture was stirred in an oil bath (70° C.) for 1 hr. After cooling to room temperature, the mixture was diluted with ether. The organic layer was separated, dried with MgSO₄, filtered, and concentrated in vacuo. The residue was purified by silica gel column chromatography(eluent, ethyl acetate:hexane (1:4)) to afford 1.25 g (57%) of the title compound as a pale brown oil.

¹H-NMR (200 MHz, CDCl₃) δ 1.83 (1H, t, J=6.2 Hz), 2.33 (3H, s), 3.97-4.15 (4H, m), 4.46 (2H, d, J=6.2 Hz), 5.74 (1H, s), 7.25 (2H, s), 7.36 (1H, s).

m/z (EI) 218 (M⁺)

### 3-(3-Hydroxy-prop-1-ynyl)-5-methyl-benzaldehyde (6)

To a solution of 3-(3-[1,3]Dioxolan-2-yl-5-methyl-phenyl)-prop-2-yn-1-ol (1 g, 4.58 mmol) in acetone (10 ml), were added pyridinium p-toluenesulfonate (58 mg, 0.229 mmol) and 5 drops of water. The mixture was then refluxed for 4 hr. After cooling to room temperature, the mixture was concentrated in vacuo and the residue was purified by silica gel column chromatography(eluent, 2% ether in hexane) to afford 638 mg(80%) of the title compound as a colorless oil.

¹H-NMR (200 MHz, CDCl₃) δ 1.81 (1H, t, J=6.2 Hz), 2.39 (3H, s), 4.49 (2H, d, J=6.2 Hz), 7.48 (1H, s), 7.62 (1H, s), 7.71 (1H, s), 9.93 (1H, s).

### 3-[3-(tert-Butyl-dimethyl-silanyloxy)-prop-1-ynyl]-5-methyl-benzaldehyde (1)

To a stirred solution of 3-(3-Hydroxy-prop-1-ynyl)-5-methyl-benzaldehyde (470 mg, 2.698 mmol) in DMF (10 ml) cooled in an ice bath, were added imidazole (551 mg, 8 mmol) and tert-butyldimethylsilyl chloride (488 mg, 3.237 mmol). After 2 hr., the mixture was diluted with ether, washed with water, dried with MgSO₄, filtered, and concentrated in vacuo. The residue was purified by silica gel column chromatography(eluent, 2% ether in hexane) to afford 650 mg(83%) of the title compound as a pale brown oil.

¹H-NMR (200 MHz, CDCl₃) δ 0.17 (6H, s), 0.94 (9H, s), 2.41 (3H, s), 4.54 (2H, s), 7.49 (1H, s), 7.63 (1H, s), 7.72 (1H, s), 9.95 (1H, s).

### Example 12

### 3-(5-Cyclopropylmethyl-3-isopropyl-6-methyl-2-oxo-1,2-dihydro-pyridine-4-carbonyl)-5-(3-hydroxy-prop-1-ynyl)-benzonitrile

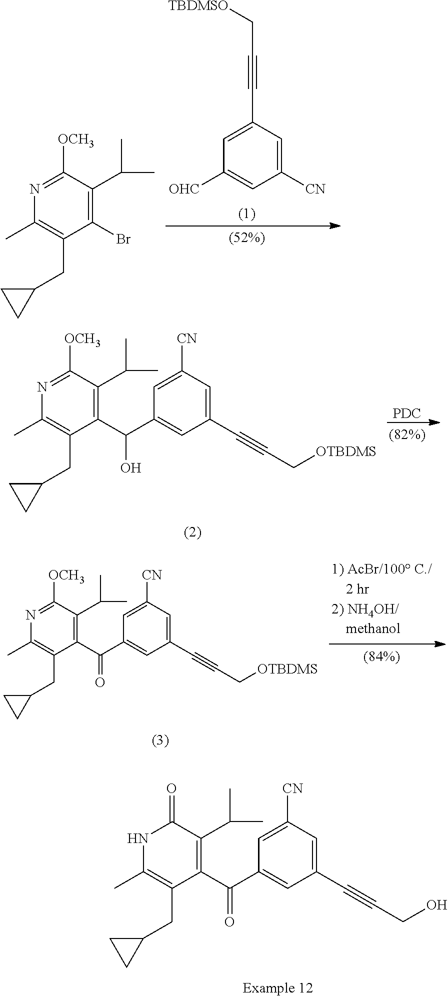

### 3-[3-(tert-Butyl-dimethyl-silanyloxy)-prop-1-ynyl]-5-[(3-cyclopropylmethyl-5-isopropyl-6-methoxy-2-methyl-pyridin-4-yl)hydroxyl-methyl]-benzonitrile (Compound 2)

To a stirred solution of 4-bromo-3-cyclopropylmethyl-5-isopropyl-6-methoxy-2-methyl-pyridine (548 mg, 1.83 mmol) in THF (10 ml) cooled in a dry ice-acetone bath (−78° C.), was added n-butyllithium(1.6M in hexane, 1.2 ml, 1.92 mmol). After stirring for 1 hr., 3-[3-(tert-butyl-dimethyl-silanyloxy)-prop-1-ynyl]-5-formyl-benzonitrile (689 mg, 2.3 mmol) in THF (5 ml) was added. The mixture was stirred at below −75° C. for 1 hr. and then stirred at room temperature for 10 min. Saturated aq. NH₄Cl solution (10 ml) was added and the product was extracted with ethyl acetate, dried (MgSO₄), filtered, and evaporated in vacuo. The residue was purified by silica gel column chromatography(eluent, ethyl acetate:hexane (from 1:19 to 1:9)) to afford 500 mg(52%) of the title compound as a colorless syrup.

¹H-NMR (200 MHz, CDCl₃) δ 0.13 (6H, s), 0.14-0.23 (2H, m), 0.40-0.47 (2H, m), 0.67 (3H, d, J=7.0 Hz), 0.72-0.0.88 (1H, m), 0.91 (9H, s), 1.20 (3H, d, J=7.0 Hz), 2.42 (1H, d, J=4.6 Hz), 2.53 (3H, s), 2.65 (2H, d, J=6.2 Hz), 2.89-3.02 (1H, m), 3.91 (3H, s), 4.50 (2H, s), 6.20 (1H, d, J=4.6 Hz), 7.47 (1H, s), 7.54 (1H, s), 7.57 (1H, s).

m/z (EI) 518 (M⁺)

### 3-[3-(tert-Butyl-dimethyl-silanyloxy)-prop-1-ynyl]-5-(3-cyclopropylmethyl-5-isopropyl-6-methoxy-2-methyl-pyridin-4-carbonyl)-benzonitrile (Compound 3)

To a stirred solution of 3-[3-(tert-butyl-dimethyl-silanyloxy)-prop-1-ynyl]-5-[(3-cyclopropylmethyl-5-isopropyl-6-methoxy-2-methyl-pyridin-4-yl)hydroxyl-methyl]-benzonitrile (500 mg, 0.96 mmol) in DMF (5 ml), was added pyridinium dichromate (544 mg, 1.44 mmol). The mixture was stirred at room temperature for overnight. The mixture was evaporated in vacuo and the residue was purified by silica gel column chromatography(eluent, ethyl acetate:hexane (1:14)) to afford 408 mg(82%) of the title compound as a colorless syrup.

¹H-NMR (200 MHz, CDCl₃) δ −0.18-0.10 (2H, m), 0.13 (6H, s), 0.23-0.37 (2H, m), 0.66-0.89 (1H, m), 0.91 (9H, s), 1.07 (3H, d, J=7.0 Hz), 1.19 (3H, d, J=7.0 Hz), 2.10 (1H, dd, J=15 hz, 5.4 Hz), 2.26-2.47 (2H, m), 2.51 (3H, s), 3.97 (3H, s), 4.52 (2H, s), 7.84 (1H, s), 7.97 (1H, s), 7.99 (1H, s).

m/z (EI) 516 (M⁺)

### Example 12

### 3-(5-Cyclopropylmethyl-3-isopropyl-6-methyl-2-oxo-1,2-dihydro-pyridine-4-carbonyl)-5-(3-hydroxy-prop-1-ynyl)-benzonitrile

A solution of 3-[3-(tert-butyl-dimethyl-silanyloxy)-prop-1-ynyl]-5-(3-cyclopropylmethyl-5-isopropyl-6-methoxy-2-methyl-pyridin-4-carbonyl)-benzonitrile (190 mg, 0.375 mmol) in acetyl bromide (4 ml) was stirred in an oil bath (100-110° C.) for 2 hr. Reaction mixture was cooled to rt and co-evaporated with acetonitrile twice, methanol, and acetonitrile. The residue was stirred with ammonium hydroxide (5 ml) in methanol (40 ml) at room temperature for 4 hr. The mixture was then concentrated under reduced pressure and the residue was purified by silica gel column chromatography(eluent, ethyl acetate:hexane (4:1)) to give 257 mg(84%) of the title compound as a yellow solid. The product was recrystallized from chloroform-ether-hexane to afford a yellow crystal.

m.p. 251-252° C.

¹H-NMR (200 MHz, DMSO-d₆) δ −0.14-0.15 (2H, m), 0.20-0.28 (2H, m), 0.50-0.68 (1H, m), 1.05 (3H, d, J=6.6 Hz), 1.14 (3H, d, J=6.6 Hz), 1.86 (1H, dd, J=14.8 Hz, 5.0 Hz), 2.03-2.14 (2H, m), 2.23 (3H, s), 4.31 (2H, d, J=5.8 Hz), 5.42 (1H, t, J=5.8 Hz), 7.93 (1H, s), 8.26 (2H, s), 11.69 (1H, s).

m/z (EI) 388 (M⁺)

### Preparation of 3-[3-(tert-Butyl-dimethyl-silanyloxy)-prop-1-ynyl]-5-formyl-benzonitrile (1)

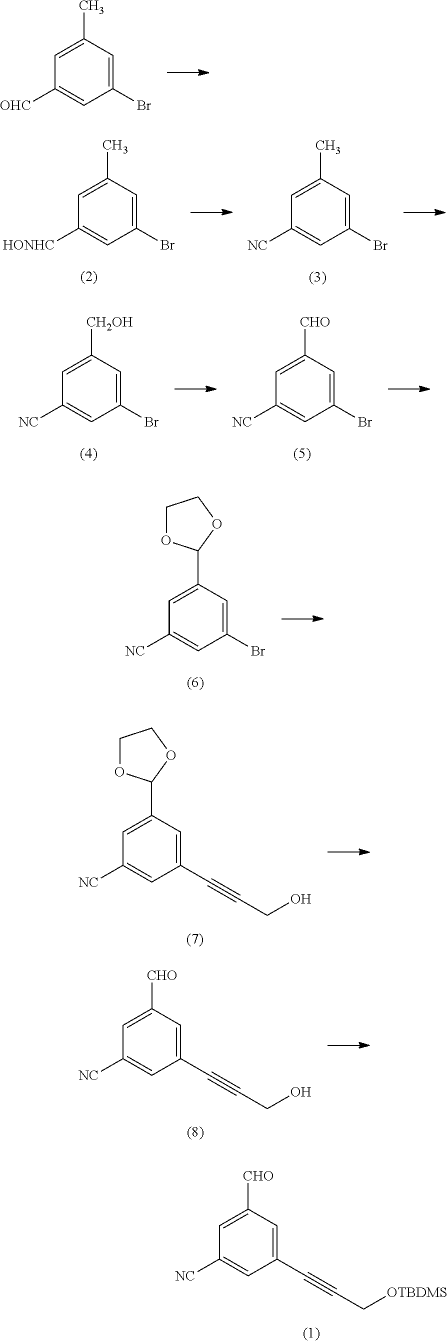

### 3-Bromo-5-methylbenzaldehyde oxime (2)

To a stirred solution of hydroxylamine hydrochloride (7.0 g, 101 mmol) and triethyl amine (16.6 ml, 119 mmol) in ethanol (150 ml), was added 3-bromo-5-methylbenzaldehyde (18.3 g, 92 mmol). The mixture was then heated to reflux for 2 hr. After cooling to room temperature, the mixture was concentrated under reduced pressure. The residue was dissolved in dichloromethane, washed with water, dried with MgSO₄, filtered, and evaporated in vacuo. The residue was purified by silica gel column chromatography(eluent, ethyl acetate:hexane (1:14)) to afford 18 g (91%) of the title compound as a white solid.

m.p. 128-129° C.

¹H-NMR (200 MHz, CDCl₃) δ 2.34 (3H, s), 7.30 (1H, s), 7.35 (1H, s), 7.53 (1H, s), 7.66 (1H, s), 8.04 (br. s).

### 3-Bromo-5-methylbenzonitrile (3)

A mixture of 3-bromo-5-methylbenzaldehyde oxime (18 g, 84 mmol), triphenylphosphine (88 g, 336 mmol), and carbon tetrachloride (16.2 ml, 168 mmol) in acetonitrile (160 ml) was stirred for 1 hr. in an ice-water bath. More carbon tetrachloride (30 ml) was then added and the mixture was stirred for 1 hr. The mixture was diluted with ether, washed with water, dried with MgSO₄, filtered, and evaporated in vacuo. The residue was purified by silica gel column chromatography(eluent, ethyl acetate:hexane (1:9)) to afford 15 g (93%) of the title compound as a white solid.

m.p. 85-86° C.

¹H-NMR (200 MHz, CDCl₃) δ 2.38 (3H, s), 7.39 (1H, s), 7.56 (1H, s), 7.58 (1H, s).

m/z (EI) 212 (M⁺)

### 3-Bromo-5-hydroxymethylbenzonitrile (4)

3-Bromo-5-methylbenzonitrile (15 g, 76.5 mmol) was refluxed with N-bromo succinimide (13.6 g, 76.5 mmol) and benzoyl peroxide (925 mg, 3.8 mmol) in CCl₄ (100 ml) under a light of 500 W tungsten lamp. After 4 hr., the mixture was cooled to room temperature, filtered, and concentrated. The residue was stirred with sodium acetate (9.41 g, 115 mmol) in DMF (100 ml) for overnight at room temperature. The mixture was diluted with ether, washed with water three times, dried with MgSO₄, filtered, and evaporated in vacuo. The residue was stirred with ammonium hydroxide (30 ml) in methanol (100 ml) at room temperature. After 3 hr., the mixture was evaporated in vacuo and the residue was purified by silica gel column chromatography(eluent, ethyl acetate:hexane (1:4)) to afford 7.1 g (44% overall) of the title compound as a white solid.

m.p. 108-109° C.

¹H-NMR (200 MHz, CDCl₃) δ 2.03 (1H, t, J=5.6 Hz), 4.73 (2H, d, J=5.6 Hz), 7.60 (1H, s), 7.70 (1H, s), 7.76 (1H, s).

m/z (EI) 212 (M⁺)

### 3-Bromo-5-formylbenzonitrile (5)

To a stirred solution of 3-bromo-5-hydroxymethylbenzonitrile (7.1 g, 33.48 mmol) in dichloromethane (100 ml), were added pyridinium chlorochromate (10.8 g, 50 mmol) and celite(10 g). After stirring for 2 hr. at room temperature, the mixture was diluted with ether and filtered through a plug of silica gel. The plug was washed with ether. The combined filtrate was evaporated in vacuo and the residue was purified by silica gel column chromatography(eluent, ethyl acetate:hexane (1:4)) to afford 6.4 g (91%) of the title compound as a white solid.

m.p. 134-135° C.

¹H-NMR (200 MHz, CDCl₃) δ 8.05 (1H, s), 8.11 (1H, s), 8.24 (1H, s), 9.99 (1H, s).

m/z (EI) 210 (M⁺)

### 3-Bromo-5-[1,3]dioxolan-2-yl-benzonitrile (6)

3-Bromo-5-formylbenzonitrile (3 g, 14.28 mmol), ethylene glycol (1.61 ml, 28.56 mmol), p-toluenesulfonic acid monohydrate (136 mg, 0.71 mmol), and benzene (80 ml) were placed in a 250 ml round bottom flask equipped with a Dean-Stark trap and a reflux condenser. The mixture was then heated to reflux for 4 hr. After cooling to room temperature, the mixture was diluted with ether, washed with sat. NaHCO₃ solution, dried with MgSO₄, filtered, and concentrated in vacuo to afford 3.6 g (100%) of the title compound as a pale yellow oil. The product was used directly to the next reaction.

¹H-NMR (200 MHz, CDCl₃) δ 4.03-4.11 (4H, m), 5.79 (1H, s), 7.71 (1H, s), 7.77 (1H, s), 7.84 (1H, s).

m/z (EI) 254 (M⁺)

### 3-[1,3]dioxolan-2-yl-5-(3-hydroxyprop-1-ynyl)-benzonitrile (7)

A 100 ml round bottom flask fitted with a reflux condenser was charged with 3-bromo-5-[1,3]dioxolan-2-yl-benzonitrile (2.54 g, 10 mmol), propargyl alcohol (1.16 ml, 20 mmol), pyrrolidine (1.24 ml, 15 mmol), Pd(Ph₃P)₄ (232 mg, 0.2 mmol), and CuI(76 mg, 0.4 mmol) in distilled water (50 ml). The mixture was stirred in an oil bath (70° C.) for 1 hr. After cooling to room temperature, the mixture was diluted with ether. The organic layer was separated, dried with MgSO₄, filtered, and concentrated in vacuo. The residue was purified by silica gel column chromatography(eluent, ethyl acetate:hexane (1:3)) to afford 1.2 g (52%) of the title compound as a pale brown syrup.

¹H-NMR (200 MHz, CDCl₃) δ 1.81 (1H, t, J=6.4 Hz), 4.00-4.15 (4H, m), 4.49 (2H, d, J=6.4 Hz), 5.80 (1H, s), 7.68 (1H, s), 7.72 (1H, s), 7.75 (1H, s).

m/z (EI) 229 (M⁺)

### 3-Formyl-5-(3-hydroxy-prop-1-ynyl)-benzonitrile (8)

To a solution of 3-[1,3]dioxolan-2-yl-5-(3-hydroxyprop-1-ynyl)-benzonitrile (1.2 g, 5.23 mmol) in acetone (10 ml), were added p-toluenesulfonic acid monohydrate (470 mg, 2.47 mmol). The mixture was then refluxed for 4 hr. After cooling to room temperature, the mixture was stirred with excess NaHCO₃ for 30 min. The mixture was then concentrated in vacuo and the residue was purified by silica gel column chromatography(eluent, ethyl acetate:hexane (1:2)) to afford 600 mg(62%) of the title compound as a pale yellow syrup.

¹H-NMR (200 MHz, CDCl₃) δ 1.88 (1H, t, J=6.4 Hz), 4.53 (2H, d, J=6.4 Hz), 7.92 (1H, s), 8.09 (1H, s), 8.12 (1H, s), 10.00 (1H, s).

### 3-[3-(tert-Butyl-dimethyl-silanyloxy)-prop-1-ynyl]-5-formyl-benzonitrile (1)

To a stirred solution of 3-formyl-5-(3-hydroxy-prop-1-ynyl)-benzonitrile (600 mg, 3.24 mmol) in DMF (10 ml) cooled in an ice bath, were added imidazole (662 mg, 9.72 mmol) and tert-butyldimethylsilyl chloride (733 mg, 4.86 mmol). After 2 hr., the mixture was diluted with ether, washed with water, dried with MgSO₄, filtered, and concentrated in vacuo. The residue was purified by silica gel column chromatography(eluent, ether:hexane (1:2)) to afford 760 mg(78%) of the title compound as a white solid.

m.p. 73-74° C.

¹H-NMR (200 MHz, CDCl₃) δ 0.17 (6H, s), 0.94 (9H, s), 2.41 (3H, s), 4.55 (2H, s), 7.90 (1H, s), 8.08 (1H, s), 8.11 (1H, s), 10.01 (1H, s).

m/z (EI) 299 (M⁺)

### Example 13

### 3-(5-Cyclopropylmethyl-6-hydroxymethyl-3-isopropyl-2-oxo-1,2-dihydro-pyridine-4-carbonyl)-5-methyl-benzonitrile

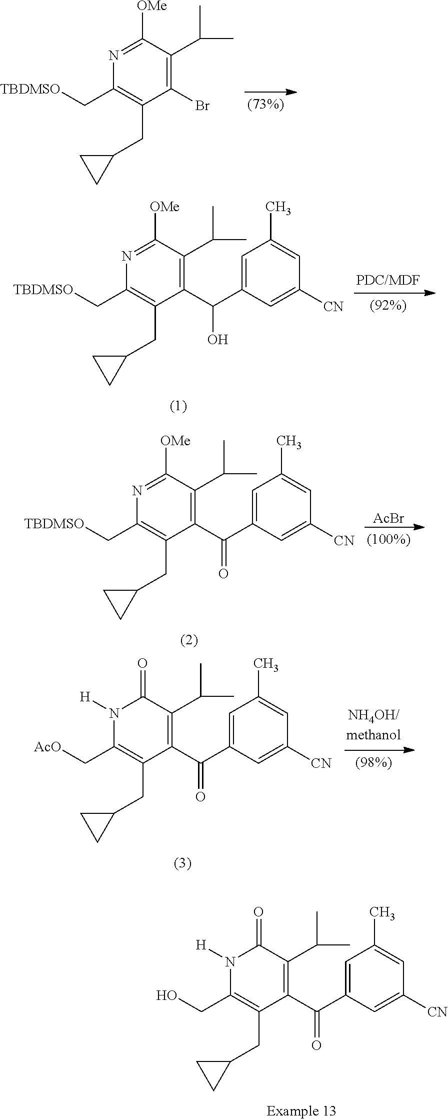

### 3-{([2-tert-Butyl-dimethyl-silanyloxymethyl]-3-cyclopropylmethyl-5-isopropyl-6-methoxy-pyridin-4-yl]-hydroxy-methyl}-5-methyl-benzonitrile (1)

To a stirred solution of 4-bromo-2-(tert-butyl-dimethyl-silanyloxymethyl)-3-cyclopropylmethyl-5-isopropyl-6-methoxy-pyridine (294 mg, 0.686 mmol) in THF (6 ml) cooled in a dry ice-acetone bath (−78° C.), was added n-butyllithium(1.6M in hexane, 0.4 ml, 0.64 mmol). After stirring for 1 hr., 3-cyano-5-methylbenzaldehyde (119 mg, 0.82 mmol) in THF (2 ml) was added. The mixture was stirred at below −75° C. for 50 min. and then stirred at room temperature for 10 min. Saturated aq. NH₄Cl solution (10 ml) was added and the product was extracted with ethyl acetate, dried with MgSO₄, filtered, and evaporated in vacuo. The residue was purified by silica gel column chromatography(eluent, ether:hexane (1:19)) to afford 260 mg(82%) of the title compound as a colorless foam.

¹H-NMR (200 MHz, CDCl₃) δ 0.11 (3H, s), 0.12 (3H, s), 0.20-0.24 (2H, m), 0.43-0.49 (2H, m), 0.65 (3H, d, J=7.0 Hz), 0.79-0.90 (1H, m), 0.92 (9H, s), 1.20 (3H, d, J=7.0 Hz), 2.33-2.35 (4H, m), 2.76-2.84 (2H, m), 2.99-3.06 (1H, m), 3.93 (3H, s), 4.76 (1H, d, J=11.4 Hz), 4.83 (1H, d, J=11.4 Hz), 6.28 (1H, d, J=4.0 Hz), 7.26 (1H, s), 7.33 (1H, s), 7.42 (1H, s).

m/z (EI) 494 (M⁺)

### 3-[(2-tert-Butyl-dimethyl-silanyloxymethyl)-3-cyclopropylmethyl-5-isopropyl-6-methoxy-pyridin-4-carbonyl]-5-methyl-benzonitrile (2)

To a stirred solution of 3-{[2-tert-butyl-dimethyl-silanyloxymethyl]-3-cyclopropylmethyl-5-isopropyl-6-methoxy-pyridin-4-yl]-hydroxy-methyl}-5-methyl-benzonitrile (240 mg, 0.485 mmol) in DMF (4 ml), was added pyridinium dichromate (274 mg, 0.727 mmol). The mixture was stirred at room temperature for 18 hr. The mixture was evaporated in vacuo and the residue was purified by silica gel column chromatography(eluent, ether:hexane (1:19)) to afford 220 mg (92%) of the title compound as a colorless syrup.

¹H-NMR (200 MHz, CDCl₃) δ −0.11-0.08 (2H, m), 0.13 (3H, s), 0.14 (3H, s), 0.20-0.33 (2H, m), 0.71-0.88 (1H, m), 0.92 (9H, s), 1.07 (3H, d, J=7.0 Hz), 1.20 (3H, d, J=7.0 Hz), 2.20 (1H, dd, J=14.6 Hz, 5.4 Hz), 2.40-2.54 (4H, m), 4.00 (3H, s), 4.73 (1H, d, J=11.8 Hz), 4.87 (1H, d, J=11.8 Hz), 7.67 (1H, s), 7.84 (2H, s).

m/z (EI) 492 (M⁺)

### Acetic acid 4-(3-cyano-5-methyl-benzoyl)-3-cyclopropylmethyl-5-isopropyl-6-oxo-1,6-dihydro-pyridin-2-ylmethyl ester (3)

A mixture of 3-[(2-tert-Butyl-dimethyl-silanyloxymethyl)-3-cyclopropylmethyl-5-isopropyl-6-methoxy-pyridin-4-carbonyl]-5-methyl-benzonitrile (200 mg, 0.406 mmol) and acetyl bromide (4 ml) was refluxed for 2 hr. Reaction mixture was cooled to rt and co-evaporated with acetonitrile three times, methanol, and acetonitrile. The residue was purified by silica gel column chromatography(eluent, ethyl acetate:hexane (from 1:2 to 1:1)) to give 170 mg(100%) of the title compound as a pale yellow syrup.

¹H-NMR (200 MHz, CDCl₃) δ −0.17-0.09 (2H, m), 0.25-0.42 (2H, m), 0.56-0.63 (1H, m), 1.17 (3H, d, J=7.0 Hz), 1.28 (3H, d, J=7.0 Hz), 2.10-2.17 (5H, m), 2.31-2.48 (4H, m), 5.00 (1H, d, J=13.2 Hz), 5.10 (1H, d, J=13.2 Hz), 7.71 (1H, s), 7.92 (1H, s), 7.95 (1H, s), 11.85 (1H, s).

m/z (EI) 406 (M⁺)

### Example 13

### 3-(5-Cyclopropylmethyl-6-hydroxymethyl-3-isopropyl-2-oxo-1,2-dihydro-pyridine-4-carbonyl)-5-methyl-benzonitrile

Acetic acid 4-(3-cyano-5-methyl-benzoyl)-3-cyclopropylmethyl-5-isopropyl-6-oxo-1,6-dihydro-pyridin-2-ylmethyl ester (170 mg, 0.418 mmol) was stirred with NH₄OH(0.5 ml) in methanol (5 ml) at room temperature. After 2 hr., the mixture was evaporated in vacuo and the residue was purified by silica gel column chromatography(eluent, ethyl acetate) to give 150 mg(98%) of the title compound as a white foam. The product was recrystallized from ether-hexane to give a yellow solid.

m.p. 169-170° C.

¹H-NMR (200 MHz, CDCl₃) δ −0.15-0.10 (2H, m), 0.27-0.43 (2H, m), 0.56-0.65 (1H, m), 1.18 (3H, d, J=7.0 Hz), 1.28 (3H, d, J=7.0 Hz), 2.02-2.19 (2H, m), 2.39-2.53 (4H, m), 4.75 (2H, s), 7.71 (1H, s), 7.90 (1H, s), 7.94 (1H, s).

m/z (EI) 364 (M⁺)

### Example 18

### Acetic acid 3-cyclopropylmethyl-4-(3,5-dicyano-benzoyl)-5-isopropyl-6-oxo-1,6-dihydro-pyridin-2-ylmethyl ester

### Example 14

### 5-(5-Cyclopropylmethyl-6-hydroxymethyl-3-isopropyl-2-oxo-1,2-dihydro-pyridine-4-carbonyl)-isophthalonitrile

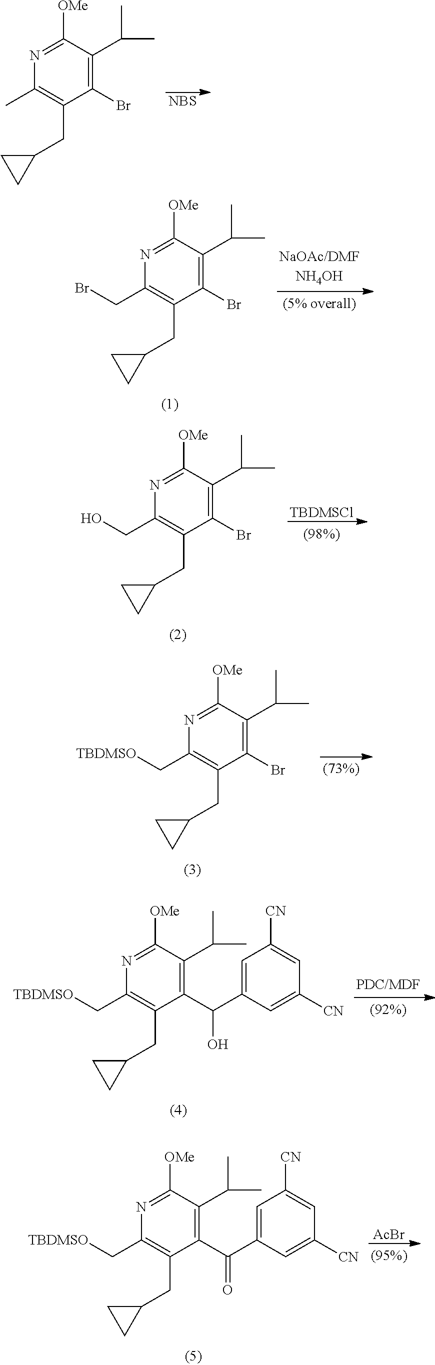

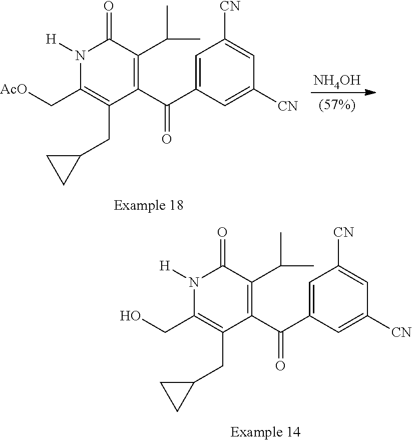

### (4-Bromo-3-cyclopropylmethyl-5-isopropyl-6-methoxy-pyridin-2-yl)-methanol (2)

4-Bromo-3-cyclopropylmethyl-5-isopropyl-6-methoxy-2-methyl-pyridine (10 g, 33.55 mmol) was refluxed with N-bromo succinimide (5.97 g, 33.55 mmol) and benzoyl peroxide (812 mg, 3.34 mmol) in CCl₄ (60 ml) under a light of 500 W tungsten lamp. After 2 hr., the mixture was cooled to room temperature, filtered, and concentrated. The residue was purified by silica gel column chromatography(eluent, 1% ether in hexane) to afford 2.18 g of 4-(bromo-2-bromomethyl-3-cyclopropylmethyl-5-isopropyl-6-methoxy-pyridine as a mixture of starting material. The mixture was stirred with sodium acetate (1.9 g, 23 mmol) in DMF (10 ml) for overnight at room temperature. The mixture was diluted with ether, washed with water three times, dried with MgSO₄, filtered, and evaporated in vacuo. The residue was stirred with ammonium hydroxide (10 ml) in methanol (40 ml) at room temperature. After 3 hr., the mixture was evaporated in vacuo and the residue was purified by silica gel column chromatography(eluent, ether:hexane (1:9)) to afford 600 mg(5% overall) of the title compound as a colorless syrup.

¹H-NMR (300 MHz, CDCl₃) δ 0.20-0.30 (2H, m), 0.45-0.49 (2H, m), 0.94-0.99 (1H, m), 1.30 (6H, d, J=7.0 Hz), 2.65 (2H, d, J=6.3 Hz), 3.63-3.72 (1H, m), 3.98 (3H, s), 4.37 (1H, t, J=4.5 Hz), 4.69 (2H, d, J=4.5 Hz).

m/z (EI) 314 (M⁺)

### 4-Bromo-2-(tert-butyl-dimethyl-silanyloxymethyl)-3-cyclopropylmethyl-5-isopropyl-6-methoxy-pyridine (3)

To a stirred solution of (4-bromo-3-cyclopropylmethyl-5-isopropyl-6-methoxy-pyridin-2-yl)-methanol (590 mg, 1.87 mmol) in DMF (6 ml) cooled in an ice bath, were added imidazole (383 mg, 5.63 mmol) and tert-butyldimethylsilyl chloride (425 mg, 2.81 mmol). After 1 hr., the mixture was diluted with ether, washed with water, dried with MgSO₄, filtered, and concentrated in vacuo. The residue was purified by silica gel column chromatography(eluent, ether:hexane (1:9)) to afford 790 mg(98%) of the title compound as a colorless oil.

¹H-NMR (300 MHz, CDCl₃) δ 0.01 (6H, s), 0.19-0.20 (2H, m), 0.31-0.35 (2H, m), 0.81 (9H, s), 0.90-1.00 (1H, m), 1.18 (6H, d, J=7.0 Hz), 2.77 (2H, d, J=6.3 Hz), 3.52-3.57 (1H, m), 3.82 (3H, s), 4.62 (3H, s).

### 5-{[2-tert-Butyl-dimethyl-silanyloxymethyl]-3-cyclopropylmethyl-5-isopropyl-6-methoxy-pyridin-4-yl]-hydroxy-methyl}-isophthalonitrile (4)

To a stirred solution of 4-bromo-2-(tert-butyl-dimethyl-silanyloxymethyl)-3-cyclopropylmethyl-5-isopropyl-6-methoxy-pyridine (428 mg, 1 mmol) in THF (8 ml) cooled in a dry ice-acetone bath (−78° C.), was added n-butyllithium(1.6M in hexane, 0.6 ml, 0.96 mmol). After stirring for 1 hr., 3,5-dicyanobenzaldehyde (187 mg, 1.2 mmol) in THF (2 ml) was added. The mixture was stirred at below −75° C. for 50 min. and then stirred at room temperature for 10 min. Saturated aq. NH₄Cl solution (10 ml) was added and the product was extracted with ethyl acetate, dried with MgSO₄, filtered, and evaporated in vacuo. The residue was purified by silica gel column chromatography(eluent, ethyl acetate:hexane (1:9)) to afford 370 mg(73%) of the title compound as a white foam.

¹H-NMR (200 MHz, CDCl₃) δ 0.13 (6H, s), 0.14-0.38 (2H, m), 0.40-0.56 (2H, m), 0.62 (3H, d, J=7.0 Hz), 0.76-0.91 (1H, m), 0.92 (9H, s), 1.20 (3H, d, J=7.0 Hz), 2.50-2.60 (1H, br. s), 2.80 (2H, d, J=6.0 Hz), 2.89 (1H, m), 3.93 (3H, s), 4.80 (2H, s), 6.29 (1H, d, J=4.0 Hz), 7.80 (3H, s).

m/z (EI) 505 (M⁺)

### 5-[(2-tert-Butyl-dimethyl-silanyloxymethyl)-3-cyclopropylmethyl-5-isopropyl-6-methoxy-pyridine-4-carbonyl]-isophthalonitrile (5)

To a stirred solution of 5-{[2-tert-butyl-dimethyl-silanyloxymethyl]-3-cyclopropylmethyl-5-isopropyl-6-methoxy-pyridin-4-yl]-hydroxy-methyl}-isophthalonitrile (370 mg, 0.73 mmol) in DMF (5 ml), was added pyridinium dichromate (413 mg, 1.1 mmol). The mixture was stirred at room temperature for 16 hr. The mixture was evaporated in vacuo and the residue was purified by silica gel column chromatography(eluent, ethyl acetate:hexane (from 1:19 to 1:15)) to afford 340 mg(92%) of the title compound as a white foam.

¹H-NMR (200 MHz, CDCl₃) δ −0.15-0.09 (2H, m), 0.13 (6H, s), 0.19-0.37 (2H, m), 0.67-0.73 (1H, m), 0.92 (9H, s), 1.08 (3H, d, J=7.0 Hz), 1.20 (3H, d, J=7.0 Hz), 2.19 (1H, dd, J=15.2 Hz, 5.6 Hz), 2.35-2.55 (2H, m), 4.01 (3H, s), 4.73 (1H, d, J=12.0 Hz), 4.86 (1H, d, J=12.0 Hz), 8.12 (1H, s), 8.25 (2H, s).

m/z (EI) 503 (M⁺)

### Example 18

### Acetic acid 3-cyclopropylmethyl-4-(3,5-dicyano-benzoyl)-5-isopropyl-6-oxo-1,6-dihydro-pyridin-2-ylmethyl ester

A mixture of 5-[2-tert-butyl-dimethyl-silanyloxymethyl]-3-cyclopropylmethyl-5-isopropyl-6-methoxy-pyridine-4-carbonyl]-isophthalonitrile (330 mg, 0.65 mmol) and acetyl bromide (5 ml) was refluxed for 2 hr. Reaction mixture was cooled to room temperature and co-evaporated with acetonitrile three times, methanol, and acetonitrile. The residue was purified by silica gel column chromatography(eluent, ethyl acetate:hexane (1:2˜1:1)) to give 260 mg(95%) of the title compound as a white foam.

¹H-NMR (200 MHz, CDCl₃) δ −0.17-0.11 (3H, m), 0.23-0.47 (2H, m), 0.54-0.58 (1H, m), 1.18 (3H, d, J=6.8 Hz), 1.28 (3H, d, J=6.8 Hz), 2.01-2.23 (5H, m), 2.29-2.39 (1H, m), 5.01 (1H, d, J=13.0 Hz), 5.11 (1H, d, J=13.0 Hz), 8.17 (1H, s), 8.34 (2H, s), 12.26 (1H, s).

m/z (EI) 417 (M⁺)

### Example 14

### 5-(5-Cyclopropylmethyl-6-hydroxymethyl-3-isopropyl-2-oxo-1,2-dihydro-pyridine-4-carbonyl)-isophthalonitrile

Acetic acid 3-cyclopropylmethyl-4-(3,5-dicyano-benzoyl)-5-isopropyl-6-oxo-1,6-dihydro-pyridin-2-ylmethyl ester (250 mg, 0.6 mmol) was stirred with NH₄OH(2 ml) in methanol (10 ml) at room temperature. After 2 hr., the mixture was evaporated in vacuo and the residue was purified by silica gel column chromatography(eluent, ethyl acetate) to give 130 mg(57%) of the title compound as a pale yellow syrup. The product was recrystallized from chloroform-ether-hexane to give a white solid.

m.p. 157-158° C.

¹H-NMR (200 MHz, CDCl₃) δ −0.15-0.09 (2H, m), 0.29-0.44 (2H, m), 0.57-0.59 (1H, m), 1.19 (3H, d, J=6.8 Hz), 1.29 (3H, d, J=6.8 Hz), 1.99-2.11 (2H, m), 2.36-2.43 (1H, m), 4.77 (2H, s), 8.18 (1H, s), 8.35 (2H, s).

m/z (EI) 375 (M⁺)

### Example 15

### 3-(6-Cyanomethyl-5-cyclopropylmethyl-3-isopropyl-2-oxo-1,2-dihydro-pyridine-4-carbonyl)-5-methyl-benzonitrile

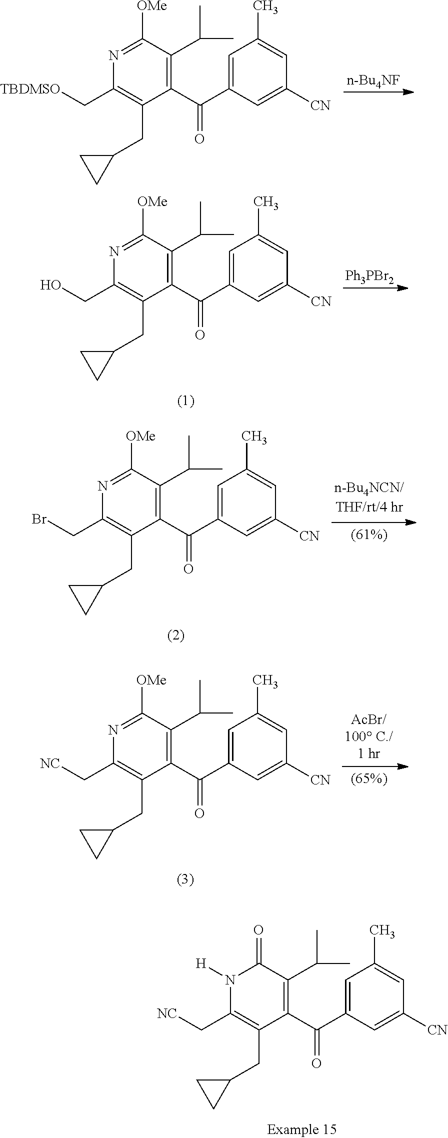

### 3-(3-Cyclopropylmethyl-2-hydroxymethyl-5-isopropyl-6-methoxy-pyridine-4-carbonyl)-5-methyl-benzonitrile (1)

To a stirred solution of 3-[(2-tert-butyl-dimethyl-silanyloxymethyl)-3-cyclopropylmethyl-5-isopropyl-6-methoxy-pyridin-4-carbonyl]-5-methyl-benzonitrile (240 mg, 0.487 mmol) in THF (2 ml) at room temperature, was added tetrabutylammonium fluoride(1M in THF, 0.8 ml, 0.8 mmol). After 3 hr., the mixture was evaporated in vacuo and the residue was purified by silica gel column chromatography(eluent, ether:hexane (from 1:1 to 2:1)) to afford 190 mg(100%) of the title compound as a white syrup.

¹H-NMR (300 MHz, CDCl₃) δ −0.14-0.07 (1H, m), 0.00-0.08 (1H, m), 0.23-0.39 (2H, m), 0.60-0.65 (1H, m), 1.10 (3H, d, J=6.9 Hz), 1.24 (3H, d, J=6.9 Hz), 2.11 (1H, dd, J=14.9 Hz, 5.6 Hz), 2.20 (1H, dd, J=14.9 Hz, 5.6 Hz), 2.48-2.55 (4H, m), 4.06 (3H, s), 4.36 (1H, t, J=4.5 Hz), 4.76 (2H, d, J=4.5 Hz), 7.69 (1H, s), 7.82 (1H, s), 7.86 (1H, s).

m/z (EI) 378 (M⁺)

### 3-(2-Bromomethyl-3-cyclopropylmethyl-5-isopropyl-6-methoxy-pyridine-4-carbonyl)-5-methyl-benzonitrile (2)

To a stirred solution of 3-(3-cyclopropylmethyl-2-hydroxymethyl-5-isopropyl-6-methoxy-pyridine-4-carbonyl)-5-methyl-benzonitrile (173 mg, 0.457 mmol) in dichloromethane (5 ml) in an ice bath, was added triphenylphosphine dibromide (289 mg, 0.685 mmol). After 1.5 hr., the mixture was evaporated in vacuo and the residue was purified by silica gel column chromatography(eluent, ether:hexane (1:1)) to afford 150 mg(74%) of the title compound as a white syrup.

¹H-NMR (300 MHz, CDCl₃) δ −0.11-0.04 (1H, m), 0.04-0.11 (1H, m), 0.27-0.42 (2H, m), 0.71-0.76 (1H, m), 1.09 (3H, d, J=6.8 Hz), 1.21 (3H, d, J=6.8 Hz), 2.25 (1H, dd, J=15.0 Hz, 5.6 Hz), 2.40-2.525H, m), 4.01 (3H, s), 4.56 (1H, d, J=9.7 Hz), 4.63 (1H, d, J=9.7 Hz), 7.69 (1H, s), 7.82 (1H, s), 7.84 (1H, s).

### 3-(2-Cyanomethyl-3-cyclopropylmethyl-5-isopropyl-6-methoxy-pyridine-4-carbonyl)-5-methyl-benzonitrile (3)

To a stirred solution of 3-(2-bromomethyl-3-cyclopropylmethyl-5-isopropyl-6-methoxy-pyridine-4-carbonyl)-5-methyl-benzonitrile (150 mg, 0.340 mmol) in THF (5 ml) at room temperature, was added tetrabutylammonium cyanide (137 mg, 0.510 mmol). After 4 hr., the mixture was evaporated in vacuo and the residue was purified by silica gel column chromatography(eluent, ether:hexane (from 1:2 to 1:1)) to afford 80 mg(61%) of the title compound as a white syrup.

¹H-NMR (300 MHz, CDCl₃) δ −0.12-0.01 (1H, m), 0.01-0.04 (1H, m), 0.24-0.38 (2H, m), 0.57-0.63 (1H, m), 1.03 (3H, d, J=6.8 Hz), 1.14 (3H, d, J=6.8 Hz), 2.12 (1H, dd, J=15.2 Hz, 5.4 Hz), 2.23 (1H, dd, J=15.2 Hz, 5.4 Hz), 2.38-2.47 (4H, m), 3.87 (2H, s), 3.96 (3H, s), 7.64 (1H, s), 7.77 (2H, s).

m/z (EI) 387 (M⁺)

### Example 15

### 3-(6-Cyanomethyl-5-cyclopropylmethyl-3-isopropyl-2-oxo-1,2-dihydro-pyridine-4-carbonyl)-5-methyl-benzonitrile

A mixture of 3-(2-cyanomethyl-3-cyclopropylmethyl-5-isopropyl-6-methoxy-pyridine-4-carbonyl)-5-methyl-benzonitrile (80 mg, 0.206 mmol) and acetyl bromide (2 ml) was refluxed for 1 hr. Reaction mixture was cooled to rt and co-evaporated with acetonitrile three times, methanol, and acetonitrile. The residue was purified by silica gel column chromatography(eluent, ethyl acetate:hexane (from 1:1 to 2:1)) to give 50 mg(65%) of the title compound as a pale brown solid. The product was recrystallized from chloroform-ether-hexane to give a pale brown solid.

m.p. 168-169° C.

¹H-NMR (300 MHz, CDCl₃) δ −0.11-0.03 (1H, m), 0.05-0.13 (1H, m), 0.30-0.50 (2H, m), 0.62-0.69 (1H, m), 1.15 (3H, d, J=6.8 Hz), 1.32 (3H, d, J=6.8 Hz), 2.17-2.21 (2H, m), 2.41-2.49 (4H, m), 7.80 (1H, d, J=17.5 Hz), 3.87 (1H, d, J=17.5 Hz), 7.73 (1H, s), 7.88 (1H, s), 7.97 (1H, s), 13.75 (1H, s).

m/z (EI) 373 (M⁺)

The following prophetic compounds may be prepared according to Sheme P4 or by modifications of the routes described herein or in Scheme 1-5.

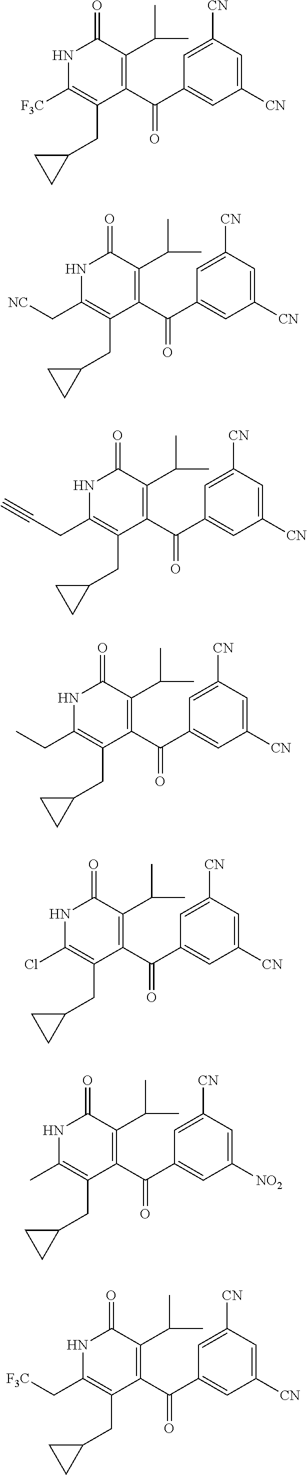

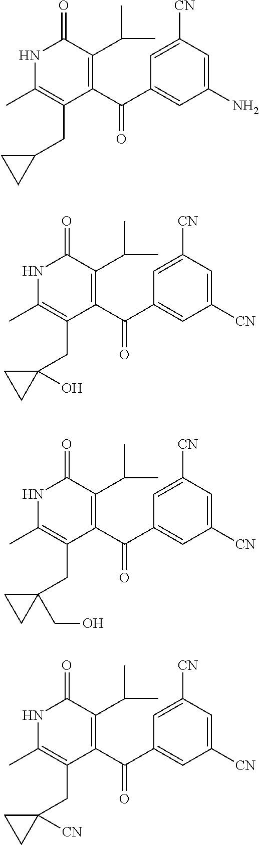

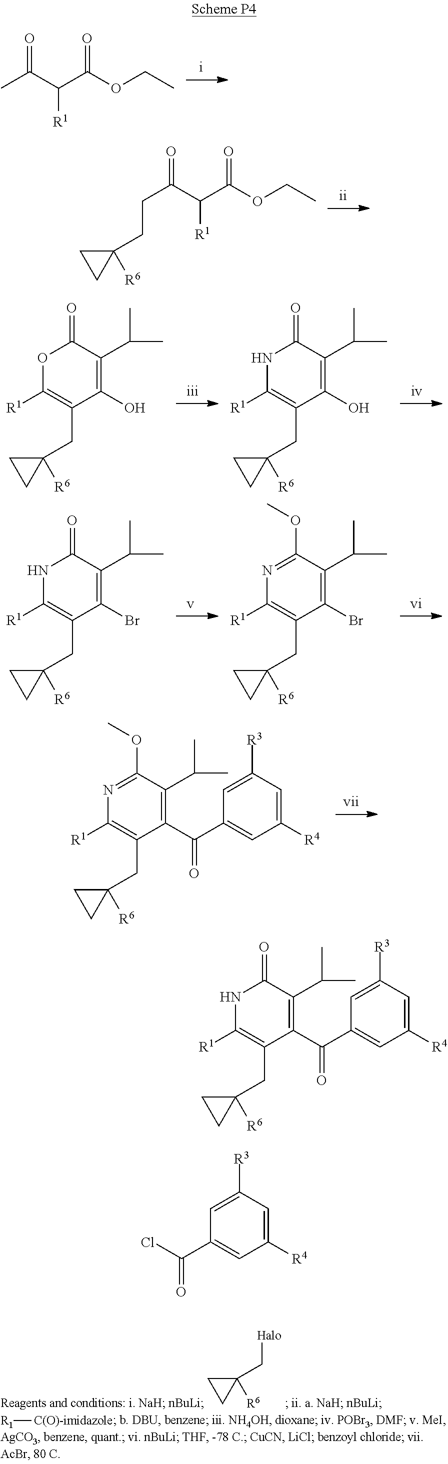

### BIOLOGICAL EXAMPLES

### Antiviral and Cytotoxicity Assays in MT2 and MT4 Cells

For the antiviral assay utilizing MT-2 cells, 50 μl of 2× test concentration of 5-fold serially diluted compound in culture medium with 10% FBS was added to each well of a 96-well plate (9 concentrations) in triplicate. MT-2 cells were infected with HIV-IIIb at a multiplicity of infection (m.o.i) of 0.01 for 3 hours. Fifty microliters of infected cell suspension in culture medium with 10% FBS (˜1.5×10⁴ cells) was then added to each well containing 50 μl of diluted compound. The plates were then incubated at 37° C. for 5 days. For the antiviral assay utilizing MT-4 cells, 20 μl of 2× test concentration of 5-fold serially diluted compound in culture medium with 10% FBS was added to each well of a 384-well plate (7 concentrations) in triplicate. MT-4 cells were next mixed with HIV-IIIb at an m.o.i. of 0.1 and 20 μl of virus/cell mixture (˜2000 cells) was immediately added to each well containing 20 μl of diluted compound. The plates were then incubated at 37° C. for 5 days. After 5 days of incubation, 100 μl of CellTiter-Glo™ Reagent (catalog #G7571, Promega Biosciences, Inc., Madison, Wis.) was added to each well containing MT-2 cells and 40 μl to each well containing MT-4 cells. Cell lysis was carried out by incubation at room temperature for 10 min and chemiluminescence was read.

For compound cytotoxicity assessment in MT-2 cells, the protocol was identical to that of the antiviral assay in MT-2 cells, except that uninfected cells and a 3-fold serial dilution of compounds were used. For cytotoxicity assessment in MT-4 cells, the protocol is identical to that of the antiviral assay in MT-4 cells, except that no virus was added. Cytotoxicity is expressed as CC₅₀. In a preferred aspect, the CC₅₀ of the compounds of the present invention is greater than 30 μM, more preferably greater than 50 μM, and most preferably greater than 100 μM.

Compounds of the present invention demonstrate an EC₅₀ of <100 nM. In certain embodiments, the compounds demonstrate an EC₅₀ of <50 μM. In certain embodiments, the compounds demonstrate an EC₅₀ of <30 μM. In certain embodiments, the compounds demonstrate an EC₅₀ of <10 μM.

Representative EC₅₀ and CC₅₀ for the compounds of the instant invention is shown in Table I wherein an EC₅₀ less than 100 nM is represented by A, 101-1000 nM is B, and greater than 1000 nM is represented by C.

TABLE 1
EC₅₀ and CC₅₀ data for Examples 1-15.

| Example | EC₅₀ | CC₅₀ |
|---|---|---|
| 1 | A | — |
| 2 | A | — |
| 3 | A | — |
| 5 | A | — |
| 6 | A | >100 |
| 7 | A | — |
| 8 | A | — |
| 9 | A | >69 |
| 10 | A | — |
| 11 | A | — |
| 12 | A | — |
| 13 | A | — |
| 14 | A | — |
| 15 | A | — |

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I: 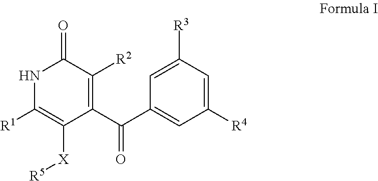
or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R² is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
X is alkylene, substituted alkylene, alkenylene, substituted alkenylene, alkynylene, or substituted alkynylene;
R⁵ is cycloalkyl, substituted cycloalkyl, aryl, substituted aryl, heterocyclyl, substituted heterocyclyl, heteroaryl, or substituted heteroaryl;
or R¹ and R⁵ and X can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)R¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

2. The compound of claim 1, wherein R¹ is alkyl, alkynyl, haloalkyl, (Q)ₘ-hydroxy, or (Q)ₘ-cyano.

3. The compound of claim 1, wherein R² is alkyl.

4. The compound of claim 1, represented by Formula II

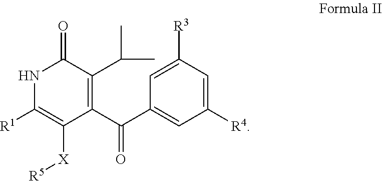

5. The compound of claim 1, wherein X is alkylene.

6. The compound of claim 1, represented by Formula III

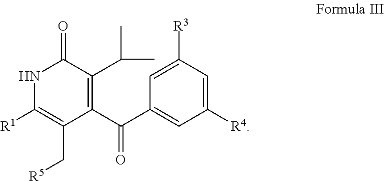

7. The compound of claim 1, wherein R³ is alkyl, haloalkyl, halogen, cyano, nitro, amino, alkylamino, or dialkylamino.

8. The compound of claim 1, wherein R⁴ is alkyl, substituted alkynyl, haloalkyl, halogen, cyano, —C(O)OR¹⁰, or —C(O)N(R¹⁰)₂.

9. The compound of claim 1, wherein R⁵ is cycloalkyl or substituted cycloalkyl.

10. The compound of claim 1, wherein R⁵ is cycloalkyl substituted with one or more (Q)ₘ-hydroxyl, —C(O)R¹⁰, (Q)ₘ-OC(O)R¹⁰ or (Q)ₘ-cyano.

11. The compound of claim 1, wherein R⁵ is cyclopropyl or substituted cyclopropyl.

12. The compound of claim 1, wherein R⁵ is cyclopropyl substituted with one or more (Q)ₘ-hydroxyl, —C(O)R¹⁰, (Q)ₘ-OC(O)R¹⁰ or (Q)ₘ-cyano.

13. The compound of claim 1, wherein R⁵ is

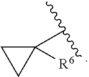

14. The compound of claim 13, wherein R⁶ is selected from (Q)ₘ-hydroxyl, —C(O)R¹⁰, (Q)ₘ-OC(O)R¹⁰ or (Q)ₘ-cyano.

15. The compound of claim 1, wherein each Q is independently CH₂ wherein m is 0 or 1.

16. The compound of claim 1, wherein each R³ and R⁴ is independently selected from methyl, chloro or cyano.

17. The compound of claim 1, wherein R¹ is methyl.

18. A compound of Formula IV 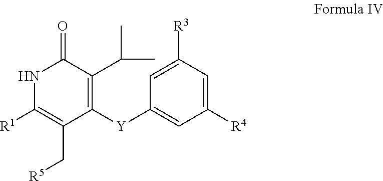
or a pharmaceutically acceptable salt or prodrug thereof, wherein
R¹ is alkyl, alkenyl, alkynyl, (Q)ₘ-hydroxy, (Q)ₘ-oxo, (Q)ₘ-alkoxy, (Q)ₘ-halogen, (Q)ₘ-haloalkyl, (Q)ₘ-amino, (Q)ₘ-alkylamino, (Q)ₘ-dialkylamino, (Q)ₘ-cyano, (Q)ₘ-nitro, (Q)ₘ-cycloalkyl, or (Q)ₘ-substituted cycloalkyl;
R³ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
R⁴ is alkyl, substituted alkyl, alkenyl, substituted alkenyl, alkynyl, substituted alkynyl, alkoxy, halogen, haloalkyl, hydroxyl, amino, alkylamino, dialkylamino, cyano, nitro, C(O)R¹⁰, CO₂R¹⁰, S(O)_{q}R¹⁰, OC(O)R¹⁰, OC(O)OR¹⁰, C(O)N(R¹⁰)₂, NR¹⁰C(O)R¹⁰, NR¹⁰C(O)OR¹⁰, (Q)ₘ-cycloalkyl, (Q)ₘ-substituted cycloalkyl, (Q)ₘ-aryl, (Q)ₘ-substituted aryl, (Q)ₘ-heterocyclyl, (Q)ₘ-substituted heterocyclyl, (Q)ₘ-heteroaryl, or (Q)ₘ-substituted heteroaryl;
Y is —C(O)—, —S(O)_{q}—, —O—, —N(R¹⁰)—, —C(R¹⁰)₂—, or —CF₂—;
R⁵ is cycloalkyl, substituted cycloalkyl, aryl, substituted aryl, heterocyclyl, substituted heterocyclyl, heteroaryl, or substituted heteroaryl;
or R¹ and R⁵ can combine with the atoms to which they are attached to form a 5- to 7-membered ring that may include one or more N, O, or S heteroatom and may further be substituted with one or more R⁶;
wherein each of substituted alkyl, substituted alkylene, substituted alkenyl, substituted alkenylene, substituted alkynyl, substituted alkynylene, substituted cycloalkyl, substituted aryl, substituted heterocyclyl, and substituted heteroaryl is substituted with one or more R⁶;
each R⁶ independently is alkyl, alkenyl, alkynyl, (Q)ₘ-alkoxy, (Q)ₘ-halogen, (Q)ₘ-haloalkyl, (Q)ₘ-hydroxyl, (Q)ₘ-oxo, (Q)ₘ-amino, (Q)ₘ-alkylamino, (Q)ₘ-dialkylamino, (Q)ₘ-cyano, (Q)ₘ-nitro, (Q)ₘ-C(O)R¹⁰, (Q)ₘ-CO₂R¹⁰, (Q)ₘ-S(O)_{q}R¹⁰, (Q)ₘ-OC(O)R¹⁰, (Q)ₘ-OC(O)OR¹⁰, (Q)ₘ-C(O)N(R¹⁰)₂, (Q)ₘ-NR¹⁰C(O)OR¹⁰, (Q)ₘ-NR¹⁰C(O)OR¹⁰, (Q)ₘ-cycloalkyl, (Q)ₘ-aryl, (Q)ₘ-heterocyclyl, or (Q)ₘ-heteroaryl;
each Q independently is alkylene, substituted alkylene, alkenylene, substituted alkenylene, alkynylene, or substituted alkynylene;
each m independently is 0-6;
each q independently is 0, 1, or 2; and
each R¹⁰ independently is hydrogen, alkyl, alkenyl, alkynyl, amino, alkylamino, dialkylamino, cycloalkyl, aryl, aralkyl, heterocyclyl, heteroaryl, or heteroaralkyl.

19. The compound of claim 18, wherein R¹ is alkyl, alkynyl, haloalkyl, (Q)ₘ-hydroxy, or (Q)ₘ-cyano.

20. The compound of claim 18, wherein R³ is alkyl, haloalkyl, halogen, cyano, nitro, amino, alkylamino, or dialkylamino.

21. The compound of claim 18, wherein R⁴ is alkyl, substituted alkynyl, haloalkyl, halogen, cyano, —C(O)OR¹⁰, or —C(O)N(R¹⁰)₂.

22. The compound of claim 18, wherein Y is —C(O)— or —S(O)_{q}—.

23. The compound of claim 18, wherein R⁵ is cycloalkyl or substituted cycloalkyl

24. The compound of claim 23, wherein R⁵ is cycloalkyl substituted with one or more (Q)ₘ-hydroxyl, —C(O)R¹⁰, (Q)ₘ-OC(O)R¹⁰ or (Q)ₘ-cyano.

25. The compound of claim 18 represented by Formula V

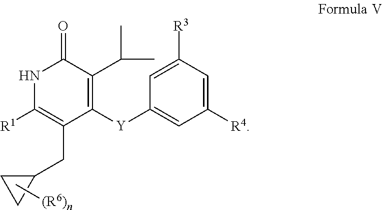

26. The compound of claim 18, wherein R⁵ is

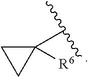

27. The compound of claim 26, wherein R⁶ is selected from (Q)ₘ-hydroxyl, —C(O)R¹⁰, (Q)ₘ-OC(O)R¹⁰ or (Q)ₘ-cyano.

28. The compound of claim 18, wherein each Q is independently CH₂ wherein m is 0 or 1.

29. The compound of claim 18, wherein each R³ and R⁴ is independently selected from methyl, chloro or cyano.

30. The compound of claim 18, wherein R¹ is methyl.

31. A compound according to claim 1 that is 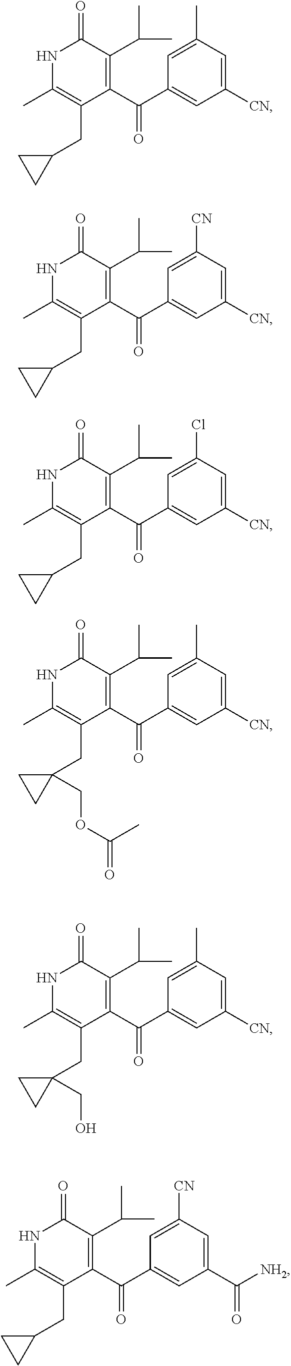 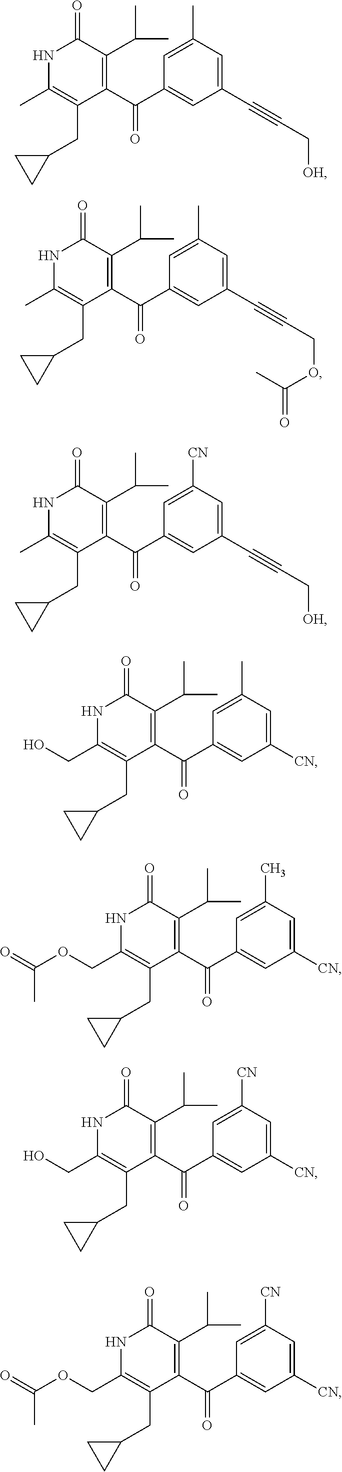 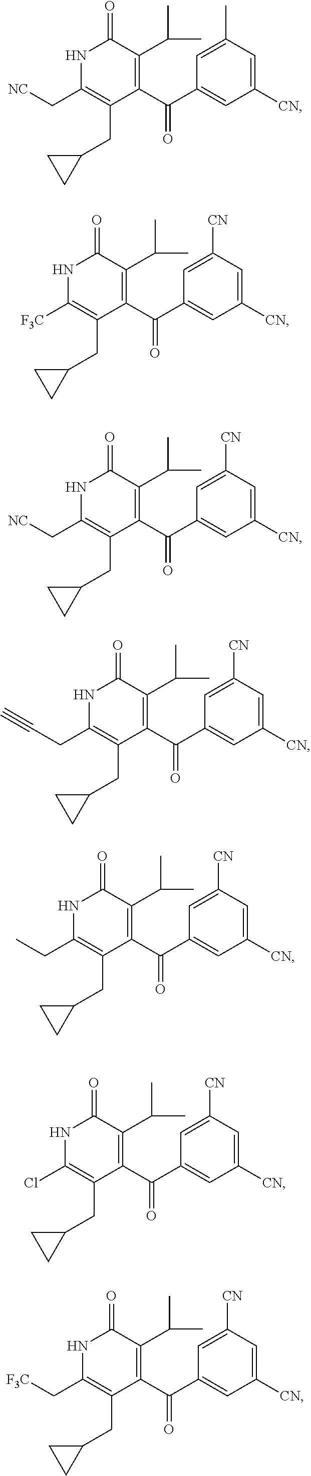 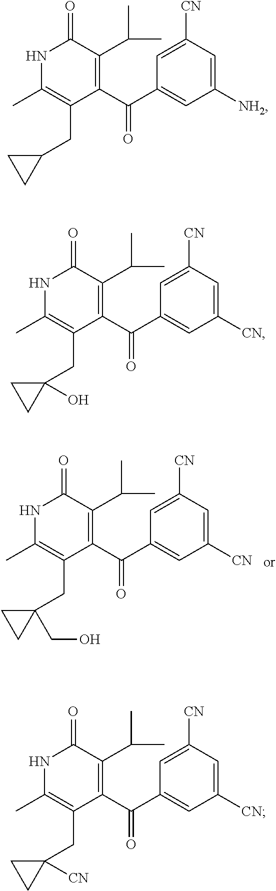
or a pharmaceutically acceptable salt thereof.

32. A compound according to claim 18 that is 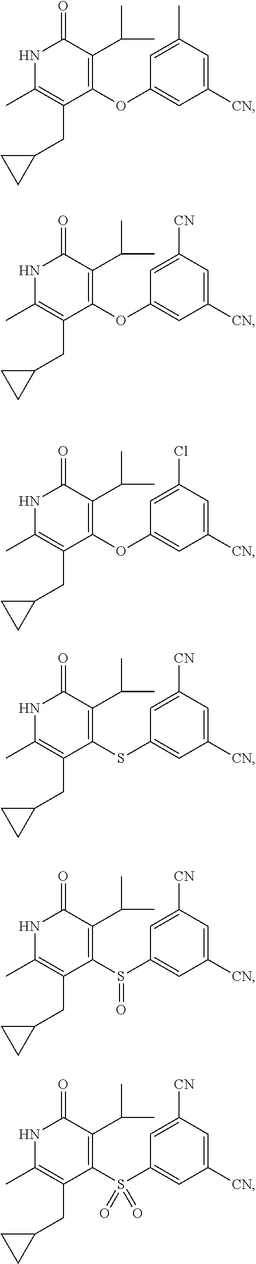 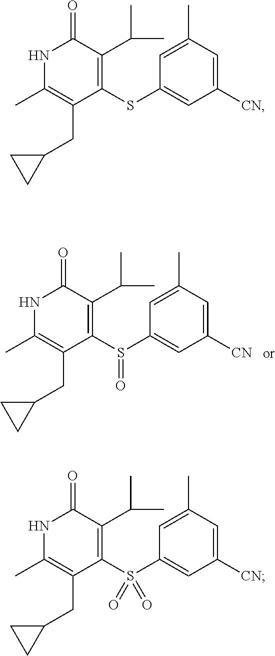
or a pharmaceutically acceptable salt thereof.

33. A pharmaceutical composition comprising a compound as claimed in claim 1 and one or more pharmaceutically acceptable carrier.

34. The pharmaceutical composition of claim 33 comprising one or more additional therapeutic agent.

35. The pharmaceutical composition of claim 34, wherein the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

36. A method for inhibiting HIV reverse transcriptase comprising the administration of a compound as claimed in claim 1.

37. A method for the treatment or prevention of HIV infection comprising the administration of a compound as claimed in claim 1.

38. A method for treating or preventing AIDS or AIDS-Related Complex comprising the administration of a compound as claimed in claim 1.

39. A method for inhibiting replication of a retrovirus comprising the administration of a compound as claimed in claim 1.

40. The method of claim 1, further comprising the administration of one or more additional therapeutic agent.

41. The method of claim 40, wherein the one or more additional therapeutic agent is an HIV protease inhibitor, HIV non-nucleoside inhibitor of reverse transcriptase, HIV nucleoside inhibitor of reverse transcriptase, HIV nucleotide inhibitor of reverse transcriptase, HIV integrase inhibitor, gp41 inhibitor, CXCR4 inhibitor, entry inhibitor, gp120 inhibitor, G6PD and NADH-oxidase inhibitor, CCR5 inhibitor, CCR8 inhibitor, RNase H inhibitor, maturation inhibitor, pharmacokinetic enhancer, or other drugs for treating HIV.

42-54. (canceled)
